(19) United States
(12) Patent Application Publication
(10) Pub. No.: US 2021/0085640 A1
(43) Pub. Date: Mar. 25, 2021

(54) ACYLATED CATECHIN POLYPHENOLS AND METHODS OF THEIR USE FOR THE TREATMENT OF CANCER

(71) Applicant: Flagship Pioneering Innovations V, Inc., Cambridge, MA (US)

(72) Inventors: Steven John TAYLOR, Winchester, MA (US); Leonard BUCKBINDER, East Greenwich, RI (US); John Robert PROUDFOOT, Newtown, CT (US); Mi-Jeong KIM, Boston, MA (US); Timothy F. BRIGGS, Waltham, MA (US); Ferdinand Edward MASSARI, Beverly, MA (US); Spencer Cory PECK, Watertown, MA (US); Koji YASUDA, Boston, MA (US); Amir MOAREFI, Wellesley Hills, MA (US); David Arthur BERRY, Waban, MA (US); John Patrick CASEY, JR., Boston, MA (US)

(21) Appl. No.: 17/110,741

(22) Filed: Dec. 3, 2020

Related U.S. Application Data
(63) Continuation of application No. PCT/US2019/035670, filed on Jun. 5, 2019.
(60) Provisional application No. 62/776,391, filed on Dec. 6, 2018.
(60) Provisional application No. 62/680,973, filed on Jun. 5, 2018.

(51) Int. Cl. A61K 31/353; A61K 31/222; A61K 31/351; A61K 31/7048; A61K 31/22; A61K 31/265; A61P 35/00

(52) U.S. Cl. CPC A61K 31/353; A61K 31/222; A61K 31/351; A61P 35/00; A61K 31/22; A61K 31/265; A61K 31/7048

(57) ABSTRACT

Disclosed herein are acylated active agents and methods of their use, e.g., for modulating a cancer marker or for treating cancer.

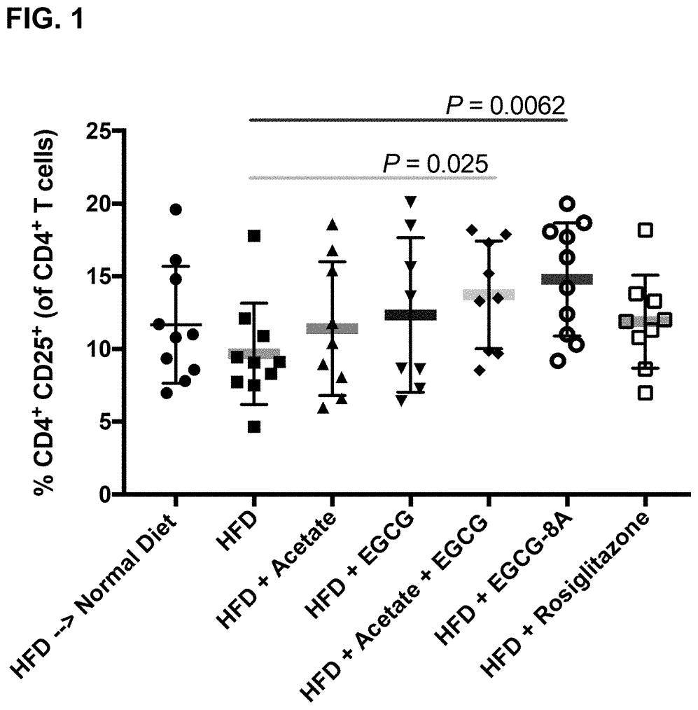

### FIELD OF THE INVENTION

The invention relates compounds and methods of their medicinal use.

### BACKGROUND

Recent developments in cancer treatment utilizing the host's immune system have primarily focused on antigen-binding proteins. Small molecule-based therapeutic approaches for recruiting the host's immune system remain underutilized. Small molecule-based therapies, however, can offer advantages in structural flexibility, manufacturing, and characterization. There is a need for new approaches to cancer treatment.

### SUMMARY OF THE INVENTION

In general, the invention provides acylated active agents and compositions containing them (e.g., as unit dosage forms), and methods for modulating a cancer marker in a subject or of treating a cancer in a subject. The acylated active agent is an acylated catechin polyphenol, acylated stilbenoid, acylated ellagic acid analogue, or acylated ketone body or pre-ketone body.

In one aspect, the invention provides a method of modulating a cancer marker in a subject in need thereof by administering to the subject an effective amount of an acylated active agent. In a related aspect, the invention provides a method of treating a cancer in a subject in need thereof by administering to the subject an effective amount of an acylated active agent. The acylated active agent is an acylated catechin polyphenol, acylated stilbenoid, acylated ellagic acid analogue, or acylated ketone body or pre-ketone body.

In some embodiments, the cancer marker is for a cancer selected from the group consisting of stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, and colorectal cancer. In certain embodiments, the cancer stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, or colorectal cancer. In some embodiments, the cancer marker is for a cancer selected from the group consisting of stomach cancer, skin cancer, prostate cancer, lung cancer, breast cancer, non-Hodgkin lymphoma, and bladder cancer. In other embodiments, the cancer is stomach cancer, skin cancer, prostate cancer, lung cancer, breast cancer, non-Hodgkin lymphoma, or bladder cancer.

In further embodiments, a CD4⁺CD25⁺ Treg cell count, cytotoxic T cell count, interferon γ (IFNγ) level, interleukin-17 (IL17) level, or intercellular adhesion molecule (ICAM) level is increased following the administration of the acylated active agent. In yet further embodiments, an NFκB level, matrix metallopeptidase 9 (MMP9) level, 8-iso-prostaglandin F_{2α} (8-iso-PGF2α) level, or CXCL13 level is reduced following the administration of the acylated active agent. In still further embodiments, a Tₕ1 cell count, IgA level, or inducible nitric oxide synthase (iNOS) level is modulated following the administration of the acylated active agent.

In particular embodiments, following oral administration to the subject, the acylated active agent is cleavable (e.g., hydrolyzable) in the gastrointestinal tract of the subject. In certain embodiments, the acylated active agent releases at least one fatty acid. In further embodiments, the acylated active agent is administered to the subject orally.

In other embodiments, the acylated active agent includes a group containing a fatty acid. In yet other embodiments, the group containing a fatty acid is a monosaccharide (e.g., arabinose, xylose, fructose, galactose, glucose, glucosinolate, ribose, tagatose, fucose, and rhamnose), sugar alcohol, or sugar acid having one or more hydroxyl groups substituted with a fatty acid acyl). In still other embodiments, the monosaccharide is L-arabinose, D-xylose, fructose, galactose, D-glucose, glucosinolate, D-ribose, D-tagatose, L-fucose, or L-rhamnose (e.g., the monosaccharide is D-xylose). In further embodiments, the group containing a fatty acid is a fatty acid acyl (e.g., a C₃₋₅ fatty acid acyl). In yet further embodiments, the fatty acid is a short chain fatty acid (e.g., a C₃₋₅ fatty acid (e.g., propionyl, butyryl, or valeryl)). In still further embodiments, the short chain fatty acid is acetyl. In particular embodiments, the short chain fatty acid is propionyl, butyryl, or valeryl (preferably, butyryl).

In yet further embodiments, the acylated active agent is an acylated catechin polyphenol. In still further embodiments, the acylated catechin polyphenol is a compound of formula (I):

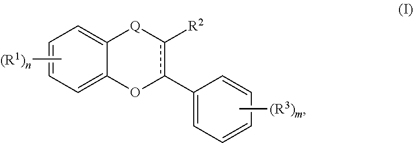

or a pharmaceutically acceptable salt thereof,

where

[symbol] is a single carbon-carbon bond or double carbon-carbon bond;

Q is —CH₂— or —C(O)—;

each R¹ and each R³ is independently H, halogen, —OR^{A}, phosphate, or sulfate;

R² is H or —OR^{A};

each R^{A} is independently H, optionally substituted alkyl, a monosaccharide, a sugar acid, a group containing a fatty acid, or benzoyl optionally substituted with 1, 2, 3, or 4 substituents independently selected from the group consisting of H, hydroxy, halogen, a group containing a fatty acid, an optionally substituted alkyl, an optionally substituted alkoxy, a monosaccharide, a sugar acid, phosphate, and sulfate;

each of n and m is independently 0, 1, 2, 3, or 4.

In particular embodiments, the compound of formula (I) includes at least one group containing a fatty acid.

In some embodiments, at least one R¹ is —OR^{A}, in which R^{A} is a group containing a fatty acid.

In certain embodiments, the acylated catechin polyphenol is a compound is of formula (I-a):

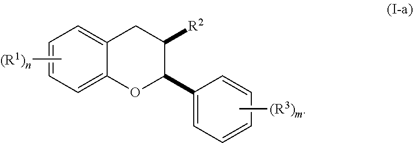

In particular embodiments, the acylated catechin polyphenol is a compound is of formula (I-b):

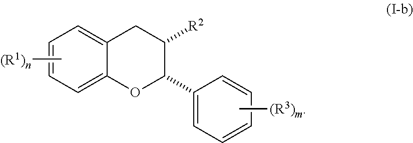

In further embodiments, the acylated catechin polyphenol is a compound is of formula (I-c):

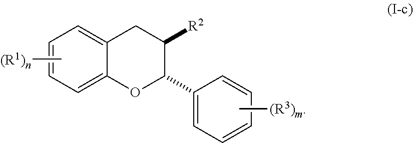

In yet further embodiments, the acylated catechin polyphenol is a compound is of formula (I-d):

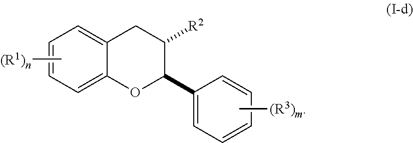

In other embodiments, the acylated catechin polyphenol is a compound of formula (I-f):

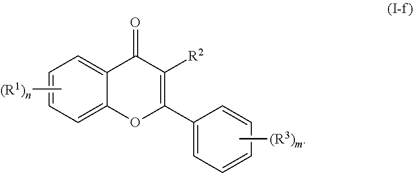

In still further embodiments, n is 2. In certain embodiments, m is 1. In further embodiments, m is 2. In some embodiments, m is 3. In particular embodiments, each R¹ is independently —OR^{A}. In certain embodiments, each R³ is independently H or —OR^{A}. In some embodiments, R² is H or —OR^{A}. In further embodiments, each R^{A} is independently H, optionally substituted alkyl, or a group containing a fatty acid.

In other embodiments, the acylated catechin polyphenol is a compound is of formula (I-e):

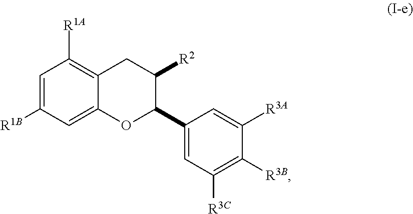

or a pharmaceutically acceptable salt thereof,

where each of R^{1A} and R^{1B} is independently as defined for R¹; and each of R^{3A}, R^{3B}, and R^{3C} is independently as defined for R³.

In yet other embodiments, each of R^{1A} and R^{1B} is independently —OR^{A}. In still other embodiments, each of R^{3A}, R^{3B}, and R^{3C} is independently H, halogen, or —OR^{A}. In some embodiments, R² is a group of formula:

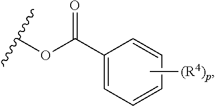

where p is 1, 2, 3, or 4, and each R⁴ is independently selected from the group consisting of H, hydroxy, halogen, a group containing a fatty acid, an optionally substituted alkyl, an optionally substituted alkoxy, a monosaccharide, a sugar acid, phosphate, and sulfate.

In certain embodiments, p is 3. In particular embodiments, each R⁴ is independently H, hydroxy, halogen, a group containing a fatty acid, or an optionally substituted alkoxy. In certain embodiments, R² is a group of formula:

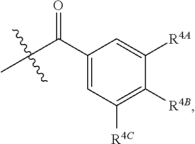

and

each of R^{4A}, R^{4B}, and R^{4C} is as defined for R⁴.

In further embodiments, each of R^{4A}, R^{4B}, and R^{4C} is independently H, hydroxy, halogen, a group containing a fatty acid, or an optionally substituted alkoxy. In yet further embodiments, each R^{A} is independently H, optionally substituted alkyl, fatty acid acyl, or optionally acylated monosaccharide.

In some embodiments, the acylated active agent is an acylated stilbenoid (e.g., the acylated stilbenoid is resveratrol having at least one hydroxyl substituted with a group containing a fatty acid).

In certain embodiments, the acylated active agent is an acylated ellagic acid. In particular embodiments, the acylated active agent is an acylated ellagic acid analogue (e.g., urolithin C having at least one hydroxyl substituted with a group containing a fatty acid).

In particular embodiments, the acylated active agent is an acylated ketone body or pre-ketone body.

In still further embodiments, the acylated active agent includes at least one fatty acid acyl (e.g., a short chain fatty acid acyl). In some embodiments, the short chain fatty acid acyl is acetyl, propionyl, butyryl, or valeryl. In certain embodiments, the short chain fatty acid acyl is acetyl. In particular embodiments, the short chain fatty acid acyl is butyryl.

In another aspect, the invention provides, a composition (e.g., a pharmaceutical composition, nutraceutical composition, food product, food additive, or dietary supplement) including an acylated active agent. In some embodiments, the composition is provided in a unit dosage form

In still other embodiments, the unit dosage form contains at least 0.5 g (e.g., at least 0.7 g, at least 1 g, or at least 2 g) of the acylated active agent. In certain embodiments, the unit dosage form contains 10 g or less (e.g., 9 g or less, 8 g or less, 7 g or less, 6 g or less, 5 g or less) of the acylated active agent. In particular embodiments, the unit dosage form contains 0.5-10 g (e.g., 0.7-10 g, 1-10 g, 2-10 g, 0.5-9 g, 0.7-9 g, 1-9 g, 2-9 g, 0.5-8 g, 0.7-8 g, 1-8 g, 2-8 g, 0.5-7 g, 0.7-7 g, 1-7 g, 2-7 g, 0.5-6 g, 0.7-6 g, 1-6 g, 2-6 g, 0.5-5 g, 0.7-5 g, 1-10 g, or 2-5 g) of the acylated active agent.

In some embodiments, the unit dosage form is a pharmaceutical unit dosage form. In further embodiments, the unit dosage form is a nutraceutical dosage form. In yet further embodiments, the unit dosage form is a serving of a food product.

In other embodiments, the acylated active agent includes a group containing a fatty acid. In yet other embodiments, the group containing a fatty acid is a monosaccharide (e.g., arabinose, xylose, fructose, galactose, glucose, glucosinolate, ribose, tagatose, fucose, and rhamnose), sugar alcohol, or sugar acid having one or more hydroxyl groups substituted with a fatty acid acyl). In still other embodiments, the monosaccharide is L-arabinose, D-xylose, fructose, galactose, D-glucose, glucosinolate, D-ribose, D-tagatose, L-fucose, or L-rhamnose (e.g., the monosaccharide is D-xylose). In further embodiments, the group containing a fatty acid is a fatty acid acyl. In yet further embodiments, the fatty acid is a short chain fatty acid (e.g., acetyl, propionyl, or butyryl). In still further embodiments, the short chain fatty acid is acetyl. In particular embodiments, the short chain fatty acid is butyryl.

In some embodiments, the acylated active agent is an acylated catechin polyphenol. In still further embodiments, the acylated catechin polyphenol is a compound of formula (I):

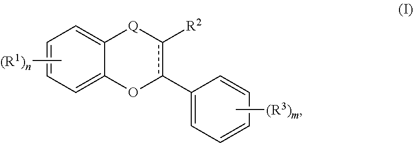

or a pharmaceutically acceptable salt thereof,

where

[symbol] is a single carbon-carbon bond or double carbon-carbon bond;

Q is —CH₂— or —C(O)—;

each R¹ and each R³ is independently H, halogen, —OR^{A}, phosphate, or sulfate;

R² is H or —OR^{A};

each R^{A} is independently H, optionally substituted alkyl, a monosaccharide, a sugar acid, a group containing a fatty acid, or benzoyl optionally substituted with 1, 2, 3, or 4 substituents independently selected from the group consisting of H, hydroxy, halogen, a group containing a fatty acid, an optionally substituted alkyl, an optionally substituted alkoxy, a monosaccharide, a sugar acid, phosphate, and sulfate;

each of n and m is independently 0, 1, 2, 3, or 4.

In some embodiments, at least one R¹ is —OR^{A}, in which R^{A} is a group containing a fatty acid.

In certain embodiments, the acylated catechin polyphenol is a compound is of formula (I-a):

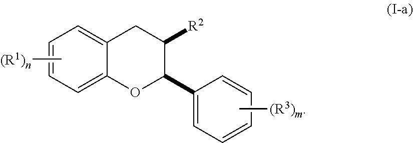

In particular embodiments, the acylated catechin polyphenol is a compound is of formula (I-b):

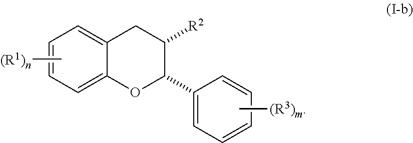

In further embodiments, the acylated catechin polyphenol is a compound is of formula (I-c):

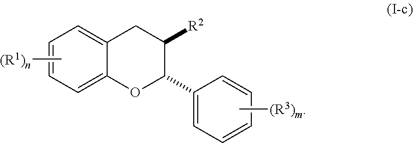

In yet further embodiments, the acylated catechin polyphenol is a compound is of formula (I-d):

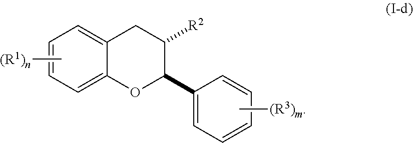

In other embodiments, the acylated catechin polyphenol is a compound of formula (I-f):

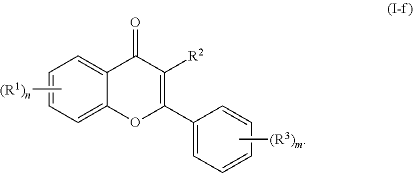

In still further embodiments, n is 2. In certain embodiments, m is 1. In further embodiments, m is 2. In some embodiments, m is 3. In particular embodiments, each R¹ is independently —OR^{A}. In certain embodiments, each R³ is independently H or —OR^{A}. In some embodiments, R² is H or —OR^{A}. In further embodiments, each R^{A} is independently H, optionally substituted alkyl, or a group containing a fatty acid.

In other embodiments, the acylated catechin polyphenol is a compound is of formula (I-e):

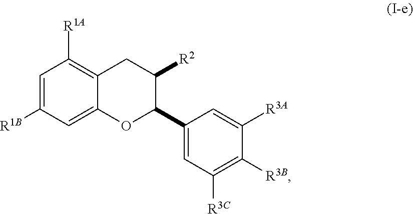

or a pharmaceutically acceptable salt thereof,

where each of R^{1A} and R^{1B} is independently as defined for R¹; and each of R^{3A}, R^{3B}, and R^{3C} is independently as defined for R³.

In yet other embodiments, each of R^{1A} and R^{1B} is independently —OR^{A}. In still other embodiments, each of R^{3A}, R^{3B}, and R^{3C} is independently H, halogen, or —OR^{A}. In some embodiments, R² is a group of formula:

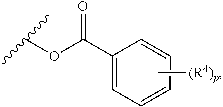

where p is 1, 2, 3, or 4, and each R⁴ is independently selected from the group consisting of H, hydroxy, halogen, a group containing a fatty acid, an optionally substituted alkyl, an optionally substituted alkoxy, a monosaccharide, a sugar acid, phosphate, and sulfate.

In certain embodiments, p is 3. In particular embodiments, each R⁴ is independently H, hydroxy, halogen, a group containing a fatty acid, or an optionally substituted alkoxy. In certain embodiments, R² is a group of formula:

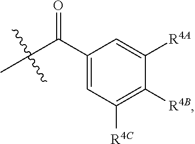

and

each of R^{4A}, R^{4B}, and R^{4C} is as defined for R⁴.

In further embodiments, each of R^{4A}, R^{4B}, and R^{4C} is independently H, hydroxy, halogen, a group containing a fatty acid, or an optionally substituted alkoxy. In yet further embodiments, each R^{A} is independently H, optionally substituted alkyl, fatty acid acyl, or optionally acylated monosaccharide.

In some embodiments, the acylated active agent is an acylated stilbenoid (e.g., the acylated stilbenoid is resveratrol having at least one hydroxyl substituted with a group containing a fatty acid).

In certain embodiments, the acylated active agent is an acylated ellagic acid. In particular embodiments, the acylated active agent is an acylated ellagic acid analogue (e.g., urolithin C having at least one hydroxyl substituted with a group containing a fatty acid).

In still further embodiments, the acylated active agent includes at least one fatty acid acyl (e.g., a short chain fatty acid acyl). In some embodiments, the short chain fatty acid acyl is acetyl, propionyl, or butyryl. In certain embodiments, the short chain fatty acid acyl is acetyl. In particular embodiments, the short chain fatty acid acyl is butyryl.

In another aspect, the invention provides a pharmaceutical composition including an acylated catechin polyphenol, acylated stilbenoid, acylated ellagic acid, acylated ellagic acid analogue, or acylated ketone body or pre-ketone body. In some embodiments, the acylated catechin polyphenol is not a fatty acid peracylated epigallocatechin gallate, fatty acid peracylated gallocatechin gallate, fatty acid peracylated epicatechin gallate, or fatty acid peracylated catechin gallate. In certain embodiments, when the core of the acylated catechin polyphenol is selected from the group consisting of epigallocatechin, epigallocatechin gallate, gallocatechin, gallocatechin gallate, catechin, and catechin gallate, at least one hydroxyl attached to the chromane core is substituted (e.g., with a group containing a fatty acid). In particular embodiments, the pharmaceutical composition includes an acylated catechin polyphenol (e.g., an acylated catechin polyphenol including myricetin or quercetin core). In further embodiments, the pharmaceutical composition includes an acylated stilbenoid (e.g., an acylated stilbenoid including a resveratrol or piceattanol core). In yet further embodiments, the pharmaceutical composition includes an acylated ellagic acid. In still further embodiments, the pharmaceutical composition includes an acylated ellagic acid analogue. In other embodiments, the pharmaceutical composition includes an acylated ketone body or pre-ketone body. In yet other embodiments, the pharmaceutical composition further comprises a pharmaceutically acceptable excipient.

In still another aspect, the invention provides compounds disclosed herein, e.g., those listed in the examples.

### Definitions

The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Non-limiting examples of acyl include fatty acid acyls (e.g., short chain fatty acid acyls (e.g., acetyl)) and benzoyl.

The term “acylated,” as used herein, refers to a group, in which one, two, three, four, or five hydroxyl substituents are independently replaced with acyloxy groups. In a non-limiting example, an acylated monosaccharide is a monosaccharide, in which at least one hydroxyl is replaced with an acyloxy (e.g., a fatty acid acyloxy).

The term “acylated catechin polyphenol,” as used herein, represents a substituted compound having the core of formula (A):

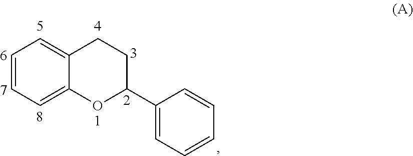 [SMILES: c1ccc(C2CCc3ccccc3O2)cc1]

or a multimer thereof, or a salt thereof,

where the substituents are independently selected from the group consisting of —OR^{A}, —OCOO—R^{A}, —NHR^{B}, oxo, halogen, optionally substituted C₁₋₂₀ alkyl, optionally substituted C₂₋₂₀ alkenyl, optionally substituted thioalkyl, optionally substituted alkylsulfonyl, optionally substituted alkylsulfenyl, optionally substituted alkylsulfinyl, optionally substituted thioaryl, optionally substituted aryl thioalkyl, optionally substituted thioalkenyl, dialkylamino, sulfate, phosphate, ascorbic acid, optionally substituted heterocyclyl, nitro, amino acids, C₁₋₆ esters of amino acids, optionally acylated monosaccharide, and optionally acylated sugar acid, where each R^{A} is independently H, optionally substituted alkyl, a group containing a fatty acid, or benzoyl optionally substituted with one, two, three, or four substituents independently selected from the group consisting of H, hydroxyl, halogen, a group containing a fatty acid, optionally substituted alkoxy, and optionally substituted alkyl, and where R^{B} is independently H or optionally substituted alkyl;

where the carbon-carbon bond connecting carbon 2 and carbon 3 in formula (A) is a single bond or a double bond;

where the multimer includes a total of 2 or 3 cores of formula (A), each core substituted independently as described above; and

where two vicinal centers in core (A) may be further substituted with a group —(O)_{q}-L¹-L²-, where q is 0 or 1, L¹ is optionally substituted alkylene, optionally substituted alkenylene, or optionally substituted heterocyclylene; and L² is a covalent bond, optionally substituted heterocyclylene, or optionally substituted cycloalkylene;

provided that at least one of positions 5, 6, 7, and 8 is —OR^{A}, where R^{A} is a group containing a fatty acid or a benzoyl optionally substituted with one, two, three, or four substituents independently selected from the group consisting of H, hydroxyl, a halogen, a group containing a fatty acid, an optionally substituted alkoxy, and an optionally substituted alkyl; and

provided that the substituted compound includes at least one group containing a fatty acid.

The term “acylated catechin polyphenol” also refers to a compound of formula (I):

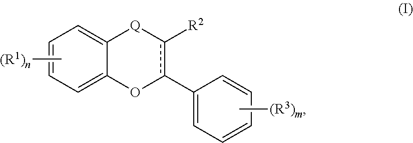

or a pharmaceutically acceptable salt thereof,
where

[symbol] is a single carbon-carbon bond or double carbon-carbon bond;

Q is —CH₂— or —C(O)—;

each R¹ and each R³ is independently H, halogen, —OR^{A}, phosphate, or sulfate;

R² is H or —OR^{A};

each R^{A} is independently H, optionally substituted alkyl, a monosaccharide, a sugar acid, a group containing a fatty acid, or benzoyl optionally substituted with 1, 2, 3, or 4 substituents independently selected from the group consisting of H, hydroxy, halogen, a group containing a fatty acid, an optionally substituted alkyl, an optionally substituted alkoxy, a monosaccharide, a sugar acid, phosphate, and sulfate; and

each of n and m is independently 0, 1, 2, 3, or 4.

The term “acylated ketone body or pre-ketone body,” as used herein, represents a ketone body or pre-ketone body having one or more hydroxyls substituted with alkyl, acyl, or a group containing a fatty acid. Preferably, the acylated ketone body or pre-ketone body includes at least one group containing a fatty acid.

The term “acylated stilbenoid,” as used herein, represents a stilbenoid, in which one, two, three, four, or five hydroxyl groups are independently replaced with a substituent —OR, where each R is independently selected from the group consisting of an acyl, alkyl, and group including a fatty acid, provided that at least one R is a group including a fatty acid.

The term “acylated ellagic acid,” as used herein, represents compounds of the following structures:

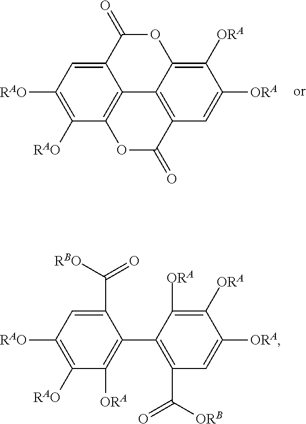

or a salt thereof,

where each R^{A} is independently H, alkyl, acyl, or a group containing a fatty acid; and each R^{B} is independently H, alkyl, or a group containing a fatty acid; provided that at least one R^{A} and/or at least one R^{B}, when present, is a group containing a fatty acid acyl.

The term “acylated ellagic acid analogue,” as used herein, represents compounds of the following structure:

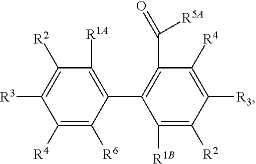

or a salt thereof,
where

each of R², R³, and R⁴ is independently H or —OR^{A};

R⁶ is H or —(CO)—R^{5B};

R^{1A} is H or —OR^{A}, and R^{5A} is —OH or —ORB; or R^{1A} and R^{5A} combine to form —O—;

R^{1B} is H or —OR^{A}, and R^{5B} is absent, —OH, or —ORB; or R^{1B} and R^{5B} combine to form —O—;

each R^{A} is independently H, O-protecting group, alkyl, acyl, or a group containing a fatty acid;

each R^{B} is independently H, O-protecting group, alkyl, or a group containing a fatty acid;

provided that at least one R^{A} and/or at least one R^{B} is a group containing a fatty acid.

The term “acyloxy,” as used herein, represents a chemical substituent of formula —OR, where R is acyl. An optionally substituted acyloxy is an acyloxy that is optionally substituted as described herein for acyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alcohol oxygen atom,” as used herein, refers to a divalent oxygen atom, where one valency of the alcohol oxygen atom is bonded to a first carbon atom, and another valency is bonded to a second carbon atom, where the first carbon atom is an spa-hybridized carbon atom, and the second carbon atom is an spa-hybridized carbon atom or an sp²-hybridized carbon atom of a carbonyl group.

The term “aldonyl,” as used herein, refers to a monovalent substituent that is aldonic acid in which a carboxylate hydroxyl is replaced with a valency.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkenyl,” as used herein, represents acyclic monovalent straight or branched chain hydrocarbon groups containing one, two, or three carbon-carbon double bonds. Alkenyl, when unsubstituted, has from 2 to 22 carbons, unless otherwise specified. In certain preferred embodiments, alkenyl, when unsubstituted, has from 2 to 12 carbon atoms (e.g., 1 to 8 carbons). Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkyl,” as used herein, refers to an acyclic straight or branched chain saturated hydrocarbon group, which, when unsubstituted, has from 1 to 22 carbons (e.g., 1 to 20 carbons), unless otherwise specified. In certain preferred embodiments, alkyl, when unsubstituted, has from 1 to 12 carbons (e.g., 1 to 8 carbons). Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; alkylsulfenyl; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; cyano; ═O; ═S; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkenylene,” as used herein, refers to a straight or branched chain alkenyl group with one hydrogen removed, thereby rendering this group divalent. Non-limiting examples of the alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. An optionally substituted alkenylene is an alkenylene that is optionally substituted as described herein for alkyl.

The term “alkylene,” as used herein, refers to a saturated divalent hydrocarbon group that is a straight or branched chain saturated hydrocarbon, in which two valencies replace two hydrogen atoms. Non-limiting examples of the alkylene group include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. An optionally substituted alkylene is an alkylene that is optionally substituted as described herein for alkyl.

The term “alkylsulfenyl,” as used herein, represents a group of formula —S-(alkyl). An optionally substituted alkylsulfenyl is an alkylsulfenyl that is optionally substituted as described herein for alkyl.

The term “alkylsulfinyl,” as used herein, represents a group of formula —S(O)-(alkyl). An optionally substituted alkylsulfinyl is an alkylsulfinyl that is optionally substituted as described herein for alkyl.

The term “alkylsulfonyl,” as used herein, represents a group of formula —S(O)₂-(alkyl). An optionally substituted alkylsulfonyl is an alkylsulfonyl that is optionally substituted as described herein for alkyl.

The term “amino acid,” as used herein, represents proline, taurine, or a compound having an amino group and a carboxylate or sulfonate group separated by an optionally substituted alkylene or optionally substituted arylene. Amino acids are small molecules and have a molecular weight of <900 g/mol (preferably, <500 g/mol). Preferably, when the linker is alkylene, the linker may be optionally substituted as described herein for alkyl. In some embodiments, optionally substituted alkylene is an alkylene substituted with 1 or 2 groups that are independently hydroxyl, thiol, amino, guanidine, carbamoylamino, imidazolyl, indolyl, -SeH, oxo, 4-hydroxyphenyl, phenyl, or -SMe. Non-limiting examples of amino acids include alanine, arginine, asparagine, aspartic acid, cysteine, glutamic acid, glutamine, glycine, histidine, isoleucine, leucine, lysine, methionine, phenylalanine, proline, selenocysteine, serine, threonine, tyrosine, tryptophan, ornithine, citrulline, aminobenzoic acid, and taurine.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. An optionally substituted aryl alkyl is an aryl alkyl, in which aryl and alkyl portions may be optionally substituted as the individual groups as described herein.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “cancer,” as used herein, refers to a group of proliferative diseases characterized by uncontrolled division of abnormal cells in a subject. The cancer may be a solid tumor or a non-solid (e.g., hematologic) cancer. Non-limiting examples of cancers include stomach cancer, skin cancer, lung cancer, breast cancer, non-Hodgkin lymphoma, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, colorectal cancer, small intestine cancer, acute lymphoblastic leukemia, acute myeloid leukemia, adrenocortical carcinoma, Kaposi sarcoma, primary CNS lymphoma, anal cancer, astrocytoma, glioblastoma, bladder cancer, Ewing sarcoma, osteosarcoma, non-Hodgkin lymphoma, breast cancer, brain tumor, cervical cancer, bile duct cancer, chronic lymphocytic leukemia, chronic myelogenous leukemia, gallbladder cancer, gastrointestinal stromal tumor, ovarian cancer, testicular cancer, multiple myeloma, neuroblastoma, pancreatic cancer, parathyroid cancer, prostate cancer, rectal cancer, and Wilms tumor.

The term “cancer marker,” as used herein, is an observable indication of the presence, absence, or risk of a cancer (e.g., stomach cancer, skin cancer, lung cancer, breast cancer, non-Hodgkin lymphoma, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, or colorectal cancer). The level of a cancer marker may directly or inversely correlate with a cancer state. Non-limiting examples of the cancer markers are a CD4⁺CD25⁺ Treg cell (e.g., CD4⁺CD25⁺Foxp3⁺ Treg cell) count, cytotoxic T cell count, Tₕ1 cell count, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, and 8-iso-prostaglandin F_{2α} (8-iso-PGF2α) level. The cancer markers may be measured using methods known in the art. For example, blood sample analyses may be performed to measure a CD4⁺CD25⁺ Treg cell (e.g., CD4⁺CD25⁺Foxp3⁺ Treg cell) count, cytotoxic T cell count, Tₕ1 level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, and 8-iso-prostaglandin F_{2α} (8-iso-PGF2α) level.

The term “carboxylate,” as used herein, represents group —COOH or a salt thereof.

The term “catechin polyphenol,” as used herein, refers to a compound of formula:

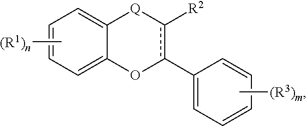

where

[symbol] is a single carbon-carbon bond or double carbon-carbon bond;

Q is —CH₂— or —C(O)—;

each R¹ and each R³ is independently H, halogen, —OR^{A}, phosphate, or sulfate;

R² is H or —OR^{A};

each R^{A} is independently H, optionally substituted alkyl, a monosaccharide, a sugar acid, or benzoyl optionally substituted with 1, 2, 3, or 4 substituents independently selected from the group consisting of H, hydroxy, halogen, optionally substituted alkyl, optionally substituted alkoxy, monosaccharide, sugar acid, phosphate, and sulfate; and each of n and m is independently 1, 2, 3, or 4.

Non-limiting examples of catechin polyphenols include epigallocatechin gallate, epigallocatechin, quercetin, myricetin, luteolin, and apigenin. When a catechin polyphenol is acylated, one or more of the hydroxyl groups in the catechin polyphenol (e.g., epigallocatechin gallate, epigallocatechin, myricetin, quercetin, luteolin, or apigenin) are independently substituted with a group containing a fatty acid.

The term “chromane core,” as used herein, refers to the following group:

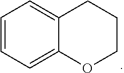

When the chromane core is part of a compound (e.g., catechin polyphenol), the carbon atoms of the chromane core are substituted as required by the structure of such a compound.

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1]heptyl, 2-bicyclo[2.2.1]heptyl, 5-bicyclo[2.2.1]heptyl, 7-bicyclo[2.2.1]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkoxy; acyloxy; alkylsulfenyl; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent group that is a cycloalkyl group, in which one hydrogen atom is replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. An optionally substituted cycloalkoxy is cycloalkoxy that is optionally substituted as described herein for cycloalkyl.

The term “dialkylamino,” as used herein, refers to a group —NR₂, where each R is independently alkyl.

The terms “ellagic acid” and “ellagic acid analogue,” as used herein, collectively refer to a compound of the structure:

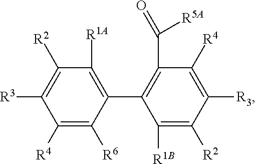

where

each of R², R³, and R⁴ is independently H or —OR^{A};

R⁶ is H or —(CO)—R^{5B};

R^{1A} is H or —OR^{A}, and R^{5A} is —OH or —OR^{A}; or R^{1A} and R^{5A} combine to form —O—;

R^{1B} is H or —OR^{A}, and R^{5B} is absent, —OH, or —OR^{A}; or R^{1B} and R^{5B} combine to form —O—;

each R^{A} is independently H or O-protecting group.

When the ellagic acid or its analogue is present in an acylated ellagic acid or an acylated ellagic acid analogue, from one to all hydroxyls in the ellagic acid or its analogue are substituted with a group containing a fatty acid. The term “ellagic acid analogue,” refers to the compounds and groups of the above structure that are not ellagic acid. The term “ellagic acid” refers to the following two compounds:

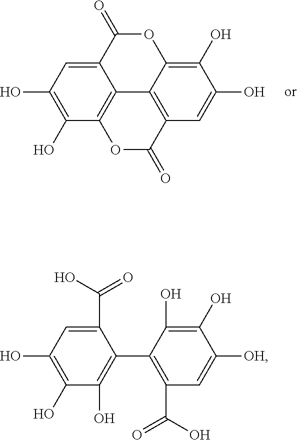 [SMILES: O=c1oc2c(O)c(O)cc3c(=O)oc4c(O)c(O)cc1c4c23 ; O=C(O)c1cc(O)c(O)c(O)c1-c1c(C(=O)O)cc(O)c(O)c1O]

or these compounds within the structure of a conjugate.

Non-limiting examples of ellagic acid analogues include urolithin A, urolithin B, urolithin C, urolithin D, urolithin E, and urolithin M5.

The term “ester bond,” as used herein, refers to a covalent bond between an alcohol or phenolic oxygen atom and a carbonyl group that is further bonded to a carbon atom.

The term “fatty acid,” as used herein, refers to a short-chain fatty acid, a medium chain fatty acid, a long chain fatty acid, a very long chain fatty acid, or an unsaturated analogue thereof, or a phenyl-substituted analogue thereof. Short chain fatty acids contain from 1 to 6 carbon atoms, medium chain fatty acids contain from 7 to 13 carbon atoms, and a long-chain fatty acids contain from 14 to 22 carbon atoms. A fatty acid may be saturated or unsaturated. An unsaturated fatty acid includes 1, 2, 3, 4, 5, or 6 carbon-carbon double bonds. Preferably, the carbon-carbon double bonds in unsaturated fatty acids have Z stereochemistry.

The term “fatty acid acyl,” as used herein, refers to a fatty acid, in which the hydroxyl group is replaced with a valency.

The term “fatty acid acyloxy,” as used herein, refers to group —OR, where R is a fatty acid acyl.

The term “group containing a fatty acid,” as used herein, represents a monovalent substituent including at least one fatty acid within its structure and having the valency on a carbon atom of a carbonyl group or on an anomeric carbon atom. A group containing a fatty acid bonds to a core through a carbonate linker, carbamate linker, ester bond, glycosidic bond, or amide bond. A group containing a fatty acid may be a group selected from the group consisting of monosaccharide, ketone body, pre-ketone body, aldonyl, uronyl, ulosonyl, and fatty acid acyl, and where at least one (preferably, each available) hydroxyl in the monosaccharide, ketone body, pre-ketone body, aldonyl, uronyl, and ulosonyl is optionally and independently substituted with a fatty acid acyl.

The term “halogen,” as used herein, represents a halogen selected from bromine, chlorine, iodine, and fluorine.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, quinazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkenyl; alkoxy; acyloxy; aryloxy; alkylsulfenyl; alkylsulfinyl; alkylsulfonyl; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic non-aromatic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidinyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkoxy; acyloxy; alkylsulfenyl; alkylsulfinyl; alkylsulfonyl; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as the described for heterocyclyl and alkyl, respectively.

The term “heterocyclylene,” as used herein, represents a heterocyclyl, in which one hydrogen atom is replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH.

The term “ketone body,” as used herein, refers to (i) β-hydroxybutyric acid, or (ii) a group that is β-hydroxybutyric acid, where at least one hydroxyl hydrogen atom is replaced with a valency or a carboxylate —OH is replaced with a valency.

The term “ketone body acyl,” as used herein, refers to a β-hydroxybutyric acid, in which the carboxylate —OH group is replaced with a valency.

The term “modulating,” as used herein, refers to an observable change in the level of a marker in a subject, as measured using techniques and methods known in the art for the measurement of the marker. Modulating the marker level in a subject may result in a change of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In some embodiments, modulating is increasing the level of a marker in a subject. Increasing the marker level in a subject may result in an increase of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In other embodiments, modulating is decreasing the level of a marker in a subject. Decreasing the marker level in a subject may result in a decrease of at least 1% relative to prior to administration (e.g., at least 5%, 10%, 15%, 20%, 25%, 30%, 35%, 40%, 45%, 50%, 55%, 60%, 65%, 70%, 75%, 80%, 85%, 90%, 95% or at least 98% or more relative to prior to administration; e.g., up to 100% relative to prior to administration). In embodiments in which a parameter is increased or decreased (or reduced) in a subject following a step of administering a composition described herein, the increase or decrease may take place and/or be detectable within a range of time following the administration (e.g., within six hours, 24 hours, 3 days, a week or longer), and may take place and/or be detectable after one or more administrations (e.g., after 2, 3, 4, 5, 6, 7, 8, 9, 10 or more administrations, e.g., as part of a dosing regimen for the subject).

The term “oxo,” as used herein, represents a divalent oxygen atom (e.g., the structure of oxo may be shown as ═O).

The term “peracylated,” as used herein, refers to a core, in which all hydroxyls are substituted with acyls. For example, a fatty acid peracylated catechin polyphenol is a catechin polyphenol, in which all hydroxyls are substituted with fatty acid acyls. Non-limiting examples of a fatty acid peracylated catechin polyphenols are epigallocatechin gallate octaacetate and epigallocatechin gallate octabutyrate.

The term “phenolic oxygen atom,” as used herein, refers to a divalent oxygen atom within the structure of a compound, where one valency of the phenolic oxygen atom is bonded to a first carbon atom, and another valency is bonded to a second carbon atom, where the first carbon atom is an sp²-hybridized carbon atom within a benzene ring, and the second carbon atom is an spa-hybridized carbon atom or an sp²-hybridized carbon atom.

The term “phosphate, as used herein, represents group —OPO(OH)₂ or a salt thereof.

The term “pre-ketone body,” as used herein, represents (i) a ketone body having hydroxymethyl instead of a carboxylate, or (ii) a group that is a ketone body having hydroxymethyl instead of a carboxylate, where at least one hydroxyl is replaced with —OR, where R is a valency. A non-limiting example of a pre-ketone body is butane-1,3-diol or 4-hydroxybutan-2-one.

The term “pre-ketone body acyl,” as used herein, refers to a pre-ketone body, in which the carboxylate —OH group is replaced with a valency.

The term “protecting group,” as used herein, represents a group intended to protect a hydroxy, an amino, or a carbonyl from participating in one or more undesirable reactions during chemical synthesis.

The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylphenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “stilbenoid,” as used herein, represents a trans-stilbene that, when not acylated, is substituted with one, two, three, four, or five substituents independently selected from the group consisting of alkoxy (e.g., methoxy) and hydroxyl. Non-limiting examples of stilbenoids include resveratrol, pterostilbene, rhapontigenin, pinostilbene, oxyresveratrol, 4-methoxyresveratrol, and piceatannol. When the stilbenoid is acylated, from one to all of the hydroxyl groups in the stilbenoid is independently substituted with a group including a fatty acid acyl.

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. Non-limiting examples of diseases, disorders, and conditions include cancer, as described herein.

The term “sugar acid,” as used herein, refers to a monosaccharide, in the linear form of which, one or both terminal positions are oxidized to a carboxylic acid. There are four classes of sugar acids: aldonic acid, ulosonic acid, uronic acid, and aldaric acid. Any of the four sugar acid classes may be used in acylated active agents disclosed herein. Non-limiting examples of sugar acids include gluconic acid.

The term “sulfate,” as used herein, represents group —OSO₃H or a salt thereof.

The term “thioalkenyl,” as used herein, represents a group —SR, where R is alkenyl. An optionally substituted thioalkenyl is thioalkenyl that is optionally substituted as described herein for alkenyl.

The term “thioalkyl,” as used herein, represents a group —SR, where R is alkyl. An optionally substituted thioalkyl is thioalkyl that is optionally substituted as described herein for alkyl.

The term “thioaryl,” as used herein, represents a group —SR, where R is aryl. An optionally substituted thioaryl is thioaryl that is optionally substituted as described herein for aryl.

“Treatment” and “treating,” as used herein, refer to the medical management of a subject with the intent to improve, ameliorate, stabilize, prevent or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “ulosonyl,” as used herein, refers to a monovalent substituent that is a ulosonic acid in which a carboxylate hydroxyl is replaced with a valency.

The term “uronyl,” as used herein, refers to a monovalent substituent that is a uronic acid in which a carboxylate hydroxyl is replaced with a valency.

The term “vitamin E,” as used herein, refers to tocopherols and tocotrienols. Vitamin E may be a compound of the following structure:

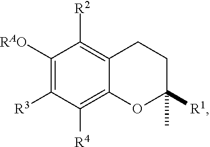

where

R¹ is

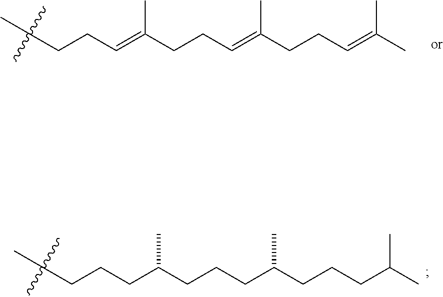 [SMILES: *CC/C=C(\C)CC/C=C(\C)CCC=C(C)C ; *CCC[C@H](C)CCC[C@H](C)CCCC(C)C]

each of R², R³, and R⁴ is independently H or Me; and

R^{A} is H or a group containing a fatty acid.

When the vitamin E is present in an acylated vitamin E, R^{A} is a group containing a fatty acid.

The compounds described herein, unless otherwise noted, encompass isotopically enriched compounds (e.g., deuterated compounds), tautomers, and all stereoisomers and conformers (e.g. enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

### BRIEF DESCRIPTION OF THE DRAWINGS

FIG. is a chart showing the CD4⁺CD25⁺ cell counts as a percentage of all CD4⁺ T cells in seven animal cohorts: (1) untreated animals receiving a normal diet, (2) untreated animals receiving a high-fat diet, (3) animals receiving acetate along with a high-fat diet, (4) animals receiving epigallocatechin gallate (EGCG) along with a high-fat diet, (5) animals receiving epigallocatechin gallate (EGCG) and acetate as different compounds along with a high-fat diet, (6) animals receiving epigallocatechin gallate octaacetate (EGCG-8A) along with a high-fat diet, and (7) animals receiving rosiglitazone along with a high-fat diet.

### DETAILED DESCRIPTION

The invention provides acylated active agents and methods for modulating a cancer marker in a subject or treating cancer in a subject. Without wishing to be bound by theory, the disclosed acylated active agents are believed to act in concert with, or in lieu of, the microbiota of a subject to modulate, for example, the host's immune system.

It has been surprisingly found that administration of an acylated active agent (e.g., epigallocatechin-3-gallate octaacetate) to a subject can induce Treg differentiation (e.g., CD4⁺CD25⁺ Treg differentiation) and thus can produce beneficial effects in subjects suffering from a cancer. Surprisingly, administration of an acylated catechin polyphenol was found to produce superior activity relative to the administration of the same dose of acylated catechin polyphenol components as separate compounds.

It has also been surprisingly found that certain acylated active agent components can reduce cancer cell viability (e.g., resveratrol, epigallocatechin-3-gallate, urolithin C, epigallocatechin, myricetin, luteolin, apigenin, butyrate, propionate, or valerate). Administration of these agents to cancer tissue in the acylated active agent form can produce superior anti-cancer effect relative to administration of separate components.

The components of the acylated active agent (e.g., components of acylated catechin polyphenol, acylated stilbenoid, acylated ellagic acid, acylated ellagic acid analogue, acylated ketone body or pre-ketone body, or acylated vitamin) may act synergistically to modulate a cancer marker, e.g., upon hydrolysis in the GI tract of the subject receiving the acylated active agent. The components of the acylated active agent (e.g., components of acylated catechin polyphenol, acylated stilbenoid, acylated ellagic acid, acylated ellagic acid analogue, acylated ketone body or pre-ketone body, or acylated vitamin) may act synergistically to treat cancer, e.g., upon hydrolysis in the GI tract of the subject receiving the acylated active agent.

Advantageously, acylated active agents disclosed herein may have superior organoleptic properties (e.g., palatability). This provides an important advantage as the individual components (e.g., fatty acid (e.g., propionic acid, butyric acid, or valeric acid), catechin polyphenol (e.g., epigallocatechin gallate, myricetin, apigenin, or luteolin), stilbenoid (e.g., resveratrol), ellagic acid, ellagic acid analogue (e.g., urolithin C), ketone body (e.g., β-hydroxybutyrate), pre-ketone body (e.g., 1,3-butanediol), or vitamin (e.g., vitamin E)) may exhibit less desirable organoleptic properties (e.g., palatability). Improved organoleptic properties facilitate oral administration, and are particularly advantageous for delivery of high unit dosages (e.g., unit dosages of 0.5 g or higher).

Acylated Active Agents

An acylated active agent disclosed herein may be, for example, an acylated catechin polyphenol, acylated stilbenoid, acylated ellagic acid, acylated ellagic acid analogue, acylated ketone body or pre-ketone body, or acylated vitamin (e.g., acylated vitamin E).

Typically, an acylated catechin polyphenol includes a core of formula (A) (e.g., a catechin polyphenol core) linked to at least one acyl group (e.g., fatty acid acyl) through ester bond(s), amide bond(s), carbonate linker(s), carbamate linker(s), and/or glycosidic bond(s). For example, an acylated active agent may include a catechin polyphenol substituted with one or more substituents independently selected from the group consisting of an alkyl, acyl, and group containing a fatty acid (e.g., a short chain fatty acid or a medium chain fatty acid). The fatty acid may be, e.g., a short chain fatty acid (e.g., acetyl, propionyl, or butyryl). In some embodiments, the short chain fatty acid is acetyl. In particular embodiments, the short chain fatty acid is butyryl.

An acylated active agent disclosed herein may include, e.g., at least one group containing a fatty acid. A group containing a fatty acid may be, e.g., a fatty acid (e.g., short chain fatty acid or medium chain fatty acid), a monosaccharide having one or more hydroxyl groups substituted with fatty acid acyls (e.g., short chain fatty acid acyls or medium chain fatty acid acyls), a sugar acid (e.g., aldonic acid) having one or more alcohol hydroxyl groups substituted with fatty acid acyls (e.g., short chain fatty acid acyls or medium chain fatty acid acyls), or a sugar alcohol having one or more alcohol hydroxyl groups substituted with fatty acid acyls (e.g., short chain fatty acid acyls or medium chain fatty acid acyls). A monosaccharide may be, e.g., arabinose, xylose, fructose, galactose, glucose, glucosinolate, ribose, tagatose, fucose, or rhamnose. In some embodiments, the monosaccharide is L-arabinose, D-xylose, fructose, galactose, D-glucose, glucosinolate, D-ribose, D-tagatose, L-fucose, or L-rhamnose (e.g., the monosaccharide is D-xylose). The group containing a fatty acid may be, e.g., a fatty acid acyl. A sugar acid may be, e.g., aldonic acid, ulosonic acid, uronic acid, or aldaric acid. A sugar acid may be, e.g., xylonic acid, gluconic acid, glucuronic acid, galacturonic acid, tartaric acid, saccharic acid, or mucic acid. A sugar alcohol may be, e.g., glycerol, erythritol, threitol, arabitol, xylitol, tibitol, mannitol, sorbitol, galactitol, fucitol, iditol, or inositol.

In certain embodiments, the group may be a monovalent group of the following formula:

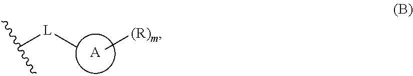

where

L is absent or carbonate linker;

group A is a fatty acid acyl, ketone body, pre-ketone body, monosaccharide, sugar acid, or sugar alcohol;

each R is independently ketone body optionally having a hydroxyl group that is optionally substituted with an acyl (e.g., a fatty acid acyl), pre-ketone body optionally having a hydroxyl group that is optionally substituted with an acyl (e.g., a fatty acid acyl), or acyl (e.g., a fatty acid acyl); and

m is an integer from 0 to the total number of available hydroxyl groups in group A (e.g., 1, 2, 3, 4, or 5);

provided that

L is a carbonate linker, if group A has a valency on a non-glycosidic alcohol oxygen atom, and L is attached to an alcohol or phenolic oxygen atom in the catechin polyphenol, stilbenoid, ellagic acid, or ellagic acid analogue; and

L is absent, if group A has a valency on a carbonyl carbon atom, and L is attached to an alcohol or phenolic oxygen atom in the catechin polyphenol, stilbenoid, ellagic acid, or ellagic acid analogue; or

L is absent, if group A has a valency on an oxygen atom, and L is attached to a carbonyl carbon atom in the ellagic acid or ellagic acid analogue.

In certain embodiments, the group of formula (B) includes at least one fatty acid acyl.

In some embodiments, the fatty acid(s) are short chain fatty acid acyls (e.g., butyryls). In particular embodiments, the fatty acid(s) in the group containing a fatty acid are medium chain fatty acid acyls (e.g., octanoyl).

Non-limiting examples of a group containing a fatty acid are:

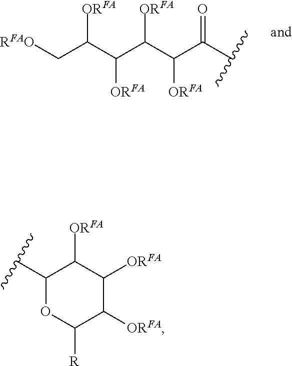

where

R is H, —CH₃, or —CH₂OR^{FA};

each R^{FA} is independently H or a fatty acid acyl (e.g., a short chain fatty acid acyl or medium chain fatty acid acyl);

provided that at least one R^{FA} is a fatty acid acyl (e.g., a short chain fatty acid acyl).

Acylated Catechin Polyphenols

An acylated catechin polyphenol of the invention may be a substituted compound having the core of formula (A):

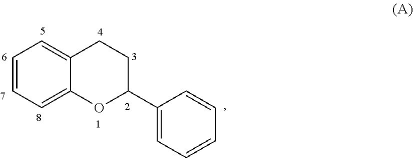 [SMILES: c1ccc(C2CCc3ccccc3O2)cc1]

or a multimer thereof, or a salt thereof,

where the substituents are independently selected from the group consisting of —OR^{A}, —OCOO—R^{A}, —NHR^{B}, oxo, halogen, optionally substituted C₁₋₂₀ alkyl, optionally substituted C₂₋₂₀ alkenyl, optionally substituted thioalkyl, optionally substituted alkylsulfonyl, optionally substituted alkylsulfenyl, optionally substituted alkylsulfinyl, optionally substituted thioaryl, optionally substituted aryl thioalkyl, optionally substituted thioalkenyl, dialkylamino, sulfate, phosphate, ascorbic acid, optionally substituted heterocyclyl, nitro, amino acids, C₁₋₆ esters of amino acids, optionally acylated monosaccharide, and optionally acylated sugar acid, where each R^{A} is independently H, optionally substituted alkyl, a group containing a fatty acid, or benzoyl optionally substituted with one, two, three, or four substituents independently selected from the group consisting of H, hydroxyl, halogen, a group containing a fatty acid, optionally substituted alkoxy, and optionally substituted alkyl, and where R^{B} is independently H or optionally substituted alkyl;

where the carbon-carbon bond connecting carbon 2 and carbon 3 in formula (A) is a single bond or a double bond;

where the multimer includes a total of 2 or 3 cores of formula (A), each core substituted independently as described above; and

where two vicinal centers in core (A) may be further substituted with a group —(O)_{q}-L¹-L²-, where q is 0 or 1, L¹ is optionally substituted alkylene, optionally substituted alkenylene, or optionally substituted heterocyclylene; and L² is a covalent bond, optionally substituted heterocyclylene, or optionally substituted cycloalkylene.

In some embodiments, at least one of positions 5, 6, 7, and 8 is —OR^{A}, where R^{A} is a group containing a fatty acid or benzoyl optionally substituted with one, two, three, or four substituents independently selected from the group consisting of H, hydroxyl, halogen, a group containing a fatty acid, optionally substituted alkoxy, and optionally substituted alkyl. In some embodiments, the compound of formula (A) includes at least one group containing a fatty acid.

An acylated catechin polyphenol of the invention may be a catechin polyphenol, in which one or more hydroxyl groups are independently replaced with —OR, where each R is independently selected from the group consisting of an acyl, alkyl, and group containing a fatty acid. In some embodiments, at least one R is a group containing a fatty acid.

An acylated catechin polyphenol may be a compound of formula (I):

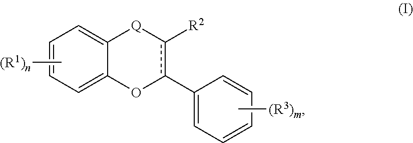

or a pharmaceutically acceptable salt thereof,
where [symbol]
is a single carbon-carbon bond or double carbon-carbon bond;

Q is —CH₂— or —C(O)—;

each R¹ and each R³ is independently H, halogen, —OR^{A}, phosphate, or sulfate;

R² is H or —OR^{A};

each R^{A} is independently H, optionally substituted alkyl, a monosaccharide, a monosaccharide, a sugar acid, a group containing a fatty acid, or benzoyl optionally substituted with 1, 2, 3, or 4 substituents independently selected from the group consisting of H, hydroxy, halogen, a group containing a fatty acid, an optionally substituted alkyl, an optionally substituted alkoxy, a monosaccharide, a sugar acid, phosphate, and sulfate; and

each of n and m is independently 0, 1, 2, 3, or 4.

In some embodiments, the compound includes at least one group containing a fatty acid. In particular embodiments, at least one R¹ is —OR^{A}, in which R^{A} is a group containing a fatty acid.

In particular embodiments, [symbol] is a single carbon-carbon bond. In certain embodiments, Q is —CH₂—.

In some embodiments, the acylated catechin polyphenol is of formula (I-a):

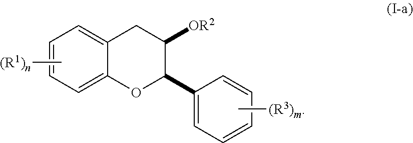

In certain embodiments, the acylated catechin polyphenol is of formula (I-b):

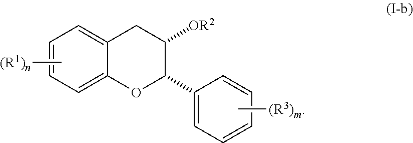

In particular embodiments, the acylated catechin polyphenol is of formula (I-c):

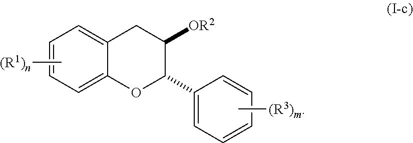

In further embodiments, the acylated catechin polyphenol is of formula (I-d):

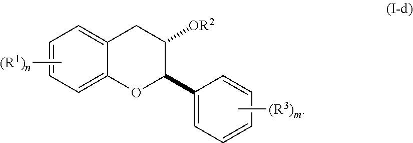

In other embodiments, the acylated catechin polyphenol is a compound of formula (I-f):

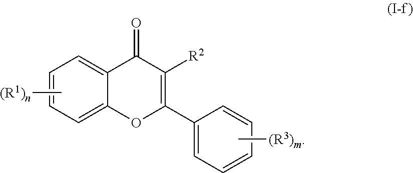

In some embodiments, n is 2. In certain embodiments, m is 1. In further embodiments, m is 2. In particular embodiments, m is 3. In certain embodiments, each R¹ is independently —OR^{A}. In further embodiments, each R³ is independently H or —OR^{A}. In some embodiments, R² is H or —OR^{A}. In further embodiments, each R^{A} is independently H, optionally substituted alkyl, or a group containing a fatty acid.

In some embodiments, R² is a group of formula:

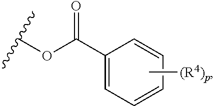

where p is 1, 2, 3, or 4, and each R⁴ is independently selected from the group consisting of H, hydroxy, halogen, a group containing a fatty acid, an optionally substituted alkyl, an optionally substituted alkoxy, a monosaccharide, a sugar acid, phosphate, and sulfate.

In certain embodiments, p is 3. In particular embodiments, R⁴ is independently H, hydroxy, halogen, a group containing a fatty acid, or an optionally substituted alkoxy.

In some embodiments, the acylated catechin polyphenol is of formula (I-e):

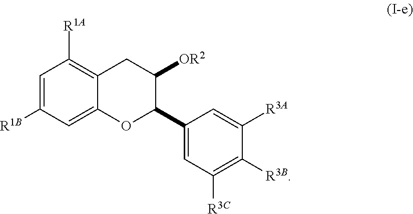

In certain embodiments, each of R^{1A} and R^{1B} is independently —OR^{A}. In particular embodiments, each of R^{3A}, R^{3B}, and R^{3C} is independently H, halogen, or —OR^{A}.

In further embodiments, R² is a group of formula:

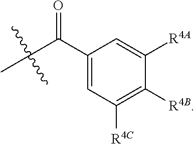

In yet further embodiments, R^{4A}, R^{4B}, and R^{4C} is independently H, hydroxy, halogen, a group containing a fatty acid, or an optionally substituted alkoxy.

In some embodiments, each R^{A} is independently H, optionally substituted alkyl, fatty acid acyl, or optionally acylated monosaccharide.

In certain embodiments, the acylated catechin polyphenol includes at least one fatty acid acyl (e.g., a short chain fatty acid acyl (e.g., the short chain fatty acid acyl is acetyl, propionyl, or butyryl)).

Acylated Ellagic Acid and Acylated Ellagic Acid Analogues

An acylated ellagic acid includes an ellagic acid core having one or more hydroxyls substituted with an acyl (e.g., a fatty acid acyl). An acylated ellagic acid analogue includes an ellagic acid analogue core having one or more hydroxyls substituted with an acyl (e.g., a fatty acid acyl).

An acylated ellagic acid is a compound of the following structures:

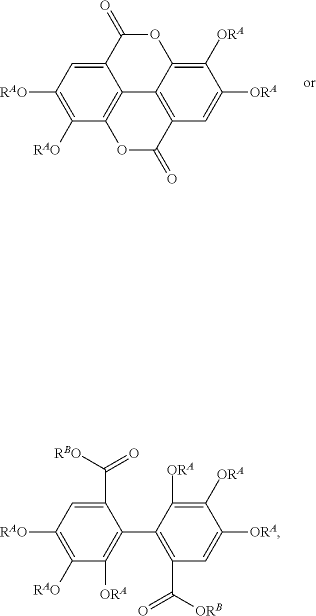

or a salt thereof,

where each R^{A} is independently H, alkyl, acyl, or a group containing a fatty acid; and each R^{B} is independently H, alkyl, or a group containing a fatty acid; provided that at least one R^{A} and/or at least one R^{B}, when present, is a group containing a fatty acid acyl.

An acylated ellagic acid analogue is a compound of the following structure:

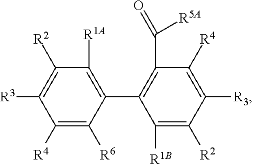

or a salt thereof,
where

each of R², R³, and R⁴ is independently H or —OR^{A};

R⁶ is H or —(CO)—R^{5B};

R^{1A} is H or —OR^{A}, and R^{5A} is —OH or —ORB; or R^{1A} and R^{5A} combine to form —O—;

R^{1B} is H or —OR^{A}, and R^{5B} is absent, —OH, or —ORB; or R^{1B} and R^{5B} combine to form —O—;

each R^{A} is independently H, O-protecting group, alkyl, acyl, or a group containing a fatty acid;

each R^{B} is independently H, O-protecting group, alkyl, or a group containing a fatty acid;

provided that at least one R^{A} and/or at least one R^{B} is a group containing a fatty acid.

Non-limiting examples of ellagic acid analogues include urolithin A, urolithin B, urolithin C, urolithin D, urolithin E, and urolithin M5.

Acylated Stilbenoids

An acylated active agent of the invention may be an acylated stilbenoid. An acylated stilbenoid of the invention may be a stilbenoid, in which one, two, three, four, or five hydroxyl groups are independently replaced with a substituent —OR, where each R is independently selected from the group consisting of an acyl, alkyl, and group including a fatty acid, provided that at least one R is a group including a fatty acid. Stilbenoids are trans-stilbenes that, when not acylated, are substituted with one, two, three, four, or five substituents independently selected from the group consisting of alkoxy (e.g., methoxy) and hydroxyl. Non-limiting examples of stilbenoids include resveratrol, pterostilbene, rhapontigenin, pinostilbene, oxyresveratrol, 4-methoxyresveratrol, and piceatannol. When the stilbenoid is acylated, one or both of the hydroxyl groups in the stilbenoid is independently substituted with a group including a fatty acid acyl or a group including a ketone body or pre-ketone body. In some embodiments, the acylated stilbenoid is an acylated resveratrol.

Acylated Ketone Bodies or Pre-Ketone Bodies

An acylated active agent of the invention may be an acylated ketone body or pre-ketone body. An acylated ketone body is a ketone body or pre-ketone body having one or more hydroxyls substituted with alkyl, acyl, or a group containing a fatty acid. Non-limiting examples or ketone bodies include β-hydroxybutyric acid. Non-limiting examples of pre-ketone bodies include 1,3-butanediol.

Methods

Acylated active agents described herein may be used to treat a cancer in a subject in need thereof.

Without wishing to be bound by theory, the gut microbiome influences metabolism, inflammation and the adaptive immune response which can modulate the progression of cancer and host response to anticancer therapies. Transfer of fecal material from subjects responsive to cancer therapy into germ free mice can render these animals susceptible to cancer therapy. Butyrate, a microbiota metabolite, may inhibit several HDACs as well as act as a ligand for GPR109a which has been implicated in tumor suppression. In vitro, butyrate exerts anti-proliferative and anti-cancer effects in numerous cell lines. Many polyphenols, and their metabolites have also been shown to be supportive of cancer therapy. EGCG may inhibit the proliferation of many tumor types in culture by inhibiting neovascularization promoted by VEGF and other growth factors present in numerous cancer cell lines.

A method of treating cancer in a subject in need thereof may include administering an acylated active agent (e.g., a pharmaceutical or nutraceutical composition containing an acylated active agent) to the subject in need thereof.

In some embodiments, the components of the acylated active agent (e.g., short chain fatty acid acyls (e.g., propionyl, butyryl, or valeryl) and epigallocatechin gallate, myricetin, apigenin, luteolin, resveratrol, or urolithin C) may act synergistically to treat cancer, e.g., upon hydrolysis in the GI tract of the subject receiving the acylated active agent.

Non-limiting examples of cancers include stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, and colorectal cancer.

Additionally or alternatively, acylated active agents described herein may be used for modulating a cancer marker in a subject in need thereof.

A method of modulating a cancer marker in a subject in need thereof may include administering an acylated active agent (e.g., a pharmaceutical or nutraceutical composition containing an acylated active agent) to the subject in need thereof.

In some embodiments, the components of the acylated active agent (e.g., short chain fatty acid acyls (e.g., acetyl) and epigallocatechin gallate) may act synergistically to modulate a cancer marker, e.g., upon hydrolysis in the GI tract of the subject receiving the acylated active agent.

Non-limiting examples of cancer markers include markers for stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, and colorectal cancer. The cancer markers include, for example, a CD4⁺CD25⁺ Treg cell (e.g., CD4⁺CD25⁺Foxp3⁺ Treg cell) count, cytotoxic T cell count, Tₕ1 level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, and 8-iso-prostaglandin F_{2α} (8-iso-PGF2α) level. Modulation (e.g., increase) of a CD4⁺CD25⁺ Treg cell (e.g., CD4⁺CD25⁺Foxp3⁺ Treg cell) count is desirable at the pre-malignancy stages of cancers (e.g., polyposis and pre-malignant stage of cervical cancer).

The cancer markers may be measured using methods known in the art. For example, blood sample analyses may be performed to measure a CD4⁺CD25⁺ Treg cell (e.g., CD4⁺CD25⁺Foxp3⁺ Treg cell) count, cytotoxic T cell count, Tₕ1 level, NFκB level, inducible nitric oxide synthase (iNOS) level, matrix metallopeptidase 9 (MMP9) level, interferon γ (IFNγ) level, interleukin-17 (IL17) level, intercellular adhesion molecule (ICAM) level, CXCL13 level, and 8-iso-prostaglandin F_{2α} (8-iso-PGF2α) level.

In some embodiments, an acylated active agent described herein increases a cancer marker, e.g., CD4⁺CD25⁺ Treg cell (e.g., CD4⁺CD25⁺Foxp3⁺ Treg cell) count, cytotoxic T cell count, Tₕ1 cell count, interferon γ (IFNγ) level, interleukin-17 (IL17) level, or intercellular adhesion molecule (ICAM) level in a subject (e.g., at least 5%, 10%, 15%, 20%, 25%, 30%, 35%, 40%, 45%, 50%, 55%, 60%, 65%, 70%, 75%, 80%, 85%, 90%, 95%, or 98% or more relative to a control group or to the level prior to administration). In certain embodiments, an acylated active agent described herein reduces a cancer marker, e.g., NFκB level, MMP9 level, 8-iso-PGF_{2α} level, or CXCL13 level in a subject (e.g., at least 5%, 10%, 15%, 20%, 25%, 30%, 35%, 40%, 45%, 50%, 55%, 60%, 65%, 70%, 75%, 80%, 85%, 90%, 95%, or 98% or more relative to a control group or to the level prior to administration). In further embodiments, an acylated active agent described herein modulates (increases or decreases) a cancer marker, e.g., Tₕ1 cell count, IgA level, or iNOS level in a subject (e.g., at least 5%, 10%, 15%, 20%, 25%, 30%, 35%, 40%, 45%, 50%, 55%, 60%, 65%, 70%, 75%, 80%, 85%, 90%, 95%, or 98% or more relative to a control group or to the level prior to administration). An attendant doctor or nurse practitioner can determine whether an increase or a decrease in the Tₕ1 cell count, IgA level, or iNOS level is desired.

In particular embodiments, an acylated active agent described herein reduces the viability of tumor cells in in vitro assays or decreases tumor burden in an animal model of cancer. In some embodiments, an acylated active agent described herein reduces pain (e.g., incidence and/or intensity) and/or the need for supportive medication used by a subject, e.g., change in duration of opioid medication or decreases the need for recombinant human granulocyte colony-stimulating factor analogs in a subject (e.g., at least 5%, 10%, 15%, 20%, 25%, 30%, 35%, 40%, 45%, 50%, 55%, 60%, 65%, 70%, 75%, 80%, 85%, 90%, 95%, or 98% or more relative to a control group). In certain embodiments, an acylated active agent described herein reduces the incidence of adverse events in subjects, e.g., change in a subject-reported outcome of CIPN, subject's pain intensity score, percentage of subjects stopping chemotherapy because of sensory peripheral neuropathy, or percentage of subjects requiring a reduction in chemotherapy dose intensity because of adverse events (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to a control group). In particular embodiments, an acylated active agent described herein improves composite outcome measures of disease progression in subjects, e.g., objective response rate, progression free survival, overall survival, response rate in subjects (e.g., at least 5%, 10%, 15%, 20%, 25%, 30%, 35%, 40%, 45%, 50%, 55%, 60%, 65%, 70%, 75%, 80%, 85%, 90%, 95% or 98% or more relative to a control group).

An acylated active agent described herein may be administered alone or in combination with a chemotherapeutic or an anti-cancer immunotherapeutic agent (e.g., a checkpoint inhibitor). For example, such combination therapy may be used for the treatment of renal cancer, melanoma, or non-small cell lung cancer. In a non-limiting example, an acylated active agent disclosed herein may be co-administered to a subject with a PD1/PDL1 inhibitor (e.g., pembrolizumab, nivolumab, avelumab, duvalumab, atezolizumab, AMP-225 (from GlaxoSmithKline), AMP-514 (from GlaxoSmithKline), PDR001 (from Novartis), or BMS-936559 (from Bristol Myers Squibb)), CTLA4 inhibitor (e.g., ipilimumab), or IDO inhibitor (e.g., epacadostat, navoximod, or BMS-986205 (from Bristol Myers Squibb)). Preferably, an acylated active agent disclosed herein may be co-administered to a subject with a PD1/PDL1 inhibitor (e.g., pembrolizumab, nivolumab, avelumab, duvalumab, atezolizumab, AMP-225 (from GlaxoSmithKline), AMP-514 (from GlaxoSmithKline), PDR001 (from Novartis), or BMS-936559 (from Bristol Myers Squibb)) or CTLA4 inhibitor (e.g., ipilimumab).

Pharmaceutical and Nutraceutical Compositions

The acylated active agents disclosed herein may be formulated into pharmaceutical or nutraceutical compositions for administration to human subjects in a biologically compatible form suitable for administration in vivo. Pharmaceutical and nutraceutical compositions typically include an acylated active agent as described herein and a physiologically acceptable excipient (e.g., a pharmaceutically acceptable excipient).

The acylated active agents described herein can also be used in the form of the free acid/base, in the form of salts, zwitterions, or as solvates. All forms are within the scope of the invention. The acylated active agents, salts, zwitterions, solvates, or pharmaceutical or nutraceutical compositions thereof, may be administered to a subject in a variety of forms depending on the selected route of administration, as will be understood by those skilled in the art. The acylated active agents described herein may be administered, for example, by oral, parenteral, buccal, sublingual, nasal, rectal, patch, pump, or transdermal administration, and the pharmaceutical or nutraceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, an acylated active agent disclosed herein can be administered alone or in admixture with a pharmaceutical or nutraceutical carrier selected regarding the intended route of administration and standard pharmaceutical practice. Pharmaceutical and nutraceutical compositions for use in accordance with the present invention thus can be formulated in a conventional manner using one or more physiologically acceptable carriers including excipients and auxiliaries that facilitate processing of acylated active agents disclosed herein into preparations which can be used pharmaceutically.

This disclosure also includes pharmaceutical and nutraceutical compositions which can contain one or more physiologically acceptable carriers. In making the pharmaceutical or nutraceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives. Nutraceutical compositions may be administered enterally (e.g., orally). A nutraceutical composition may be a nutraceutical oral formulation (e.g., a tablet, powder, lozenge, sachet, cachet, elixir, suspension, emulsion, solution, syrup, or soft or hard gelatin capsule), food additive (e.g., a food additive as defined in 21 C.F.R. § 170.3), food product (e.g., food for special dietary use as defined in 21 C.F.R. § 105.3), or dietary supplement (e.g., where the active agent is a dietary ingredient (e.g., as defined in 21 U.S.C. § 321(ff))). Active agents can be used in nutraceutical applications and as food additive or food products. Non-limiting examples of compositions including an active agent of the invention are a bar, drink, shake, powder, additive, gel, or chew.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

These pharmaceutical and nutraceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21^{st} Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical and nutraceutical formulation. In preparing a formulation, the acylated active agents can be milled to provide the appropriate particle size prior to combining with the other ingredients. If the acylated active agent is substantially insoluble, it can be milled to a particle size of less than 200 mesh. If the acylated active agent is substantially water soluble, the particle size can be adjusted by milling to provide a substantially uniform distribution in the formulation, e.g., about 40 mesh.

Dosages

The dosage of the acylated active agent used in the methods described herein, or pharmaceutically acceptable salts or prodrugs thereof, or pharmaceutical or nutraceutical compositions thereof, can vary depending on many factors, e.g., the pharmacodynamic properties of the acylated active agent; the mode of administration; the age, health, and weight of the recipient; the nature and extent of the symptoms; the frequency of the treatment, and the type of concurrent treatment, if any; and the clearance rate of the acylated active agent in the subject to be treated. One of skill in the art can determine the appropriate dosage based on the above factors. The acylated active agents used in the methods described herein may be administered initially in a suitable dosage that may be adjusted as required, depending on the clinical response. In general, a suitable daily dose of an acylated active agent disclosed herein will be that amount of the acylated active agent that is the lowest dose effective to produce a therapeutic effect. Such an effective dose will generally depend upon the factors described above.

An acylated active agent disclosed herein may be administered to the subject in a single dose or in multiple doses. When multiple doses are administered, the doses may be separated from one another by, for example, 1-24 hours, 1-7 days, or 1-4 weeks. The acylated active agent may be administered according to a schedule, or the acylated active agent may be administered without a predetermined schedule. It is to be understood that, for any particular subject, specific dosage regimes should be adjusted over time according to the individual need and the professional judgment of the person administering or supervising the administration of the compositions.

The acylated active agents may be provided in a dosage form. In some embodiments, the unit dosage form may be an oral unit dosage form (e.g., a tablet, capsule, suspension, liquid solution, powder, crystals, lozenge, sachet, cachet, elixir, syrup, and the like) or a food product serving (e.g., the active agents may be included as food additives or dietary ingredients). In certain embodiments, the dosage form is designed for administration of at least one acylated active agent disclosed herein, where the total amount of an administered acylated active agent is from 0.1 g to 10 g (e.g., 0.5 g to 9 g, 0.5 g to 8 g, 0.5 g to 7 g, 0.5 g to 6 g, 0.5 g to 5 g, 0.5 g to 1 g, 0.5 g to 1.5 g, 0.5 g to 2 g, 0.5 g to 2.5 g, 1 g to 1.5 g, 1 g to 2 g, 1 g to 2.5 g, 1.5 g to 2 g, 1.5 g to 2.5 g, or 2 g to 2.5 g). In other embodiments, the acylated active agent is consumed at a rate of 0.1 g to 10 g per day (e.g., 0.5 g to 9 g, 0.5 g to 8 g, 0.5 g to 7 g, 0.5 g to 6 g, 0.5 g to 5 g, 0.5 g to 1 g per day, 0.5 g to 1.5 g per day, 0.5 g to 2 g per day, 0.5 g to 2.5 g per day, 1 g to 1.5 g per day, 1 g to 2 g per day, 1 g to 2.5 g per day, 1.5 g to 2 g per day, 1.5 g to 2.5 g per day, or 2 g to 2.5 g per day) or more. The attending physician ultimately will decide the appropriate amount and dosage regimen, an effective amount of the acylated active agent disclosed herein may be, for example, a total daily dosage of, e.g., between 0.5 g and 5 g (e.g., 0.5 to 2.5 g) of any of the acylated active agent described herein. Alternatively, the dosage amount can be calculated using the body weight of the subject. Preferably, when daily dosages exceed 5 g/day, the dosage of the acylated active agent may be divided across two or three daily administration events.

In the methods of the invention, the time period during which multiple doses of an acylated active agent disclosed herein are administered to a subject can vary. For example, in some embodiments doses of the acylated active agents are administered to a subject over a time period that is 1-7 days; 1-12 weeks; or 1-3 months. In other embodiments, the acylated active agents are administered to the subject over a time period that is, for example, 4-11 months or 1-30 years. In yet other embodiments, the acylated active agents disclosed herein are administered to a subject at the onset of symptoms. In any of these embodiments, the amount of the acylated active agent that is administered may vary during the time period of administration. When an acylated active agent is administered daily, administration may occur, for example, 1, 2, 3, or 4 times per day.

Formulations

An acylated active agent described herein may be administered to a subject with a pharmaceutically acceptable diluent, carrier, or excipient, in unit dosage form. Conventional pharmaceutical practice may be employed to provide suitable formulations or compositions to administer the acylated active agent to subjects suffering from a disorder. Administration may begin before the subject is symptomatic.

Exemplary routes of administration of the acylated active agents disclosed herein or pharmaceutical or nutraceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The acylated active agents desirably are administered with a physiologically acceptable carrier (e.g., a pharmaceutically acceptable carrier). Pharmaceutical formulations of the acylated active agents described herein formulated for treatment of the disorders described herein are also part of the present invention. In some preferred embodiments, the acylated active agents disclosed herein are administered to a subject orally. In other preferred embodiments, the acylated active agents disclosed herein are administered to a subject topically.

Formulations for Oral Administration

The pharmaceutical and nutraceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with physiologically acceptable excipients (e.g., pharmaceutically acceptable excipients). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other physiologically acceptable excipients (e.g., pharmaceutically acceptable excipients) can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of acylated active agents, or by incorporating the acylated active agent into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the acylated active agents and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical and nutraceutical vehicles.

Formulations for Buccal Administration

Dosages for buccal or sublingual administration typically are 0.1 to 500 mg per single dose as required. In practice, the physician determines the actual dosing regimen which is most suitable for an individual subject, and the dosage varies with the age, weight, and response of the particular subject. The above dosages are exemplary of the average case, but individual instances exist where higher or lower dosages are merited, and such are within the scope of this invention.

For buccal administration, the compositions may take the form of tablets, lozenges, etc. formulated in a conventional manner. Liquid drug formulations suitable for use with nebulizers and liquid spray devices and electrohydrodynamic (EHD) aerosol devices will typically include a acylated active agent disclosed herein with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension of acylated active agents disclosed herein. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., U.S. Pat. Nos. 5,112,598 and 5,556,611, each of which is herein incorporated by reference).

Formulations for Nasal or Inhalation Administration

The acylated active agents may also be formulated for nasal administration. Compositions for nasal administration also may conveniently be formulated as aerosols, drops, gels, and powders. The formulations may be provided in a single or multidose form. In the case of a dropper or pipette, dosing may be achieved by the subject administering an appropriate, predetermined volume of the solution or suspension. In the case of a spray, this may be achieved, for example, by means of a metering atomizing spray pump.

The acylated active agents may further be formulated for aerosol administration, particularly to the respiratory tract by inhalation and including intranasal administration. The acylated active agents for nasal or inhalation administration will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the acylated active agent in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form includes an aerosol dispenser, it will contain a propellant, which can be a compressed gas, e.g., compressed air or an organic propellant, e.g., fluorochlorohydrocarbon. The aerosol dosage forms can also take the form of a pump-atomizer.

Formulations for Parenteral Administration

The acylated active agents described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. For example, to prepare such a composition, the acylated active agents disclosed herein may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:
(1) “Drug Injection:” a liquid preparation that is a drug substance (e.g., an acylated active agent disclosed herein or a solution thereof);
(2) “Drug for Injection:” the drug substance (e.g., an acylated active agent disclosed herein) as a dry solid that will be combined with the appropriate sterile vehicle for parenteral administration as a drug injection;
(3) “Drug Injectable Emulsion:” a liquid preparation of the drug substance (e.g., an acylated active agent disclosed herein) that is dissolved or dispersed in a suitable emulsion medium; (4) “Drug Injectable Suspension:” a liquid preparation of the drug substance (e.g., an acylated active agent disclosed herein) suspended in a suitable liquid medium; and
(5) “Drug for Injectable Suspension:” the drug substance (e.g., an acylated active agent disclosed herein) as a dry solid that will be combined with the appropriate sterile vehicle for parenteral administration as a drug injectable suspension.

Exemplary formulations for parenteral administration include solutions of the acylated active agents prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the acylated active agents or biologically active agents within acylated active agents. Other potentially useful parenteral delivery systems for acylated active agents include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the acylated active agent. Exemplary formulations for parenteral release of the acylated active agent include: aqueous solutions, powders for reconstitution, cosolvent solutions, oil/water emulsions, suspensions, oil-based solutions, liposomes, microspheres, and polymeric gels.

Preparation of Acylated Active Agents

Acylated active agents may be prepared using synthetic methods and reaction conditions known in the art. Optimum reaction conditions and reaction times may vary depending on the reactants used. Unless otherwise specified, solvents, temperatures, pressures, and other reaction conditions may be selected by one of ordinary skill in the art.

### Ester Preparation Strategy #1 (Acylation)

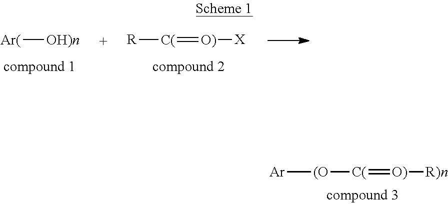

In Scheme 1, a polyphenolic compound, compound 1 where n represents an integer from 1 to 15, is treated with an acylating agent, compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents relative to compound 1.

The product, compound 3, can be purified by methods known to those of skill in the art.

Ester Preparation Strategy #2 (Acylation)

In some cases, the polyphenolic compound 1 may contain a functional group, Y, required to remain unreacted in the course of ester formation. In this case, it is appropriate to protect the functional group, Y, in the polyphenolic compound from acylation. This functional group may be an amino group or a hydroxyl group or other functionality with a labile hydrogen attached to a heteroatom. Such polyphenol esters can be prepared according to Scheme 2.

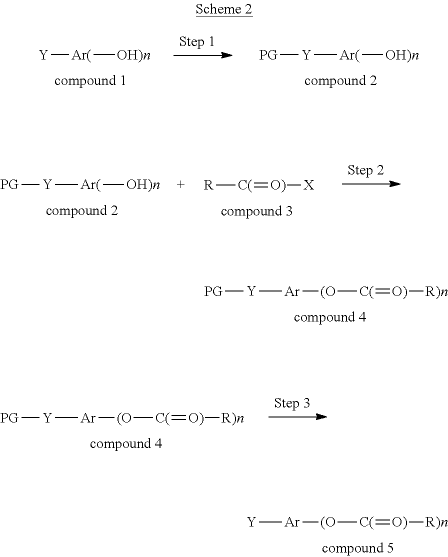

In Scheme 2 Step 1, compound 1, a polyphenolic compound containing a functional group Y with a labile hydrogen in need of protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 2. Compound 2 can be purified by methods known to those of skill in the art.

In Scheme 2 Step 2, compound 2 is treated with an acylating agent, compound 3, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents, relative to compound 3. Compound 4 can be purified by methods known to those of skill in the art.

In Scheme 2 Step 3, compound 4 is subjected to conditions that cleave the protecting group, PG.

In the case of a BOC protecting group, the protecting group of compound 4 is removed under acidic conditions to give compound 5 of the invention. Suitable acids include trifluoroacetic acid, hydrochloric acid, p-toluenesulfonic acid and the like.

In the case of an FMOC protecting group, the protecting group of compound 4 is removed under basic conditions to give compound 5 of the invention. Suitable bases include piperidine, triethylamine and the like. Suitable solvents include DMF, NMP dichoromethane and the like. The FMOC group is also removed under non-basic conditions such as by treatment with tetrabutylammonium fluoride trihydrate in a suitable solvent such as DMF. The FMOC group is also removed by catalytic hydrogenation. Suitable catalysts for hydrogenation include 10% palladium-on-charcoal and palladium (II) acetate and the like. Suitable solvents for hydrogenation include DMF, ethanol, and the like

In the case of a benzyloxycarbonyl or benzyl protecting group the protecting group of compound 4 is removed by hydrogenation to give compound 5. Suitable catalysts for hydrogenation include 10% Palladium-on-charcoal and Palladium acetate and the like. Suitable solvents for hydrogenation include DMF, ethanol, methanol, ethyl acetate, and the like. The product, compound 5, can be purified by methods known to those of skill in the art.

### Ester Preparation Strategy #3 (Acylation)

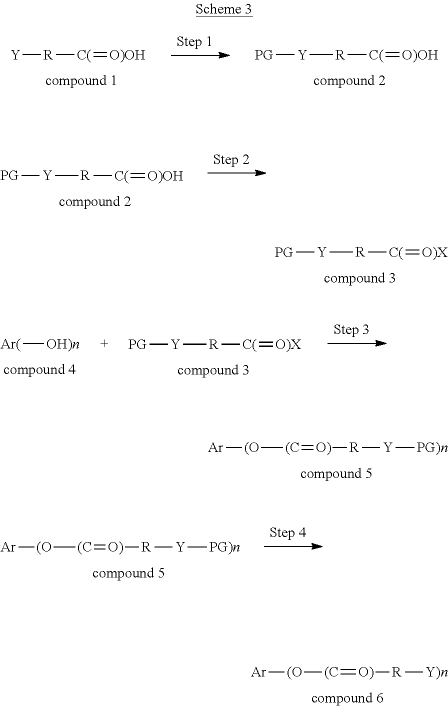

In Scheme 3 Step 1, compound 1, an acyl compound containing a functional group Y with a labile hydrogen in need on protection, is treated with a protecting reagent such as BOC anhydride, benzyoxycarbonyl chloride, FMOC chloride, benzyl bromide and the like in an appropriate solvent, optionally in the presence of a catalyst to provide compound 2 scheme 3. Compound 2 can be purified by methods known to those of skill in the art.

In Scheme 3 Step 2, compound 2 is treated with an activating reagent such as thionyl chloride, phosphorus oxychloride, EDC or EEDQ or the like to generate the activated acyl compound 3.

In Scheme 3 Step 3, the polyphenol compound 4 is treated with the activated acyl compound 3, in an appropriate solvent, optionally in the presence of a catalyst. Suitable catalysts include pyridine, dimethylaminopyridine, trimethylamine and the like to generate compound 5. The catalyst can be used in quantities ranging from 0.01 to 1.1 equivalents, relative to compound 3. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. The activated acyl compound 3 can be used in quantities ranging from 0.5 to 15 equivalents relative to compound 4.

In Scheme 3 Step 4, compound 5 is subjected to conditions designed to cleave the protecting group, PG, illustrated in Scheme 2 above. The product, compound 6, can be purified by methods known to those of skill in the art.

### Ester Preparation Strategy #4 (Acylation)

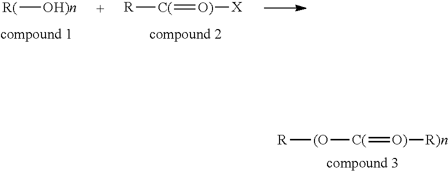

In Scheme 4 Step 1 a poly-ol compound, compound 1, where R represents a non-aromatic cyclic or acyclic moiety and n represents an integer from 1 to 15, is treated with an acylating agent, compound 2, in an appropriate solvent, optionally in the presence of a catalyst. Suitable catalysts include pyridine, dimethylaminopyridine, trimethylamine and the like. The catalyst can be used in quantities ranging from 0.01 to 1.1 equivalents, relative to compound 2. Suitable solvents include methylene chloride, ethyl acetate, diethyl ether, tetrahydrofuran, 1,4-dioxane, 1,2-dimethoxyethane, toluene, combinations thereof and the like. Reaction temperatures range from −10° C. to the boiling point of the solvent used; reaction completion times range from 1 to 96 h. Suitable acylating agents include acyl chlorides, acyl fluorides, acyl bromides, carboxylic acid anhydrides whether symmetrical or not. A suitable acylating agent may also be generated in situ by prior reaction of a carboxylic acid with an activating reagent such as EDC or EEDQ or the like. The acylating agents can be used in quantities ranging from 0.5 to 15 equivalents, relative to compound 1. The product, compound 3, can be purified by methods known to those of skill in the art.

### Ester Preparation Strategy #5 (Baeyer-Villiger Oxidation)

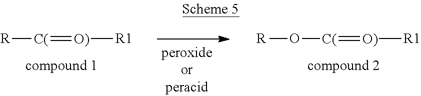

In Scheme 5 Step 1, a ketone compound, compound 1, where R and R1 represent non-aromatic cyclic or acyclic moieties, is treated with a peroxide or peroxyacid agent, such as meta-chloroperbenzoic acid, performic acid, peracetic acid, hydrogen peroxide, tert-butyl hydroperoxide and the like in an appropriate solvent, optionally in the presence of a catalyst. Suitable solvents include methylene chloride, diethyl ether, combinations thereof and the like. Suitable catalysts include BF₃, carboxylic acids, and the like. Reaction temperatures range from −10° C. to the boiling point of the solvent used; reaction completion times range from 1 to 96 h. The product, compound 2, can be purified by methods known to those of skill in the art.

The R and R1 groups of compound 1 in Scheme 5 may optionally include additional ketone functionality that can undergo reaction. In addition the R and R1 groups of compound 1 may form a ring.

### Ester Preparation Strategy #6 (Mitsunobu Reaction)

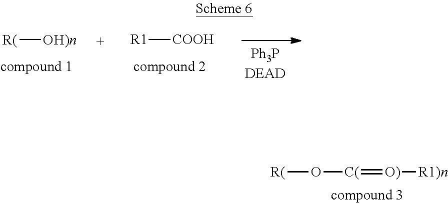

In Scheme 6 Step 1, a mixture of an alcohol compound, compound 1, where R represents a non-aromatic cyclic or acyclic moiety, and a carboxylic acid, compound 2 where R1 represents an alkanoyl group optionally substituted with one or more protected hydroxyl groups or oxo is treated with triphenylphosphine and a diazo compound such as diethylazodicarboxylate (DEAD) and the like in an appropriate solvent. Suitable solvents include methylene chloride, THF, acetonitrile, toluene, diethyl ether, combinations thereof and the like. Reaction temperatures range from −10° C. to the boiling point of the solvent used; reaction completion times range from 1 to 96 h. The product, compound 3 can be purified by methods known to those of skill in the art.

Where compound 3 is optionally substituted by one or more protected alcohol groups deprotection is accomplished by the methods illustrated in Scheme 2 above.

### Ester Preparation Strategy #7 (Nucleophilic Alkylation)

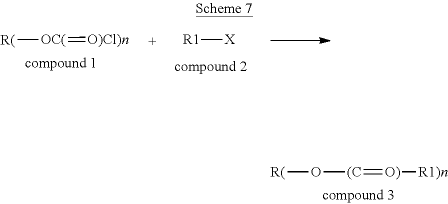

In Scheme 7 Step 1, a chloroformate compound, compound 1, where R represents an aromatic moiety or a non-aromatic cyclic or acyclic moiety, is treated, in an appropriate solvent, with an organometallic compound, compound 2 where R1 represents an alkyl group optionally substituted with one or more protected hydroxyl groups and X represents a metal such as Cu, Zn, Mg which is optionally coordinated by one or more counterions, such as chloride. Suitable solvents include methylene chloride, THF, acetonitrile, toluene, diethyl ether, combinations thereof, and the like. Reaction temperatures range from −10° C. to the boiling point of the solvent used; reaction completion times range from 1 to 96 h. The product, compound 3, can be purified by methods known to those of skill in the art.

Compound 1 can be prepared from the corresponding alcohol or polyol compounds by standard methods familiar to one skilled in the art.

Where compound 2 is optionally substituted by one or more protected alcohol groups deprotection is accomplished by the methods illustrated in Scheme 2 above.

Further modification of the initial product by methods known in the art and illustrated in the examples below, may be used to prepare additional compounds of this invention.

The following examples are meant to illustrate the invention. They are not meant to limit the invention in any way.

### EXAMPLE

### Example 1. Preparation of Exemplary Acylated Active Agents

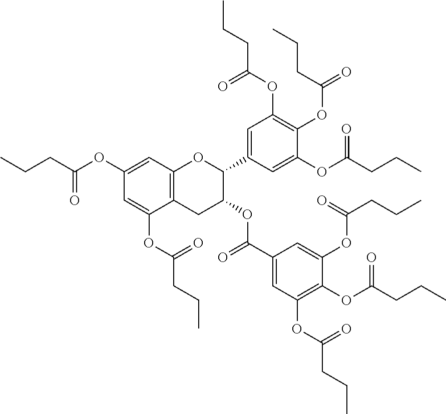 [SMILES: CCCC(=O)Oc1cc(OC(=O)CCC)c2c(c1)O[C@H](c1cc(OC(=O)CCC)c(OC(=O)CCC)c(OC(=O)CCC)c1)[C@H](OC(=O)c1cc(OC(=O)CCC)c(OC(=O)CCC)c(OC(=O)CCC)c1)C2]

### Compound 1: [(2R,3R)-5,7-di(butanoyloxy)-2-[3,4,5-tri(butanoyloxy)phenyl]chroman-3-yl] 3,4,5-tri(butanoyloxy)benzoate

Butyryl chloride (6.03 mL) was added to a stirred solution of epigallocatechin gallate (2.0 g) and pyridine (6.28 mL) in dichloromethane (20 mL) over 2 h between −5° C. to 5° C. The resulting mixture was stirred overnight at room temperature. The reaction mixture was then diluted with dichloromethane (100 mL), washed sequentially with water (50 mL), 2N HCl (50 mL), saturated sodium bicarbonate (50 mL), and brine. The organic layer was evaporated in vacuo, and the resulting crude material was purified by flash chromatography by 30% ethyl acetate/heptane to give compound 1 (800 mg, 18%). ¹H NMR (CDCl₃): 7.6 (s, 2H), 7.22 (s, 2H), 6.78 (s, 1H), 6.6 (s, 1H), 5.62 (t, 1H), 5.18 (s, 1H), 2.98-3.02 (m, 2H), 2.4-2.6 (m, 16H), 1.6-1.8 (m, 16H), 0.92-1.02 (m, 24H).

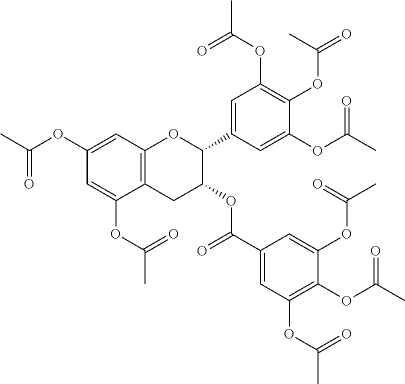 [SMILES: CC(=O)Oc1cc(OC(C)=O)c2c(c1)O[C@H](c1cc(OC(C)=O)c(OC(C)=O)c(OC(C)=O)c1)[C@H](OC(=O)c1cc(OC(C)=O)c(OC(C)=O)c(OC(C)=O)c1)C2]

### Compound 2: [(2R,3R)-5,7-diacetoxy-2-(3,4,5-triacetoxyphenyl)chroman-3-yl] 3,4,5-triacetoxybenzoate

Acetic anhydride (6.1 mL) was added dropwise to epigallocatechin gallate (2.0 g) in pyridine (20 mL) at 0° C., and the resulting mixture was stirred overnight at room temperature. Water was added to the reaction mixture, and the solid was filtered and washed with aq. 1N HCl (10 mL) and heptane (20 mL). The solid was then dissolved in dichloromethane and passed through a silica gel filter column with dichloromethane as a mobile phase to furnish compound 2 (1.0 g, 28%) upon evaporation of volatiles. ¹H NMR (CDCl₃): 7.6 (s, 2H), 7.2 (s, 2H), 6.75 (s, 1H), 6.6 (s, 1H), 5.6 (t, 1H), 5.19 (s, 1H), 2.98-3.02 (m, 2H), 2.18-2.28 (m, 24H).

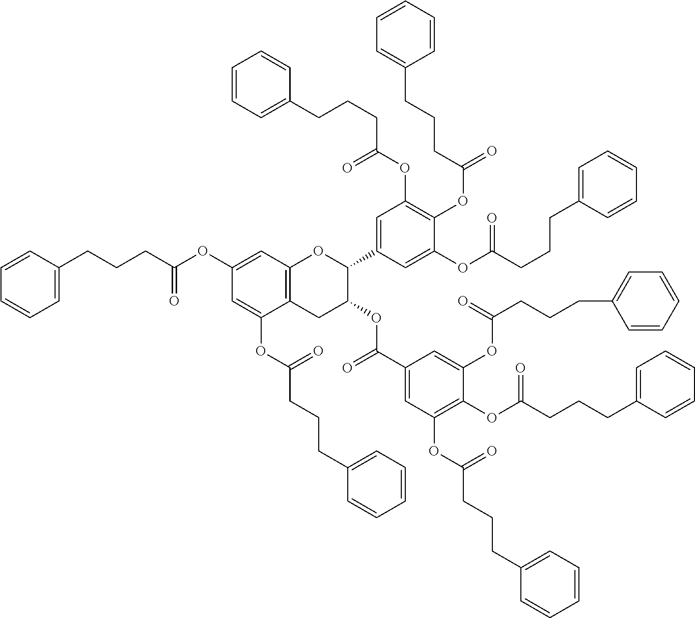 [SMILES: O=C(CCCc1ccccc1)Oc1cc(OC(=O)CCCc2ccccc2)c2c(c1)O[C@H](c1cc(OC(=O)CCCc3ccccc3)c(OC(=O)CCCc3ccccc3)c(OC(=O)CCCc3ccccc3)c1)[C@H](OC(=O)c1cc(OC(=O)CCCc3ccccc3)c(OC(=O)CCCc3ccccc3)c(OC(=O)CCCc3ccccc3)c1)C2]

### Compound 3: [(2R,3R)-5,7-bis(4-phenylbutanoyloxy)-2-[3,4,5-tris(4-phenylbutanoyloxy)phenyl]chroman-3-yl] 3,4,5-tris(4-phenylbutanoyloxy)benzoate

### Step 1

To a solution of 4-phenylbutanoic acid (3 g, 18.27 mmol) and SOCl₂ (10.87 g, 91.35 mmol, 6.63 mL) in dichloromethane (50 mL) is added one drop of DMF, then the mixture stirred at 20° C. for 5 h. The solvent is removed in vacuum and toluene (20 mL) added to the mixture. The mixture is concentrated in vacuo to afford 4-phenylbutanoyl chloride (3.5 g, crude).

### Step 2

To a solution of [(2R,3R)-5,7-dihydroxy-2-(3,4,5-trihydroxyphenyl)chroman-3-yl] 3,4,5-trihydroxybenzoate (1 g, 2.18 mmol) and K₂CO₃ (4.52 g, 32.72 mmol) in acetonitrile (100 mL) was added a solution of 4-phenylbutanoyl chloride (7.97 g, 43.63 mmol) in acetonitrile (10 mL), then the mixture was stirred at 20° C. for 10 h. The mixture was filtered, and the filtrate was concentrated in vacuum. The crude product was purified by silica gel chromatography (petroleum ether/ethyl acetate, 20:1-1:1) to afford compound 3 (2.2 g, 1.28 mmol, 58.7% yield) as a white solid. LC/MS (M+H₃O⁺):1645.1

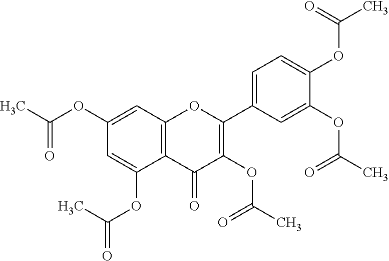 [SMILES: CC(=O)Oc1cc(OC(C)=O)c2c(=O)c(OC(C)=O)c(-c3ccc(OC(C)=O)c(OC(C)=O)c3)oc2c1]

### Compound 4: [2-acetoxy-4-(3,5,7-triacetoxy-4-oxo-chromen-2-yl)phenyl] acetate

To a mixture of 2-(3,4-dihydroxyphenyl)-3,5,7-trihydroxy-chromen-4-one (1 g) and acetic anhydride (2.36 g) in THF (40 mL) was added K₂CO₃ (3.2 g) at 25° C., then the mixture was stirred at 55° C. for 12 h. Additional acetic anhydride was added (3 equiv.) and the mixture and stirred for another 3 h. The reaction mixture was concentrated in vacuum and purified by reverse phase prep-HPLC (C18; water (0.05% HCl)-ACN gradient) to give compound 4 (0.837 g, 49%) as a white solid. LCMS: 513.2 (M+H⁺) ¹H NMR (400 MHz, CDCl₃). 7.742-7.703 (m, 2H), 7.373-7.346 (m, 2H), 6.888 (s, 1H), 2.443, (s, 3H), 2.356 (s, 6H), 2.350 (s, 6H).

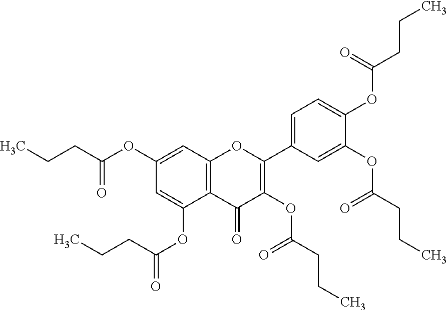 [SMILES: CCCC(=O)Oc1cc(OC(=O)CCC)c2c(=O)c(OC(=O)CCC)c(-c3ccc(OC(=O)CCC)c(OC(=O)CCC)c3)oc2c1]

### Compound 5: [2-butanoyloxy-4-[3,5,7-tri(butanoyloxy)-4-oxo-chromen-2-yl]phenyl] butanoate

To a mixture of 2-(3,4-dihydroxyphenyl)-3,5,7-trihydroxy-chromen-4-one (1 g) and butanoyl chloride (3.53 g) in THF (40 mL) was added TEA (3.35 g) at 25° C., then the mixture was stirred at 55° C. for 12 h. The reaction mixture was concentrated in vacuum and purified by reverse phase prep-HPLC (C18, water (0.05% HCl)-ACN gradient) to give compound 5 (1.13 g, 52% yield) as a colorless solid. LCMS: 653.3 (M+H⁺) ¹H NMR (400 MHz, CDCl₃). 7.666-7.608 (m, 2H), 7.292-7.210 (m, 2H), 6.880 (s, 1H), 2.542 (t, 2H), 2.535-2.484 (m, 8H), 1.753 (m, 10H), 1.020-0.997 (m, 12H), 0.949 (t, 3H).

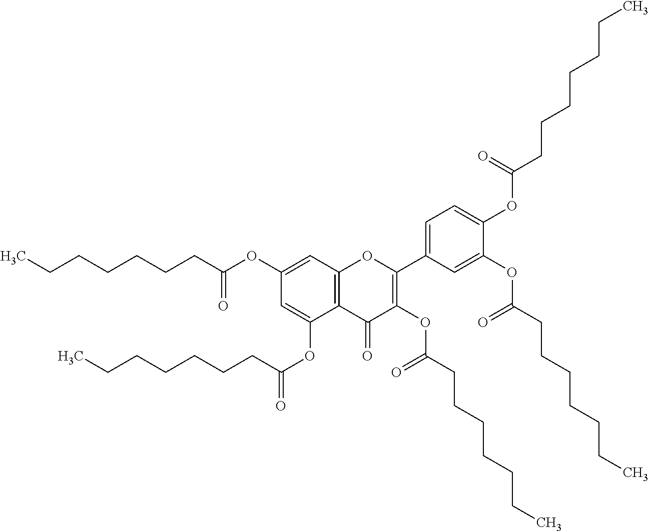 [SMILES: CCCCCCCC(=O)Oc1cc(OC(=O)CCCCCCC)c2c(=O)c(OC(=O)CCCCCCC)c(-c3ccc(OC(=O)CCCCCCC)c(OC(=O)CCCCCCC)c3)oc2c1]

### Compound 6: [2-octanoyloxy-4-[3,5,7-tri(octanoyloxy)-4-oxo-chromen-2-yl] phenyl] octanoate

To a mixture of 2-(3,4-dihydroxyphenyl)-3,5,7-trihydroxy-chromen-4-one (0.32 g) and octanoyl chloride (1.72 g) in THF (20 mL) was added TEA (1.07 g) at 25° C. Then the mixture was stirred at 55° C. for 12 h. A portion of the solvent was removed in vacuum and the precipitate was collected by filtration to give compound 6 (0.20 g, 20%) as a white solid. ¹H NMR (400 MHz, CDCl₃). 7.709-7.655 (m, 2H), 7.329-7.301 (m, 2H), 6.837 (s, 1H), 2.723 (t, 2H), 2.612-2.539 (m, 8H), 1.751 (m, 10H), 1.412-1.309 (m, 40H), 0.896 (m, 15H).

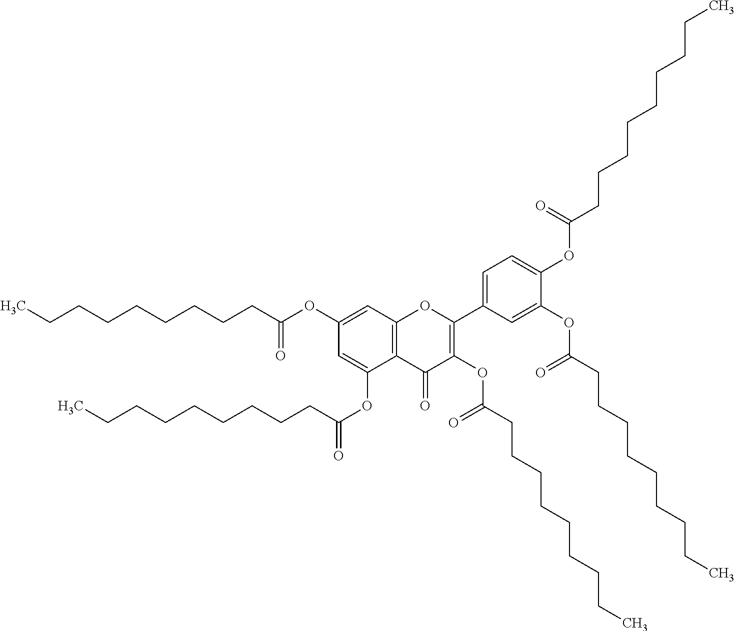 [SMILES: CCCCCCCCCC(=O)Oc1cc(OC(=O)CCCCCCCCC)c2c(=O)c(OC(=O)CCCCCCCCC)c(-c3ccc(OC(=O)CCCCCCCCC)c(OC(=O)CCCCCCCCC)c3)oc2c1]

### Compound 7: [2-decanoyloxy-4-[3,5,7-tris(decanoyloxy)-4-oxo-chromen-2-yl] phenyl] decanoate

To a mixture of 2-(3,4-dihydroxyphenyl)-3,5,7-trihydroxy-chromen-4-one (1 g) and decanoyl chloride (6.31 g) in THF (50 mL) was added TEA (3.35 g) at 25° C., then the mixture was stirred at 55° C. for 12 h. A portion of the solvent was removed in vacuum and the precipitate was collected by filtration to give compound 7 (2.47 g, 69%) as a white solid. ¹H NMR (400 MHz, CDCl₃). 7.772-7.669 (m, 2H), 7.343-7.321 (m, 2H), 6.685 (s, 1H), 2.736 (t, 2H), 2.610-2.551 (m, 8H), 1.762 (m, 10H), 1.557-1.295 (m, 50H), 0.899 (m, 15H).

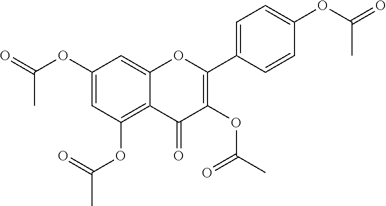 [SMILES: CC(=O)Oc1ccc(-c2oc3cc(OC(C)=O)cc(OC(C)=O)c3c(=O)c2OC(C)=O)cc1]

### Compound 8: [4-(3,5,7-triacetoxy-4-oxo-chromen-2-yl)phenyl] acetate

To a mixture of 3,5,7-trihydroxy-2-(4-hydroxyphenyl)chromen-4-one (2 g) in pyridine (15 mL) was added acetyl acetate (30 g), and then the mixture was stirred at 15° C. for 12 hr under N₂ atmosphere. The solvent was removed under reduced pressure and the residue was poured into crushed ice with vigorous stirring. The solid precipitate was collected by filtration and washed with cold water and then with methanol. Compound 8 (2.1 g, 65% yield) was obtained as a white solid. LCMS: 455.0 (M+H⁺) ¹H NMR (400 MHz, CDCl₃) 7.858 (d, 2H), 7.339 (d, 1H), 7.278-7.257 (m, 2H), 6.883 (d, 1H), 2.447 (s, 3H), 2.357 (s, 6H), 2.333 (s, 3H)

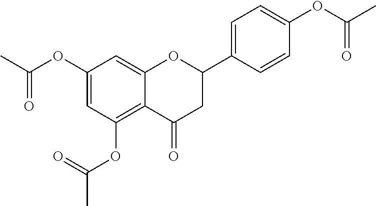 [SMILES: CC(=O)Oc1ccc(C2CC(=O)c3c(OC(C)=O)cc(OC(C)=O)cc3O2)cc1]

### Compound 9: [4-(5,7-diacetoxy-4-oxo-chroman-2-yl)phenyl] acetate

5,7-dihydroxy-2-(4-hydroxyphenyl)chroman-4-one (0.500 g) was dissolved with pyridine (10 mL), and then acetyl acetate (0.844 g) was added. The reaction mixture was stirred at 15° C. for 12 h. The mixture reaction was concentrated under reduced pressure. The residue was purified by column chromatography (SiO₂, petroleum ether/ethyl acetate gradient) to give compound 9 (0.300 g, 39% yield) as a white solid. LCMS: 416.1 (M+H₂O)⁺ ¹H NMR (400 MHz, CDCl₃) 7.468 (d, 2H), 7.166 (d, 2H), 6.793 (d, 1H), 6.551 (d, 1H), 5.497 (dd, 1H), 3.039 (dd, 1H), 2.783 (dd, 1H), 2.393 (s, 3H), 2.326 (s, 3H), 2.308 (s, 3H).

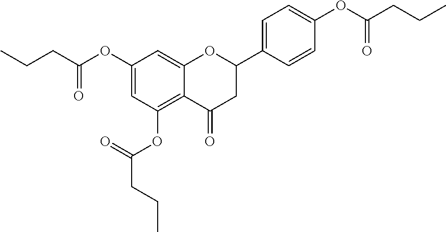 [SMILES: CCCC(=O)Oc1ccc(C2CC(=O)c3c(OC(=O)CCC)cc(OC(=O)CCC)cc3O2)cc1]

### Compound 10: [4-[5,7-di(butanoyloxy)-4-oxo-chroman-2-yl]phenyl] butanoate

To a solution of 5,7-dihydroxy-2-(4-hydroxyphenyl)chroman-4-one (0.500 g) in pyridine (10 mL), was added butanoyl butanoate (1.02 g). The reaction mixture was stirred at 15° C. for 12 h. The mixture was concentrated. The residue was purified by column chromatography (SiO₂, petroleum ether/ethyl acetate gradient) to give compound 10 (0.325 g, 34% yield) as a white solid. LCMS: 500.2 (M+H₂O)⁺ ¹H NMR (400 MHz, CDCl₃) 7.463 (d, 2H), 7.158 (d, 2H), 6.786 (d, 1H), 6.536 (d, 1H), 5.483 (m, 1H), 3.031 (m, 1H), 2.662 (m, 1H), 2.586-2.524 (m, 6H), 1.837-1.785 (m, 6H), 1.089-1.021 (m, 9H)

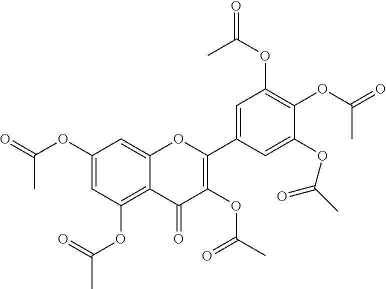 [SMILES: CC(=O)Oc1cc(OC(C)=O)c2c(=O)c(OC(C)=O)c(-c3cc(OC(C)=O)c(OC(C)=O)c(OC(C)=O)c3)oc2c1]

### Compound 11: [3,5-diacetoxy-4-oxo-2-(3,4,5-triacetoxyphenyl)chromen-7-yl] acetate

To a solution of 3,5,7-trihydroxy-2-(3,4,5-trihydroxyphenyl)chromen-4-one (1 g) in pyridine (10 mL) was added acetyl acetate (15.26 g), then the mixture was stirred at 15° C. for 16 h. The solvent was removed and the mixture was poured into ice water under stirring. The solid was filtered, washed with water and dried in vacuum to give compound 11 (1.1 g, 61% yield) as a gray solid. LCMS 571.1 (M+H⁺) ¹H NMR (400 MHz, CDCl₃) 7.260 (s, 2H), 7.349 (d, 1H), 6.886 (d, 1H), 2.441 (s, 3H), 2.372 (s, 3H), 2.353 (s, 3H), 2.341 (s, 3H), 2.333 (s, 6H)

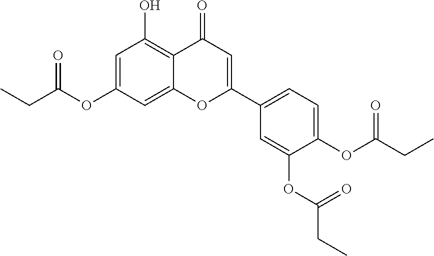 [SMILES: CCC(=O)Oc1cc(O)c2c(=O)cc(-c3ccc(OC(=O)CC)c(OC(=O)CC)c3)oc2c1]

### Compound 12: 4-(5-hydroxy-4-oxo-7-(propionyloxy)-4H-chromen-2-yl)-1,2-phenylene dipropionate

Propionic anhydride (1.33 mL, 10.4 mmol) was added dropwise to a stirred solution of luteolin (0.3 g, 1.04 mmol) in anhydrous pyridine (2.5 mL, 31.2 mmol) at 0° C. under N₂ atmosphere. The stirred solution was allowed to come to room temperature and reaction was monitored to completion by LCMS. The solution was diluted with 30 mL ethyl acetate and washed with H₂O (30 mL), 1M HCl (30 mL), H₂O (30 mL), and saturated NaHCO₃ (30 mL). The organic layer was dried over sodium sulfate, filtered, and concentrated by rotary evaporation. The crude residue was purified by flash chromatography (silica, 10-100% ethyl acetate in hexanes) to yield compound 12 (0.073 g, 15%) as an off-white solid. 1H-NMR (DMSO-d6, 400 MHz): δ 12.75 (s, 1H), 8.07 (m, 2H), 7.5 (m, 1H), 7.15 (s, 1H), 7.12 (d, 1H), 6.66 (d, 1H), 2.59-2.66 (m, 6H), 1.11-1.17 (m, 9H)

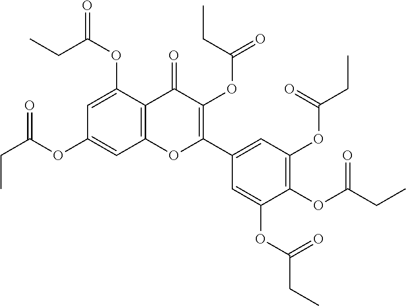 [SMILES: CCC(=O)Oc1cc(OC(=O)CC)c2c(=O)c(OC(=O)CC)c(-c3cc(OC(=O)CC)c(OC(=O)CC)c(OC(=O)CC)c3)oc2c1]

### Compound 13: [4-oxo-3,5-di(propanoyloxy)-2-[3,4,5-tri(propanoyloxy)phenyl]chromen-7-yl] propanoate

Propionic anhydride (2 mL, 15.6 mmol) was added dropwise to a stirred solution of myricetin (0.5 g, 1.56 mmol) in anhydrous pyridine (2.78 mL, 47.1 mmol) at 0° C. under N₂ atmosphere. The stirred solution was allowed to come to room temperature and reaction was monitored to completion by LCMS. The solution was diluted with 30 mL ethyl acetate and washed with H₂O (30 mL), 1M HCl (30 mL), H₂O (30 mL), and saturated NaHCO₃ (30 mL). The organic layer was dried over sodium sulfate, filtered, and concentrated by rotary evaporation. The crude residue was purified by flash chromatography (silica, 10-100% ethyl acetate in hexanes) and fractions were concentrated by rotary evaporation to yield Compound 13 (0.31 g, 30%) as a white solid. ¹H-NMR (DMSO-d₆, 400 MHz): δ 7.77 (s, 2H), 7.64 (d, 1H), 7.16 (d, 1H), 2.60-2.70 (m, 12H), 1.07-1.17 (m, 18H)

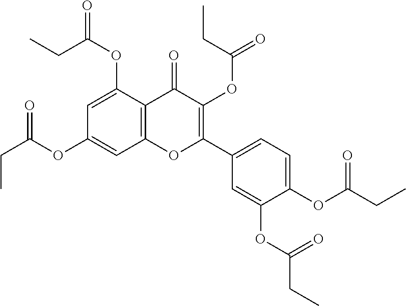 [SMILES: CCC(=O)Oc1cc(OC(=O)CC)c2c(=O)c(OC(=O)CC)c(-c3ccc(OC(=O)CC)c(OC(=O)CC)c3)oc2c1]

### Compound 14: [4-[4-oxo-3,5,7-tri(propanoyloxy)chromen-2-yl]-2-propanoyloxy-phenyl] propanoate

Propionic anhydride (2.1 mL, 16.5 mmol) was added dropwise to a stirred solution of quercetin (0.5 g, 1.65 mmol) in anhydrous pyridine (3.98 mL, 49.5 mmol) at 0° C. under N₂ atmosphere. The resulting stirred solution was allowed to come to room temperature and reaction was monitored to completion by LCMS. The solution was diluted with 30 mL ethyl acetate and washed with H₂O (30 mL), 1M HCl (30 mL), H₂O (30 mL), and saturated NaHCO₃ (30 mL). The organic layer was dried over sodium sulfate, filtered, and concentrated by rotary evaporation. The crude residue was purified by flash chromatography (silica, 10-100% ethyl acetate in hexanes) and fractions were concentrated by rotary evaporation to yield Compound 14 (0.1 g, 10% yield) as a white solid. 1H-NMR (DMSO-d6, 400 MHz): δ 7.85 (m, 2H), 7.66 (d, 1H), 7.54 (d, 1H), 7.18 (d, 1H), 2.62-2.89 (m, 10H), 1.09-1.19 (m, 20H)

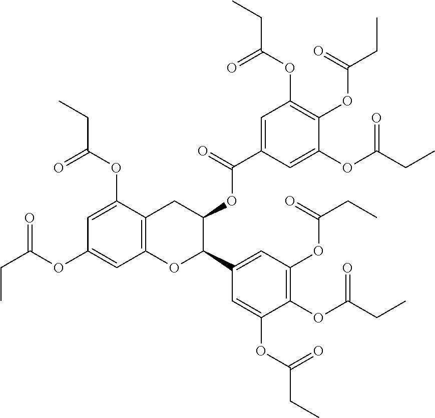 [SMILES: CCC(=O)Oc1cc(OC(=O)CC)c2c(c1)O[C@H](c1cc(OC(=O)CC)c(OC(=O)CC)c(OC(=O)CC)c1)[C@H](OC(=O)c1cc(OC(=O)CC)c(OC(=O)CC)c(OC(=O)CC)c1)C2]

### Compound 15: [(2R,3R)-5,7-di(propanoyloxy)-2-[3,4,5-tri(propanoyloxy)phenyl]chroman-3-yl] 3,4,5-tri(propanoyloxy)benzoate

Propionic anhydride (2.78 mL, 21.8 mmol) was added dropwise to a stirred solution of epigallocatechin gallate (0.5 g, 1.09 mmol) in anhydrous pyridine (2.61 mL, 32.6 mmol) at 0° C. under N₂ atmosphere. The resulting stirred solution was allowed to come to room temperature and reaction was monitored to completion by LCMS. The solution was diluted with 30 mL ethyl acetate and washed with H₂O (30 mL), 1M HCl (30 mL), H₂O (30 mL), and saturated NaHCO₃ (30 mL). The organic layer was dried over sodium sulfate, filtered, and concentrated by rotary evaporation. The crude residue was purified by flash chromatography (silica, 10-100% ethyl acetate in hexanes) to yield Compound 15 (0.695 g, 70%) as a white solid. 1H-NMR (DMSO-d6, 400 MHz): δ 7.54 (s, 2H), 7.38 (s, 2H), 6.79 (m, 1H), 6.66 (m, 1H), 5.66 (m, 1H), 5.54 (s, 1H), 3.13-3.17 (m, 1H), 2.96 (d, 1H), 2.5-2.65 (m, 16H), 1.0-1.2 (m, 24H)

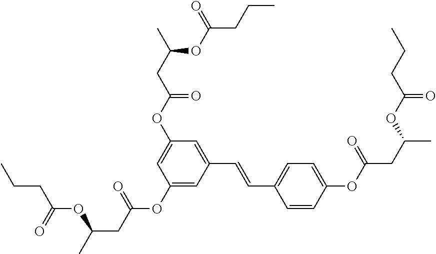 [SMILES: CCCC(=O)O[C@H](C)CC(=O)Oc1ccc(/C=C/c2cc(OC(=O)C[C@@H](C)OC(=O)CCC)cc(OC(=O)C[C@@H](C)OC(=O)CCC)c2)cc1]

### Compound 16: [4-[(E)-2-[3,5-bis[[(3R)-3-butanoyloxybutanoyl]oxy]phenyl]vinyl]phenyl] (3R)-3-butanoyloxy-butanoate

### Step 1: Benzyl (3R)-3-hydroxybutanoate

To a solution of sodium (R)-3-hydroxybutanoate (50 g) in DMF (500 mL) was added dropwise bromomethylbenzene (67.8 g) at 25° C. Then the mixture was stirred at 60° C. for 12 h. Water (800 mL) was added to the reaction mixture, and extracted with EtOAc (550 mL). The organic layer was washed with brine (230 mL) and dried over Na₂SO₄, filtered and concentrated. The residue was purified by flash silica gel chromatography (petroleum ether/ethyl acetate=100/1 to 40/1) to give benzyl (3R)-3-hydroxybutanoate (57 g, 66.6%) as colorless oil which was used directly in the next step.

### Step 2: Benzyl (3R)-3-butanoyloxybutanoate

To a solution of pyridine (55.7 g) in CH₂Cl₂ (570 mL) was added benzyl (3R)-3-hydroxybutanoate (57 g) and 4-dimethylaminopyridine (1.15 g) at 25° C. Butanoyl chloride (43.8 g) was added dropwise to the mixture under N₂ and then stirred at 25° C. for 12 h. The mixture was concentrated, the residue was diluted with EtOAc (300 mL) and the organic layer was washed with H₂O (550 mL), brine (270 mL), dried over Na₂SO₄, filtered and concentrated. The residue was purified by flash silica gel chromatography (petroleum ether/ethyl acetate, 100:1 to 70:1) to give benzyl (3R)-3-butanoyloxybutanoate (54 g, 62.6%) as a colorless oil.

LCMS: 265.1 (M+H⁺)

### Step 3

To a suspension of Pd/C 10% (9 g) in EtOAc (1300 mL) was added benzyl (3R)-3-butanoyloxybutanoate (54 g) at 25° C. The reaction mixture was stirred at 25° C. under H2 (15 Psi) for 4 h. The mixture was filtered and concentrated to give (3R)-3-butanoyloxybutanoic acid (30 g) as colorless oil.

### Step 4: [4-[(E)-2-[3,5-bis[[(3R)-3-butanoyloxybutanoyl]oxy]phenyl]vinyl]phenyl] (3R)-3-butanoyloxy-butanoate

To a solution of 5-[(E)-2-(4-hydroxyphenyl)vinyl]benzene-1,3-diol (0.25 g) and (3R)-3-butanoyloxybutanoic acid (0.76 g) in CH₂Cl₂ (7.5 mL) was added N,N′-dicyclohexylcarbodiimide (0.29 g) in CH₂Cl₂ (5 mL). 4-Dimethylaminopyridine (0.040 g) was added to the mixture at 25° C., and the mixture was stirred for 12 h. The mixture was cooled to 0° C., petroleum ether (10 mL) was added and the mixture was stirred for 15 min, then filtered and concentrated. The residue was dissolved with EtOAc (5 mL), washed with 0.5 N HCl (18 mL) and brine (8 mL), dried over Na₂SO₄, filtered and concentrated. The residue was purified by reverse phase prep-HPLC (C18; water (0.05% HCl)-acetonitrile gradient) to give [4-[(E)-2-[3,5-bis[[(3R)-3-butanoyloxybutanoyl]oxy]phenyl]vinyl]phenyl] (3R)-3-butanoyloxy-butanoate (0.060 g, 7%) as a colorless oil. LCMS: 697.4 (M+H⁺) ¹H NMR (400 MHz, CDCl₃): δ 7.494 (m, 2H), 7.12-7.042 (m, 6H), 6.824 (m, 1H), 5.428, (m, 3H), 2.909-2.785 (m, 6H), 2.303 (m, 6H), 1.696-1.658 (m, 6H), 1.527 (d, 9H), 0.956 (t, 9H) ppm

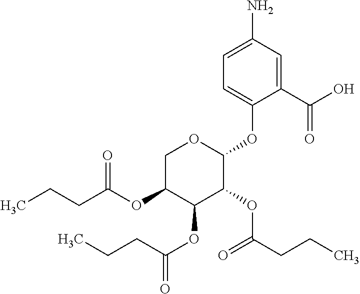 [SMILES: CCCC(=O)O[C@@H]1[C@@H](OC(=O)CCC)[C@@H](Oc2ccc(N)cc2C(=O)O)OC[C@@H]1OC(=O)CCC]

### Compound 17: 5-amino-2-[(2R,3R,4S,5S)-3,4,5-tri(butanoyloxy)tetrahydropyran-2-yl]oxy-benzoic acid

### Step 1

2-Hydroxy-4-nitro-benzoic acid (20 g) and KHCO₃ (13.1 g) were suspended in DMF (100 mL). To the suspension was added benzyl bromide (22.4 g) and the reaction mixture was stirred at room temperature overnight. Water (150 mL) was added and the resulting mixture was extracted with ethyl acetate (250 mL). The organic phase was separated and washed twice with water, brine, and dried over Na₂SO₄. The solvent was removed under reduced pressure and the residue was purified by column chromatography (hexanes/ethyl acetate gradient). Recrystallization from 15% ethyl acetate in hexanes provided benzyl 2-hydroxy-4-nitro-benzoate (10.5 g).

### Step 2

Benzyl 2-hydroxy-4-nitro-benzoate (8.5 g), (3R,4S,5S)-2-hydroxytetrahydro-2H-pyran-3,4,5-triyl tributyrate (7.5 g) and triphenylphosphine (8.2 g) were dissolved in THF (150 mL) and stirred at 0° C. To this mixture was added di-t-butyl azodicarboxylate (7.2 g) and stirring was continued at 0° C. for 1 h, then at room temperature overnight. The reaction mixture was concentrated and purification by column chromatography (hexanes/ethyl acetate gradient) provided benzyl 5-nitro-2-[(2R,3R,4S,5S)-3,4,5-tri(butanoyloxy)tetrahydropyran-2-yl]oxy-benzoate (1.78 g, 14%).

### Step 3

benzyl 5-nitro-2-[(2R,3R,4S,5S)-3,4,5-tri(butanoyloxy)tetrahydropyran-2-yl]oxy-benzoate (0.095 g) was dissolved in methanol (15 mL) and stirred at room temperature. To this mixture was added 10% Pd/C (0.05 g). The suspension was stirred under a hydrogen atmosphere at room temperature overnight. The reaction mixture was filtered through Celite and washed with methanol. The combined filtrate and washing were concentrated. The residue was purified by reverse phase chromatography (C-18, 0.1% trifluoroacetic acid in acetonitrile and 0.1% trifluoroacetic acid in water) to give 5-amino-2-[(2R,3R,4S,5S)-3,4,5-tri(butanoyloxy)tetrahydropyran-2-yl]wry-benzoic acid (0.045 g, 59%). MS 494.2 (M−H) NMR (DMSO d6): δ 7.223 (m, 1H), 7.139 (m, 1H), 6.997 (s, 1H), 7.851 (d, 1H), 5.469 (m, 1H), 5.350 (m, 1H), 5.239 (m, 1H) 4.127 (d, 1H), 3.672 (d, 1H), 2.490-2.369 (M, 6H), 1.596-1.485 (m, 6H), 0.924-0.818 (m, 9H) ppm

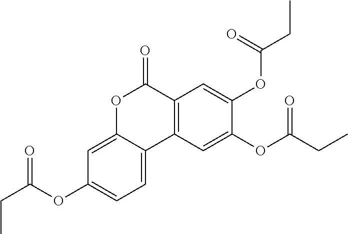 [SMILES: CCC(=O)Oc1ccc2c(c1)oc(=O)c1cc(OC(=O)CC)c(OC(=O)CC)cc12]

### Compound 18: [6-oxo-8,9-di(propanoyloxy)benzo[c]chromen-3-yl] propanoate

Propionic anhydride (2.61 mL, 20.4 mmol) was added dropwise to a stirred solution of urolithin C (0.5 g, 2.04 mmol) in anhydrous pyridine (4.92 mL, 61.2 mmol) at 0° C. under N₂ atmosphere. The stirred solution was allowed to come to room temperature and reaction was monitored to completion by LCMS. The solution was diluted with 30 mL ethyl acetate and washed with H₂O (30 mL), 1M HCl (30 mL), H₂O (30 mL), and saturated NaHCO₃ (30 mL). The organic layer was dried over sodium sulfate, filtered, and concentrated by rotary evaporation. The crude residue was purified by flash chromatography (silica, 10-100% ethyl acetate in hexanes) to yield Compound 18 (0.05 g, 6% yield) as a pink solid. ¹H NMR (DMSO-d6, 400 MHz): δ 8.4 (s, 1H), 8.35 (d, 1H), 8.14 (s, 1H), 7.31 (d, 1H), 7.23 (m, 1H), 2.73-2.63 (m, 6H), 1.21-1.14 (m, 9H) ppm

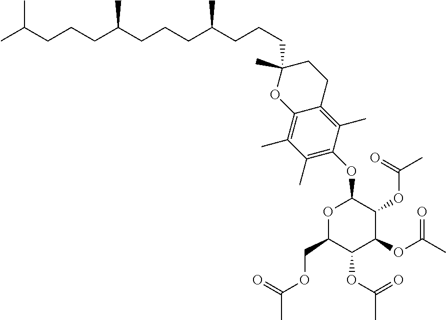 [SMILES: CC(=O)OC[C@H]1O[C@@H](Oc2c(C)c(C)c3c(c2C)CC[C@@](C)(CCC[C@H](C)CCC[C@H](C)CCCC(C)C)O3)[C@H](OC(C)=O)[C@@H](OC(C)=O)[C@@H]1OC(C)=O]

### Compound 19: [(2R,3R,4S,5R,6S)-3,4,5-triacetoxy-6-[(2R)-2,5,7,8-tetramethyl-2-[(4R,8R)-4,8,12-trimethyltridecyl]chroman-6-yl]oxy-tetrahydropyran-2-yl]methyl acetate

### Step 1

3-Bromopyridin-2-ol (5 g) was added to aqueous NaOH (0.34 M, 84.52 mL) and aqueous AgNO₃ (0.68 M, 42.26 mL) at 15° C. The mixture was stirred for 10 min. The reaction mixture was filtered and the solid was washed with H₂O (800 mL) and cooled methanol (200 mL) and dried under reduced pressure to give silver 3-bromopyridin-2-olate (6.5 g, 80.5% yield) as a white solid.

### Step 2

To a solution of (2R,3R,4S,5R,6R)-2-(acetoxymethyl)-6-bromotetrahydro-2H-pyran-3,4,5-triyl triacetate (0.488 g) in toluene (10 mL) was added silver 3-bromopyridin-2-olate (1 g) at 15° C. The mixture was stirred for 3 hr at 120° C. The reaction mixture was filtered and concentrated under reduced pressure and the residue was purified by column chromatography (SiO₂, petroleum ether/ethyl acetate, 1:1) to give (2R,3R,4S,5R,6R)-2-(acetoxymethyl)-6-((3-bromopyridin-2-yl)oxy)tetrahydro-2H-pyran-3,4,5-triyl triacetate (0.500 g, 75% yield) as a white solid.

### Step 3

To a solution of (2R,3R,4S,5R,6R)-2-(acetoxymethyl)-6-((3-bromopyridin-2-yl)oxy)tetrahydro-2H-pyran-3,4,5-triyltriacetate (0.350 g) and α-tocopherol (0.598 g) in CH₂Cl₂ (5 mL) was added BF₃.Et₂O (47%, 0.629 g) at 15° C. The mixture was stirred for 5 hr at 15° C. The reaction mixture was quenched with sodium bicarbonate solution (5 mL), and extracted three times with dichloromethane (10 mL). The combined organic layers were washed with brine (10 mL), dried over Na₂SO₄, filtered and concentrated under reduced pressure. The residue was purified by prep-TLC (SiO₂, petroleum ether/Ethyl acetate, 5:1) to give [(2R,3R,4S,5R,6S)-3,4,5-triacetoxy-6-[(2R)-2,5,7,8-tetramethyl-2-[(4R,8R)-4,8,12-trimethyltridecyl]chroman-6-yl]oxy-tetrahydropyran-2-yl]methyl acetate (0.400 g, 75.7% yield) as a white solid. ¹H NMR (400 MHz, CDCl₃): δ 5.362-5.179 (m, 3H), 4.724 (d, 1H), 4.191-4.049 (m, 3H), 3.536 (m, 1H), 2.568 (m, 2H), 2.152 (s, 3H), 2.120 (s, 3H), 2.105 (s, 3H), 2.082 (s, 3H), 2.054-2.027 (m, 9H), 1.838-1.737 (m, 2H), 1.572-1.042 (m, 24H), 0.882-0.842 (m, 12H) ppm

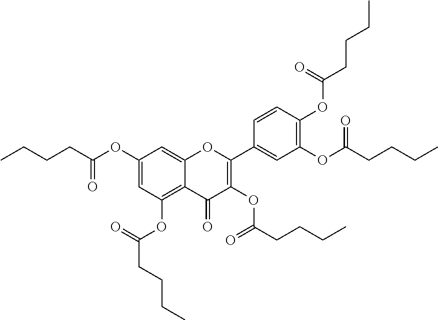 [SMILES: CCCCC(=O)Oc1cc(OC(=O)CCCC)c2c(=O)c(OC(=O)CCCC)c(-c3ccc(OC(=O)CCCC)c(OC(=O)CCCC)c3)oc2c1]

### Compound 20: 2-(3,4-bis(pentanoyloxy)phenyl)-4-oxo-4H-chromene-3,5,7-triyltripentanoate

The above compound can be synthesized in the same manner as Compound 14 using valeric anhydride.

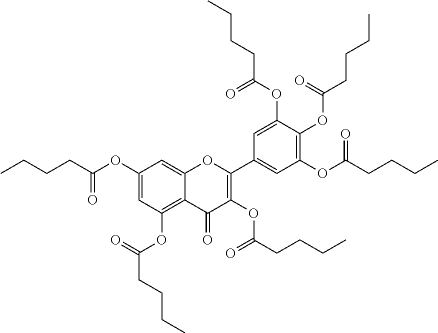 [SMILES: CCCCC(=O)Oc1cc(OC(=O)CCCC)c2c(=O)c(OC(=O)CCCC)c(-c3cc(OC(=O)CCCC)c(OC(=O)CCCC)c(OC(=O)CCCC)c3)oc2c1]

### Compound 21: 4-oxo-2-(3,4,5-tris(pentanoyloxy)phenyl)-4H-chromene-3,5,7-triyltripentanoate

The above compound can be synthesized in the same manner as Compound 14 using valeric anhydride.

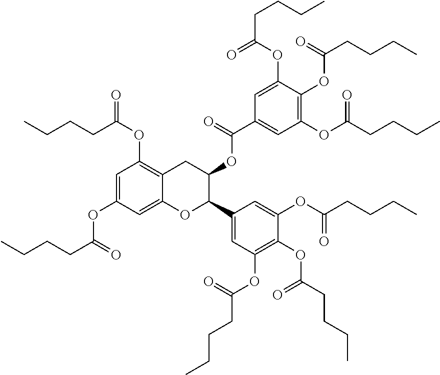 [SMILES: CCCCC(=O)Oc1cc(OC(=O)CCCC)c2c(c1)O[C@H](c1cc(OC(=O)CCCC)c(OC(=O)CCCC)c(OC(=O)CCCC)c1)[C@H](OC(=O)c1cc(OC(=O)CCCC)c(OC(=O)CCCC)c(OC(=O)CCCC)c1)C2]

### Compound 22: 5-((((2R,3R)-5,7-bis(pentanoyloxy)-2-(3,4,5-tris(pentanoyloxy)phenyl)chroman-3-yl)oxy)carbonyl)benzene-1,2,3-triyltripentanoate

The above compound can be synthesized in the same manner as Compound 15 using valeric anhydride.

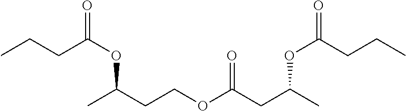 [SMILES: CCCC(=O)O[C@H](C)CCOC(=O)C[C@@H](C)OC(=O)CCC]

### Compound 23: (R)-3-(butyryloxy)butyl (R)-3-(butyryloxy)butanoate

To a solution of [(3R)-3-hydroxybutyl] (3R)-3-hydroxybutanoate (0.400 g), K₂CO₃ (0.784 g) in acetonitrile (5 mL) was added butanoyl chloride (0.532 g), and the mixture was stirred at 15° C. for 12 h. The reaction mixture was concentrated and the residue was purified by column chromatography (SiO₂, petroleum ether/ethyl acetate gradient) to give [(3R)-3-butanoyloxybutyl] (3R)-3-butanoyloxybutanoate (0.220 g, 27.5% yield) as a colorless oil. ¹H NMR (400 MHz, CDCl₃): δ 5.197 (m, 1H), 4.965 (m, 1H), 4.045 (m, 2H), 2.528 (m, 1H), 2.449 (m, 1H), 2.222-2.158 (m, 4H), 1.799 (m, 2H), 1.602-1.546 (m, 4H), 1.222 (d, 3H), 1.182 (d, 3H), 0.884 (t, 3H), 0.874 (t, 3H) ppm

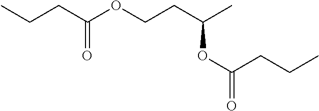 [SMILES: CCCC(=O)OCC[C@@H](C)OC(=O)CCC]

### Compound 24: (R)-butane-1,3-diyl dibutyrate

To a solution of (3R)-butane-1,3-diol (6 g) and K₂CO₃ (23.92 g) in acetonitrile (50 mL) was added butanoyl chloride (18.44 g) and the mixture was stirred at 15° C. for 12 h. The mixture reaction was concentrated. The residue was purified by column chromatography (SiO₂, petroleum ether/ethyl acetate gradient) to give [(3R)-3-butanoyloxybutyl] butanoate (11 g, 64.57% yield) as a colorless oil. LCMS: 248.1 (M+H₃O⁺) ¹H NMR (400 MHz, CDCl₃): δ 5.022 (m, 1H), 4.101 (m, 2H), 2.300-2.247 (m, 4H), 1.885 (m, 2H), 1.679-1.594 (m, 4H), 1.260 (d, 3H), 0.949 (t, 6H) ppm

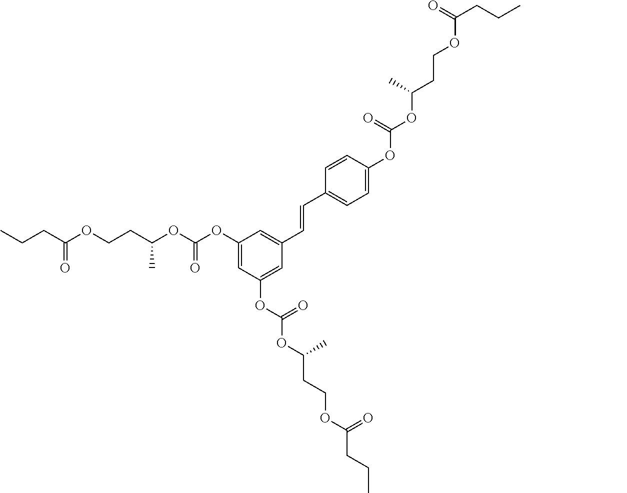 [SMILES: CCCC(=O)OCC[C@@H](C)OC(=O)Oc1ccc(/C=C/c2cc(OC(=O)O[C@H](C)CCOC(=O)CCC)cc(OC(=O)O[C@H](C)CCOC(=O)CCC)c2)cc1]

### Compound 25: [(3R)-3-[4-[(E)-2-[3,5-bis[[(1R)-3-butanoyloxy-1-methyl-propoxy]carbonyloxy]phenyl]vinyl]phenoxy]carbonyloxybutyl] butanoate

To a solution of triphosgene (0.185 g, 0.62 mmol) in THF (10 mL) was added a solution of [(3R)-3-hydroxybutyl] butanoate (0.200 g, 1.25 mmol and TEA (0.189 g, 1.87 mmol) in THF (5 mL) at 0° C. The reaction mixture was stirred for 1 h at 0° C. The mixture reaction was filtered and used directly to next step.

To a solution of 5-[(E)-2-(4-hydroxyphenyl)vinyl]benzene-1,3-diol (0.050 g, 0.219 mmol) and TEA (0.111 g, 1.10 mmol) in THF (5 mL) was added the solution above of [(3R)-3-chlorocarbonyloxybutyl] butanoate in THF at 0° C. The mixture was stirred at 20° C. for 2 h. The reaction mixture was filtered and concentrated and the residue was purified by prep-TLC (SiO₂, petroleum ether/ethyl acetate=5/1) to give [(3R)-3-[4-[(E)-2-[3,5-bis[[(1R)-3-butanoyloxy-1-methyl-propoxy]carbonyloxy]phenyl]vinyl]phenoxy]carbonyloxybutyl] butanoate (0.080 mg, 45% yield) as a colorless oil. LCMS: (M+H₂O⁺): 804.4 ¹H NMR (400 MHz, CDCl₃): δ 7.517 (m, 2H), 7.270-7.018 (m, 7H), 5.103 (m, 3H), 4.260-4.203 (m, 6H), 2.313 (m, 6H), 2.054 (m, 6H), 1.698-1.643 (m, 6H), 1.431 (d, 9H), 0.957 (t, 9H)

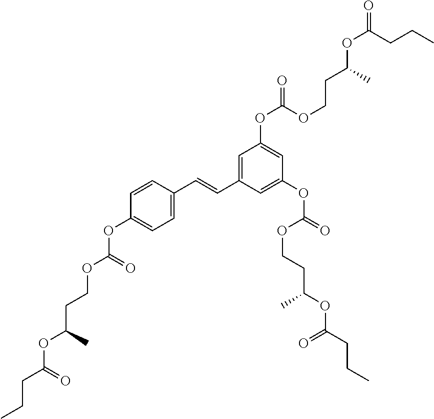 [SMILES: CCCC(=O)O[C@H](C)CCOC(=O)Oc1ccc(/C=C/c2cc(OC(=O)OCC[C@@H](C)OC(=O)CCC)cc(OC(=O)OCC[C@@H](C)OC(=O)CCC)c2)cc1]

### Compound 26: [(1R)-3-[4-[(E)-2-[3,5-bis[[(3R)-3-butanoyloxybutoxy]carbonyloxy]phenyl]vinyl]phenoxy]carbonyloxy-1-methyl-propyl] butanoate

### Step 1

To a solution of triphosgene (0.185 g, 0.624 mmol) in THF (10 mL) was added a solution of [(1R)-3-hydroxy-1-methyl-propyl] butanoate (0.200 g, 1.25 mmol) and TEA (0.189 g, 1.87 mmol) in THF (5 mL) at 0° C. The reaction was stirred for 1 h at 0° C. TLC showed the starting reactant was consumed. The reaction mixture was filtered and concentrated and used directly to next step.

### Step 2

To a solution of 5-[(E)-2-(4-hydroxyphenyl)vinyl]benzene-1,3-diol (0.050 g, 0.219 mmol) and TEA (0.111 g, 1.10 mmol) in THF (5 mL) was added the solution above of [(1R)-3-chlorocarbonyloxy-1-methyl-propyl] butanoate in THF at 0° C. The reaction was stirred at 20° C. for 2 h, then filtered and concentrated. The residue was purified by prep-TLC to give [(1R)-3-[4-[(E)-2-[3,5-bis[[(3R)-3-butanoyloxybutoxy]carbonyloxy]phenyl]vinyl]phenoxy]carbonyloxy-1-methyl-propyl] butanoate (0.084 g, 41% yield) as colorless oil. LCMS: (M+H₂O⁺): 804.3 ¹H NMR (400 MHz, CDCl₃): δ 7.511 (m, 2H), 7.270-6.970 (m, 7H), 5.167-5.087 (m, 3H), 4.360-4.307 (m, 6H), 2.299 (t, 6H), 2.012 (m, 6H), 1.703-1.647 (m, 6H), 1.301 (d, 9H), 0.965 (t, 9H)

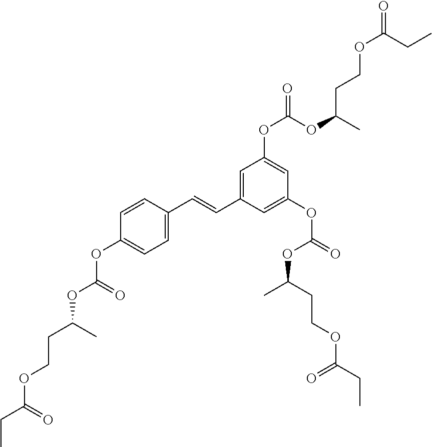 [SMILES: CCC(=O)OCC[C@@H](C)OC(=O)Oc1ccc(/C=C/c2cc(OC(=O)O[C@H](C)CCOC(=O)CC)cc(OC(=O)O[C@H](C)CCOC(=O)CC)c2)cc1]

### Compound 27: [(3R)-3-[4-[(E)-2-[3,5-bis[[(1R)-1-methyl-3-propanoyloxy-propoxy]carbonyloxy]phenyl]vinyl]phenoxy]carbonyloxybutyl] propanoate

To a solution of (3R)-butane-1,3-diol (2 g, 22.2 mmol) and TEA (2.47 g, 24.4 mmol) in DCM (10 mL) was added propanoyl propanoate (3.18 g, 24.4 mmol) and the mixture was stirred at 25° C. for 12 h. The mixture was concentrated and the residue was purified by column chromatography (SiO₂, petroleum ether/ethyl acetate=10/1 to 5:1) to give [(3R)-3-hydroxybutyl] propanoate (1.8 g, 55% yield) as a colorless oil.

To a solution of triphosgene (0.203 g, 0.68 mmol) in THF (10 mL) was added a solution of [(3R)-3-hydroxybutyl] propanoate (0.20 g, 1.37 mmol) and TEA (0.21 g, 2.1 mmol) in THF (5 mL) at 0° C. The mixture was stirred for 1 h at 0° C., then filtered and used directly to next step.

To a solution of 5-[(E)-2-(4-hydroxyphenyl)vinyl]benzene-1,3-diol (0.050 g, 0.219 mmol) and TEA (0.111 g, 1.10 mmol) in THF (5 mL) was added the solution above of [(3R)-3-chlorocarbonyloxybutyl] propanoate in THF at 0° C. The reaction mixture was stirred at 20° C. for 2 h, then filtered and concentrated. The residue was purified by prep-TLC to give [(3R)-3-[4-[(E)-2-[3,5-bis[[(1R)-1-methyl-3-propanoyloxy-propoxy]carbonyloxy]phenyl]vinyl]phenoxy]carbonyloxybutyl] propanoate (0.060 g, 35% yield) as a colorless oil. LCMS: (M+Na⁺): 767.3 ¹H NMR (400 MHz, CDCl₃): δ 7.518 (d, 2H), 7.270-6.976 (m, 7H), 5.032-4.968 (m, 3H), 4.267-4.189 (m, 6H), 2.384-2.328 (m, 6H), 2.066-1.980 (m, 6H), 1.430 (d, 9H), 1.555 (t, 9H)

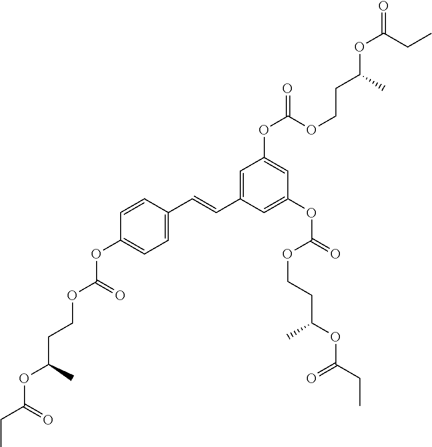 [SMILES: CCC(=O)O[C@H](C)CCOC(=O)Oc1ccc(/C=C/c2cc(OC(=O)OCC[C@@H](C)OC(=O)CC)cc(OC(=O)OCC[C@@H](C)OC(=O)CC)c2)cc1]

### Compound 28: (2R,2′R)-((((5-(E)-4-((((R)-3-(propionyloxy)butoxy)carbonyl)oxy)styryl)-1,3-phenylene)bis(oxy))bis(carbonyl))bis(oxy))bis(butane-4,2-diyl) dipropionate

### Step 1

To a solution of pyridine (1.05 g, 13.3 mmol) in DCM (10 mL) was added (2R)-4-benzyloxybutan-2-ol (1 g, 5.6 mmol) and DMAP (0.022 g, 0.18 mmol) at 0° C. Then propanoyl chloride (0.719 g, 7.77 mmol) was added to the mixture at 0° C. and the mixture was stirred at 25° C. for 3 h under N₂. The reaction mixture was concentrated under reduced pressure and the residue was purified by flash silica gel chromatography (petroleum ether/ethyl acetate=100/1 to 70/1) to give [(1R)-3-benzyloxy-1-methyl-propyl] propanoate (1.2 g, 82% yield) as a colorless oil.

### Step 2

To a solution of 10% Pd/C (0.4 g) in THF (200 mL) was added [(1R)-3-benzyloxy-1-methyl-propyl] propanoate (1.2 g, 5.1 mmol), and the mixture was degassed 3 times and purged with Hz, then stirred at 40° C. for 12 h under Hz, 15 Psi. The mixture reaction was filtered and concentrated to give [(1R)-3-hydroxy-1-methyl-propyl] propanoate (0.70 g) as a colorless oil.

### Step 3

To a solution of triphosgene (0.203 g, 0.684 mmol) in THF (10 mL) was added a solution of [(1R)-3-hydroxy-1-methyl-propyl] propanoate (0.200 g, 1.37 mmol) and TEA (0.208 g, 2.05 mmol) in THF (5 mL) at 0° C. The reaction was stirred for 1 h at 0° C. The reaction mixture was filtered and concentrated and used in next step directly.

### Step 4

To a solution of 5-[(E)-2-(4-hydroxyphenyl)vinyl]benzene-1,3-diol (0.050 g, 0.219 mmol) and TEA (0.111 g, 1.10 mmol) in THF (5 mL) was added the above solution of [(1R)-3-chlorocarbonyloxy-1-methyl-propyl] propanoate in THF at 0° C. The reaction was stirred at 20° C. for 2 h. The mixture reaction was filtered and concentrated and the residue was purified by prep-TLC to give [(1R)-3-[4-[(E)-2-[3,5-bis[[(3R)-3-propanoyloxybutoxy]carbonyloxy]phenyl]vinyl]phenoxy]carbonyloxy-1-methyl-propyl] propanoate (0.030 g, 14.7% yield) as a colorless oil. LCMS: (M+Na⁺): 767.3 ¹H NMR (400 MHz, CDCl₃): δ 7.506 (d, 2H), 7.232-6.997 (m, 7H), 5.149-5.070 (m, 3H), 4.363-4.290 (m, 6H), 2.368-2.312 (m, 6H), 2.062-1.993 (m, 6H), 1.308 (d, 9H), 1.555 (t, 9H)

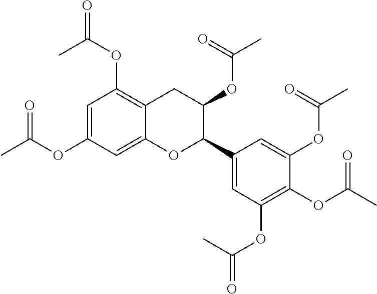 [SMILES: CC(=O)Oc1cc(OC(C)=O)c2c(c1)O[C@H](c1cc(OC(C)=O)c(OC(C)=O)c(OC(C)=O)c1)[C@H](OC(C)=O)C2]

### Compound 29: 5-((2R,3R)-3,5,7-triacetoxychroman-2-yl)benzene-1,2,3-triyltriacetate

The above compound can be synthesized in the same manner as Compound 15 using acetic anhydride.

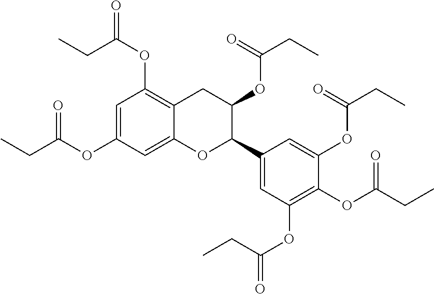 [SMILES: CCC(=O)Oc1cc(OC(=O)CC)c2c(c1)O[C@H](c1cc(OC(=O)CC)c(OC(=O)CC)c(OC(=O)CC)c1)[C@H](OC(=O)CC)C2]

### Compound 30: 5-((2R,3R)-3,5,7-tris(propionyloxy)chroman-2-yl)benzene-1,2,3-triyltripropionate

The above compound can be synthesized in the same manner as Compound 15 using propionic anhydride.

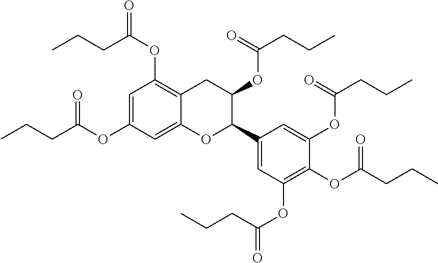 [SMILES: CCCC(=O)Oc1cc(OC(=O)CCC)c2c(c1)O[C@H](c1cc(OC(=O)CCC)c(OC(=O)CCC)c(OC(=O)CCC)c1)[C@H](OC(=O)CCC)C2]

### Compound 31: 5-((2R,3R)-3,5,7-tris(butyryloxy)chroman-2-yl)benzene-1,2,3-triyl tributyrate

The above compound can be synthesized in the same manner as Compound 15 using butyric anhydride.

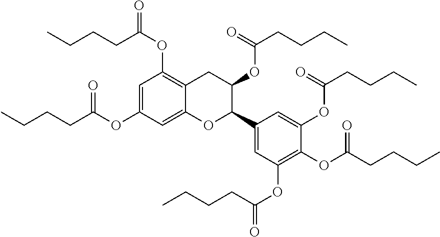 [SMILES: CCCCC(=O)Oc1cc(OC(=O)CCCC)c2c(c1)O[C@H](c1cc(OC(=O)CCCC)c(OC(=O)CCCC)c(OC(=O)CCCC)c1)[C@H](OC(=O)CCCC)C2]

### Compound 32: 5-((2R,3R)-3,5,7-tris(pentanoyloxy)chroman-2-yl)benzene-1,2,3-triyl tripentanoate

The above compound can be synthesized in the same manner as Compound 15 using valeric anhydride.

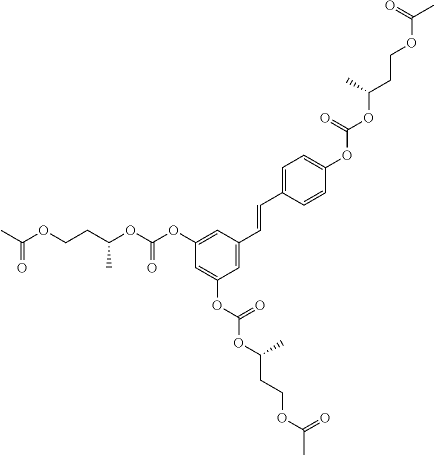 [SMILES: CC(=O)OCC[C@@H](C)OC(=O)Oc1ccc(/C=C/c2cc(OC(=O)O[C@H](C)CCOC(C)=O)cc(OC(=O)O[C@H](C)CCOC(C)=O)c2)cc1]

### Compound 33: [(3R)-3-[4-[(E)-2-[3,5-bis[[(1R)-3-acetoxy-1-methyl-propoxy]carbonyloxy]phenyl]vinyl]phenoxy]carbonyloxybutyl] acetate

### Step 1

To a solution of (3R)-butane-1,3-diol (2.4 g) in pyridine (20 mL) was added Ac₂O (2.17 g) and the mixture was stirred at 15° C. for 12 h. The mixture reaction was concentrated. The residue was purified by column chromatography (SiO₂, petroleum ether/ethyl acetate gradient) to give [(3R)-3-hydroxybutyl] acetate (1.4 g, 35.8% yield) as a colorless oil.

### Step 2

To a solution of triphosgene (0.269 g) in THF (5 mL) was added a solution of [(3R)-3-hydroxybutyl] acetate (0.300 g) and TEA (0.230 g) in THF (5 mL) at 0° C. and the mixture was stirred for 1 h at 15° C. A ˜0.23 M solution of [(3R)-3-chlorocarbonyloxybutyl] acetate (15 mL) was obtained which was used in the next step directly.

### Step 3

To a solution of 5-[(E)-2-(4-hydroxyphenyl)vinyl]benzene-1,3-diol (0.090 g) and TEA (0.218 g) in THF (3 mL) was added a solution of [(3R)-3-chlorocarbonyloxybutyl] acetate (0.23 M, 10 mL) in THF. The reaction mixture was stirred for 5 h at 15° C. The mixture reaction was filtered and concentrated. The residue was purified by prep-TLC (SiO₂, petroleum ether/ethyl acetate, 4:1) to give compound 33 (0.085 g, 28.8% yield as a colorless oil. LCMS: 725.1 (M+Na⁺) ¹H NMR (400 MHz, CDCl₃) 7.520 (d, 2H), 7.244-7.192 (m, 4H), 7.114 (d, 1H), 7.036-6.979 (m, 2H), 5.019 (m, 3H), 4.226 (m, 6H), 2.099-1.995 (m, 6H), 2.055 (s, 6H), 1.442-1.422 (m, 9H).

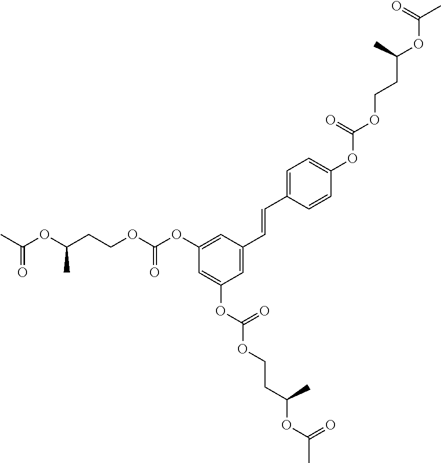 [SMILES: CC(=O)O[C@H](C)CCOC(=O)Oc1ccc(/C=C/c2cc(OC(=O)OCC[C@@H](C)OC(C)=O)cc(OC(=O)OCC[C@@H](C)OC(C)=O)c2)cc1]

### Compound 34: [(1R)-3-[4-[(E)-2-[3,5-bis[[(3R)-3-acetoxybutoxy]carbonyloxy]phenyl]vinyl]phenoxy]carbonyloxy-1-methyl-propyl] acetate

### Step 1

To a solution of NaH (2.35 g, 60%) in THF (100 mL) was added (3R)-3-[tert-butyl (dimethyl)silyl]oxybutan-1-ol (10 g) at 0° C. The mixture was stirred at 15° C. for 1.5 h. Benzyl bromide (10.04 g) was added and the mixture was stirred at 15° C. for 16 h. The reaction mixture was filtered and concentrated under reduced pressure and the residue was purified by column chromatography (SiO₂, petroleum ether) to give [(1R)-3-benzyloxy-1-methyl-propoxy]-tert-butyl-dimethyl-silane (11 g, 55% yield) as a colorless oil.

### Step 2

To a solution of [(1R)-3-benzyloxy-1-methyl-propoxy]-tert-butyl-dimethyl-silane (10 g) in THF (100 mL) was added pyridine hydrofluoride (8.41 g) at 15° C. The mixture was stirred for 2 h at 50° C. The reaction mixture was combined with another batch and concentrated under reduced pressure. The residue was diluted with H₂O (50 mL) and extracted four times with ethyl acetate (50 mL). The combined organic phase was washed with brine (50 mL), dried over Na₂SO4, filtered and concentrated under reduced pressure. The residue was purified by column chromatography (SiO₂, petroleum ether/ethyl acetate gradient) to give (2R)-4-benzyloxybutan-2-ol (5.54 g) as a colorless oil.

### Step 3

To a solution of (2R)-4-benzyloxybutan-2-ol (5.54 g) in pyridine (50 mL) was added Ac₂O (4.71 g) at 15° C. The mixture was stirred for 12 h at 15° C. The reaction mixture was concentrated under reduced pressure and the residue was purified by column chromatography (SiO₂, petroleum ether/ethyl acetate gradient) to give [(1R)-3-benzyloxy-1-methyl-propyl] acetate (4.7 g, 57% yield) as a colorless oil.

### Step 4

To a solution of [(1R)-3-benzyloxy-1-methyl-propyl] acetate (2 g) in THF (20 mL) was added 10% Pd/C (0.027 g). The mixture was stirred under H2 (30 psi) for 16 h at 30° C. The reaction mixture was filtered and concentrated under reduced pressure and the residue was purified by column chromatography (SiO₂, petroleum ether/ethyl acetate gradient) to give [(1R)-3-hydroxy-1-methyl-propyl] acetate (1.07 g, 65% yield) as a colorless oil.

### Step 5

To a solution of [(1R)-3-hydroxy-1-methyl-propyl] acetate (0.300 g) in THF (5 mL) was added a solution of triphosgene (0.337 g) and TEA (0.230 g) in THF (5 mL) at 0° C. The mixture was stirred for 1 h at 15° C. The mixture reaction was filtered and used to next step directly.

### Step 6

To a solution of 5-[(E)-2-(4-hydroxyphenyl)vinyl]benzene-1,3-diol (0.080 g) and TEA (0.194 g) in THF (3 mL) was added a solution of [(1R)-3-chlorocarbonyloxy-1-methyl-propyl] acetate (0.2 M, 10 mL) in THF. The reaction mixture was stirred for 5 h at 15° C. The mixture reaction was filtered and concentrated. The residue was purified by prep-TLC (SiO₂, petroleum ether/ethyl acetate, 4/1) to give compound 34 (0.056 g, 21% yield) as a colorless oil. LCMS: 703.1 (M+H⁺) ¹H NMR (400 MHz, CDCl₃) 7.513 (d, 2H), 7.232-7.181 (m, 4H), 7.154 (d, 1H), 7.129-7.002 (m, 2H), 5.106 (m, 3H), 4.340 (m, 6H), 2.072 (s, 9H), 2.078-1.995 (m, 6H), 1.323-1.282 (m, 9H)

### Example 2. In Vitro Assays

Acylated active agents disclosed herein may be stable under a range of physiological pH levels and cleaved selectively at a desired site of action (for example, in the GI tract, e.g., in the stomach, small intestine, or large intestine) by enzymes present in the local microenvironment. Acylated active agents are tested for chemical stability at a range of pH levels as well as their ability to be degraded in representative in vitro systems. Data for select acylated active agents are shown below.

Assay 1.

Stability of acylated active agents in Simulated Gastric Fluid (SGF). This assay was used to assess the stability of an acylated active agent in a stomach.

Medium was prepared by dissolving 2 g of sodium chloride in 0.6 L in ultrapure water (MilliQ®, Millipore Sigma, Darmstadt, Germany). The pH was adjusted to 1.6 with 1N hydrochloric acid, and the volume was then adjusted to 1 L with purified water.

60 mg FaSSIF powder (Biorelevant™, London, UK) were dissolved in 500 mL buffer (above). Pepsin was added (0.1 mg/mL) (Millipore Sigma, Darmstadt, Germany), and the solution was stirred. The resulting SGF media were used fresh for each experiment.

Test compounds were dissolved in DMSO stock to 1 mM. An aliquot of the DMSO stock solution was removed and diluted in the SGF Media in 15 mL falcon tubes to generate a total compound concentration of 1 μM. A 1 mL aliquot was immediately removed and diluted once with 1 volume of acetonitrile for T0 timepoint. The mixture was sealed and mixed at 37° C. in an incubator. Aliquots (1 mL) were removed at regular intervals and immediately quenched by the addition of 1 volume of acetonitrile. The resulting samples were analyzed by LC/MS to determine degradation rates

Assay 2.

SIF Stability of acylated active agents in Simulated Intestinal Fluid (SIF). This assay was used to assess the stability of an acylated active agent in a small intestine.

Phosphate buffer was prepared by dissolving 0.42 g of sodium hydroxide pellets and 3.95 g of monobasic sodium phosphate monohydrate and 6.19 g of sodium chloride in ultrapure water (MilliQ®, Millipore Sigma, Darmstadt, Germany). The pH was adjusted to 6.7 using aq. HCl and aq. NaOH, as necessary, and the solution was diluted with ultrapure water to produce 1 L of the pH 6.7 buffer.

112 mg FaSSIF powder (Biorelevant™, London, UK) was dissolved in 50 mL of the pH 6.7 buffer. 2 to 3 mL of the resulting solution were then added to 500 mg pancreatin (Millipore Sigma, Darmstadt, Germany). The resulting mixture was agitated by finger tapping the vessel containing the mixture until milky suspension formed. At this time, the remainder of the 50 mL FaSSiF/pH 6.7 buffer solution was added. The resulting suspension was flipped upside down 10 times to produce SIF, which was used fresh.

Test compounds were dissolved in DMSO stock to 1 mM. An aliquot of the DMSO stock solution was removed and diluted in the SIF media in 15 mL falcon tubes to produce a mixture with a tested compound concentration of 1 μM. A 1 mL aliquot was immediately removed and diluted once with 1 volume of acetonitrile for T0 timepoint. The mixture was sealed and agitated at 37° C. in an incubator. Aliquots (1 mL) were removed at regular intervals and immediately quenched by the addition of 1 volume of acetonitrile. The resulting samples were analyzed by LC/MS to determine degradation rates.

Assay 3.

Fecal Incubation Stability. This assay was used to assess the stability of an acylated active agent in a large intestine. All experiments were performed in an anaerobic chamber containing 90% nitrogen, 5% hydrogen and 5% carbon dioxide. Fecal matter in a slurry (15% VW is added to 96 well plates containing YCFA media or other suitable media (1.6 mL). Compounds were added to each individual well to reach a final analyte concentration of 1 or 10 μM, and the material was mixed by pipetting. At set time points a sample was removed, quenched with acetonitrile, and analyzed by LC/MS.

Buffer Assay.

Stability of acylated active agents in a buffer. This assay provides for the assessment of the stability of an acylated active agent at different physiological pH levels.

Compounds are diluted in DMSO, and added in the appropriate quantity to phosphate buffer (pH levels 2, 4, 6, and 8) to reach a total sample concentration of 2 μM. Compounds are incubated at RT, and aliquots are removed at time points 0, 60, 120, 360 and 1440 minutes and analyzed for purity by LC/MS/MS.

TABLE 1

| | Assay 1 (SGF) | Assay 2 (SIF) | Assay 3 |
|---|---|---|---|
| | (% Remaining | (% @ Remaining | (% Remaining |
| Compound | @ 1 hours) | 4 hours) | at 24 h) |
| 1 | C | C | C |
| 2 | C | A | A |
| 3 | | | |
| 4 | C | A | |
| 5 | C | B | |
| 6 | | | |
| 7 | | | |
| 8 | C | | |
| 9 | C | A | |
| 10 | B | A | |
| 11 | C | | |
| 12 | C | A | |
| 13 | C | A | |
| 14 | C | C | C |
| 15 | C | | |
| 16 | C | | A |
| 17 | C | A | C |
| 18 | C | A | |
| 19 | C | C | C |
| 20 | | | |
| 21 | | | |
| 22 | | | |
| 23 | C | | |
| 24 | | | |
| 25 | C | A | |
| 26 | C | B | |
| 27 | C | A | |
| 28 | B | A | |
| 29 | | | |
| 30 | | | |
| 31 | | | |
| 32 | | | |
| 33 | C | A | |
| 34 | C | A | |

In Table 1,
A: <25% of the tested compound remaining;
B: 25-75% of the tested compound remaining; and
C: >75% of the tested compound remaining.

Table 1 shows that, for example, compounds 2, 4, 5, 9, 12, 13, 17, 18, 25, 27, 33, 34 can be selectively delivered to the upper intestine.

### Example 3. In Vivo Evaluation of an Acylated Catechin Polyphenol

Acylated catechin polyphenols disclosed herein may be useful in modulating cancer markers and for treating cancer. This example demonstrates the capability of an exemplary acylated active agent, compound 2, to induce CD4⁺CD25⁺ Treg cells in a subject.

C57BL/6 mice were divided into seven cohorts, as listed in Table 2.

TABLE 2

| | | # of | | | |
|---|---|---|---|---|---|
| Model | Treatment* | animals | Dose** | Frequency | Route |
| HFD-fed | ND | 10 | | Ad libitum | Diet |
| C57BL/6 | HFD | 10 | | Ad libitum | Diet |
| mice | HFD + Acetate | 10 | 5% | Ad libitum | Diet |
| | HFD + EGCG | 10 | 1% | Ad libitum | Diet |
| | HFD + Acetate + | 10 | 5% + 1% | Ad libitum | Diet |
| | EGCG | | | | |
| | Compound 2 | 10 | 6% | Ad libitum | Diet |
| | (EGCG-8A) | | | | |
| | HFD + rosiglitazone | 10 | 0.45 mg/g | Ad libitum | Diet |

*In Table 2, ND means normal diet, HFD means high-fat diet, and EGCG means epigallocatechin gallate.
**In Table 2, dose percentages refer to weight percentage relative to the high fat diet.

The results of this study are illustrated in the FIG., which shows a synergistic induction of CD4⁺CD25⁺ Treg cells by Compound 2 in animals fed a high-fat diet, as compared to the administration of EGCG, acetate, or their combination as separate compounds.

### Example 4. Small Molecules Inhibit the Proliferation of Cancer Cells

Release products of acylated active agents may be cytostatic/cytotoxic to cancer cells of various tissue types and lineages—for example, bladder, blood, breast, colorectal, lung, prostate, skin, or stomach cells. Here such fatty acids, ketones, and catechins are tested for cytotoxicity towards cancer cells. Data for select molecules are shown below.

The cells were prepared as follows. Cells were harvested during logarithmic growth, counted, and adjusted to 4.44×10⁴ cells/mL with culture media. 90 μL of suspended cells were added to wells of 96-well plates for a final cell count of 4×10³ cells/well. The plates were incubated overnight at 37 C with 5% CO₂ overnight.

The test compounds were assayed as follows. Compound solutions were mixed at 10× final concentrations in cell media: 1 μM-10 mM (fatty acids) or 100 nM-1 mM (all other compounds). 104 of solution was added to each well of cells in triplicate for each concentration. Well plates were incubated for 72 h at 37 C with 5% CO₂ and then assayed with CellTiter-Glo by adding 50 μL of CTG solution to each well. Cells were lysed by shaking on an orbital shaker for 5 min. Plates were incubated at room temperature for 20 min and then each well's luminescence was measured. The surviving percentage of cells was determined as:

Survival %=(Lum_{Test article}−Lum_{Medium control})/(Lum_{None treated}−Lum_{Medium control})λ100

Assay 1.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in T24 bladder cancer cells. Efficacy was assessed at nine concentration levels for each compound and the 50% inhibition concentration (IC50) determined for each compound. The data are shown in Tables 3.1 and 3.2.

TABLE 3.1

| | Cell survival at given concentration | | | | | | | | | |
|---|---|---|---|---|---|---|---|---|---|---|
| | 101 | 318 | 1 | 3 | 10 | 32 | 100 | 316 | 1000 | |
| | nM | nM | μM | μM | μM | μM | μM | μM | μM | 1050 (uM) |
| resveratrol | 1.00 | 1.02 | 1.00 | 1.01 | 0.92 | 0.01 | 0.00 | 0.00 | 0.00 | 15.12 |
| EGCG | 1.00 | 0.97 | 1.02 | 1.01 | 0.96 | 0.58 | 0.15 | 0.03 | 0.03 | 37.82 |
| urolithin C | 1.00 | 0.94 | 0.96 | 0.96 | 0.93 | 0.37 | 0.12 | 0.08 | 0.07 | 26.14 |
| L-arabinose | 1.00 | 1.03 | 1.00 | 1.03 | 1.02 | 1.05 | 1.01 | 1.01 | 1.04 | NA |
| EGC | 0.99 | 1.03 | 1.03 | 1.01 | 1.00 | 0.84 | 0.00 | 0.00 | 0.00 | 40.04 |
| myricetin | 1.01 | 0.98 | 1.06 | 1.02 | 0.96 | 0.09 | 0.00 | 0.00 | 0.00 | 19.12 |
| alpha-tocopherol | 1.02 | 1.01 | 0.96 | 1.00 | 0.99 | 1.06 | 0.98 | 1.07 | 1.02 | NA |
| luteolin | 0.99 | 1.02 | 1.03 | 0.94 | 0.85 | 0.35 | 0.09 | 0.08 | 0.06 | 22.76 |
| apigenin | 0.98 | 1.00 | 1.02 | 1.05 | 0.98 | 0.79 | 0.60 | 0.42 | 0.32 | 161.42 |

TABLE 3.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 0.97 | 0.97 | 1.04 | 0.97 | 1.00 | 1.01 | 0.99 | 0.97 | 0.91 | NA |
| butyrate | 0.98 | 1.00 | 1.05 | 0.99 | 0.97 | 0.90 | 0.84 | 0.60 | 0.27 | 4717.41 |
| beta- | 0.97 | 1.00 | 0.98 | 0.95 | 0.96 | 1.02 | 0.99 | 0.99 | 1.00 | NA |
| hydroxybutyrate | | | | | | | | | | |
| butanediol | 1.02 | 1.03 | 0.99 | 1.00 | 1.01 | 1.03 | 1.02 | 1.00 | 1.00 | NA |
| propionate | 0.99 | 0.97 | 0.97 | 0.99 | 0.97 | 0.98 | 0.89 | 0.83 | 0.60 | >10000 |
| valerate | 1.00 | 1.01 | 1.01 | 0.98 | 0.98 | 0.92 | 0.89 | 0.79 | 0.60 | >10000 |

Assay 2.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in Daudi lymphoma cancer cells (a non-Hodgkin lymphoma). Activity was assessed at nine concentration levels for each compound and the 50% inhibition concentration (IC50) determined for each compound. The data are shown in Tables 4.1 and 4.2.

TABLE 4.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 1.00 | 1.01 | 1.02 | 1.02 | 1.01 | 0.14 | 0.00 | 0.00 | 0.00 | 23.88 |
| EGCG | 1.03 | 1.02 | 1.01 | 0.97 | 0.76 | 0.31 | 0.17 | 0.00 | 0.00 | 20.39 |
| urolithin C | 0.99 | 0.99 | 1.00 | 1.00 | 0.89 | 0.36 | 0.18 | 0.16 | 0.07 | 24.09 |
| L-arabinose | 1.00 | 1.00 | 1.00 | 0.99 | 1.02 | 1.02 | 1.03 | 1.02 | 1.02 | NA |
| EGC | 1.00 | 1.01 | 1.01 | 1.03 | 1.02 | 0.99 | 0.00 | 0.00 | 0.00 | 47.50 |
| myricetin | 1.04 | 1.03 | 1.04 | 1.04 | 1.05 | 0.25 | 0.00 | 0.00 | 0.00 | 28.07 |
| alpha- | 0.98 | 0.99 | 1.01 | 1.00 | 1.02 | 1.04 | 1.04 | 1.03 | 1.02 | NA |
| tocopherol | | | | | | | | | | |
| luteolin | 1.00 | 1.02 | 1.03 | 1.06 | 0.85 | 0.33 | 0.10 | 0.06 | 0.16 | 21.62 |
| apigenin | 0.97 | 0.96 | 1.01 | 1.01 | 1.03 | 0.86 | 0.60 | 0.47 | 0.44 | 185.78 |

TABLE 4.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.00 | 0.99 | 0.99 | 1.01 | 1.02 | 1.02 | 1.01 | 0.97 | 0.75 | NA |
| butyrate | 1.01 | 0.99 | 1.00 | 1.00 | 0.99 | 0.87 | 0.46 | 0.19 | 0.13 | 912.15 |
| beta- | 1.01 | 0.99 | 0.98 | 0.98 | 1.02 | 0.99 | 0.99 | 0.96 | 0.91 | NA |
| hydroxybutyrate | | | | | | | | | | |
| butanediol | 0.98 | 0.97 | 0.97 | 0.98 | 1.00 | 1.01 | 1.00 | 1.00 | 1.00 | NA |
| propionate | 0.97 | 0.97 | 0.99 | 0.99 | 1.01 | 0.97 | 0.75 | 0.47 | 0.18 | 2728.85 |
| valerate | 0.99 | 0.98 | 0.98 | 1.01 | 0.95 | 0.91 | 0.76 | 0.39 | 0.12 | 2299.39 |

Assay 3.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in MCF7 breast cancer cells. Cytotoxicity was assessed at nine concentration levels for each compound and the 50% inhibition concentration (IC50) determined for each compound. The data are shown in Tables 5.1 and 5.2.

TABLE 5.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 0.94 | 0.96 | 0.99 | 0.99 | 0.95 | 0.96 | 0.85 | 0.27 | 0.00 | 211.55 |
| EGCG | 1.01 | 0.99 | 0.99 | 0.97 | 0.96 | 0.77 | 0.55 | 0.51 | 0.11 | 231.35 |
| urolithin C | 0.97 | 0.99 | 0.97 | 1.02 | 1.07 | 0.76 | 0.22 | 0.21 | 0.20 | 34.69 |
| L-arabinose | 0.99 | 1.01 | 0.98 | 1.01 | 0.99 | 1.01 | 0.99 | 0.98 | 0.98 | NA |
| EGC | 0.93 | 0.97 | 0.97 | 1.02 | 0.92 | 1.00 | 0.90 | 0.56 | 0.02 | 354.85 |
| myricetin | 1.05 | 0.95 | 1.04 | 1.00 | 0.99 | 1.03 | 0.94 | 0.99 | 0.87 | >1000 |
| alpha- | 1.02 | 1.02 | 1.04 | 1.05 | 1.05 | 1.04 | 1.03 | 1.02 | 1.01 | NA |
| tocopherol | | | | | | | | | | |
| luteolin | 0.96 | 0.99 | 1.03 | 1.04 | 1.08 | 0.60 | 0.31 | 0.26 | 0.24 | 35.96 |
| apigenin | 0.98 | 0.99 | 0.98 | 1.00 | 1.02 | 0.89 | 0.82 | 0.76 | 0.73 | NA |

TABLE 5.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.01 | 1.02 | 0.98 | 1.03 | 1.02 | 1.04 | 0.99 | 0.99 | 0.91 | NA |
| butyrate | 1.01 | 1.03 | 1.02 | 1.00 | 1.04 | 0.98 | 0.82 | 0.54 | 0.24 | 3589.43 |
| beta- | 1.00 | 1.00 | 1.00 | 1.01 | 1.03 | 1.01 | 0.97 | 0.92 | 0.91 | NA |
| hydroxybutyrate | | | | | | | | | | |
| butanediol | 1.00 | 0.99 | 0.98 | 1.02 | 1.03 | 1.01 | 1.00 | 0.98 | 0.96 | NA |
| propionate | 0.99 | 0.97 | 1.03 | 1.02 | 1.02 | 0.94 | 0.91 | 0.77 | 0.51 | >10000 |
| valerate | 1.00 | 1.02 | 1.01 | 0.97 | 1.00 | 0.96 | 0.94 | 0.76 | 0.50 | >10000 |

Assay 4.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in HCT 116 colorectal cancer cells. Cytotoxicity was assessed at nine concentration levels for each compound and the 50% inhibition concentration (IC50) determined for each compound. The data are shown in Tables 6.1 and 6.2.

TABLE 6.1

| | Cell survival at given concentration | | | | | | | | | |
|---|---|---|---|---|---|---|---|---|---|---|
| | 101 | 318 | 1 | 3 | 10 | 32 | 100 | 316 | 1000 | |
| | nM | nM | μM | μM | μM | μM | μM | μM | μM | IC50 (μM) |
| resveratrol | 1.03 | 0.98 | 0.98 | 1.01 | 0.98 | 0.65 | 0.00 | 0.00 | 0.00 | 37.38 |
| EGCG | 1.05 | 0.98 | 1.00 | 1.03 | 1.00 | 0.61 | 0.25 | 0.06 | 0.00 | 46.32 |
| urolithin C | 1.00 | 0.99 | 1.00 | 0.99 | 0.94 | 0.34 | 0.02 | 0.01 | 0.01 | 25.53 |
| L-arabinose | 1.01 | 1.01 | 1.01 | 1.03 | 1.03 | 1.04 | 1.04 | 1.02 | 0.99 | NA |
| EGC | 0.99 | 0.98 | 1.00 | 1.01 | 0.99 | 0.97 | 0.01 | 0.00 | 0.00 | 50.34 |
| myricetin | 1.01 | 1.04 | 1.07 | 1.05 | 1.02 | 0.31 | 0.00 | 0.00 | 0.00 | 26.47 |
| alpha- | 0.99 | 0.98 | 1.00 | 1.01 | 1.01 | 1.03 | 1.03 | 0.98 | 0.97 | NA |
| tocopherol | | | | | | | | | | |
| luteolin | 1.00 | 0.98 | 1.01 | 0.97 | 0.93 | 0.29 | 0.02 | 0.06 | 0.01 | 23.78 |
| apigenin | 0.99 | 1.00 | 1.00 | 0.99 | 0.98 | 0.60 | 0.32 | 0.19 | 0.15 | 45.83 |

TABLE 6.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.01 | 1.03 | 1.03 | 1.03 | 1.06 | 1.02 | 1.02 | 1.00 | 0.84 | >10000 |
| butyrate | 0.98 | 0.99 | 1.02 | 1.01 | 0.97 | 0.92 | 0.66 | 0.18 | 0.01 | 1440.49 |
| beta- | 0.98 | 1.00 | 0.99 | 0.98 | 1.00 | 1.03 | 1.01 | 0.96 | 0.90 | NA |
| hydroxybutyrate | | | | | | | | | | |
| butanediol | 0.98 | 0.96 | 0.98 | 0.97 | 1.00 | 1.02 | 1.01 | 0.99 | 0.95 | NA |
| propionate | 0.98 | 1.02 | 1.03 | 1.03 | 1.06 | 1.03 | 0.98 | 0.66 | 0.19 | 4377.98 |
| valerate | 0.99 | 0.99 | 1.04 | 1.00 | 1.01 | 0.99 | 0.90 | 0.60 | 0.17 | 4066.10 |

Assay 5.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in A549 lung cancer cells. Cytotoxicity was assessed at nine concentration levels for each compound and the 50% inhibition concentration (IC50) determined for each compound. The data are shown in Tables 7.1 and 7.2.

TABLE 7.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 0.98 | 0.99 | 0.98 | 0.98 | 0.98 | 0.96 | 0.88 | 0.50 | 0.00 | 316.63 |
| EGCG | 1.02 | 1.05 | 1.03 | 1.04 | 0.99 | 0.77 | 0.33 | 0.21 | 0.01 | 67.43 |
| urolithin C | 0.99 | 0.99 | 1.02 | 1.04 | 1.04 | 1.02 | 0.36 | 0.16 | 0.12 | 93.50 |
| L-arabinose | 1.01 | 1.03 | 1.04 | 1.02 | 1.03 | 1.03 | 1.02 | 1.02 | 1.04 | NA |
| EGC | 0.98 | 1.00 | 1.00 | 1.00 | 0.94 | 0.96 | 0.94 | 0.81 | 0.09 | 639.68 |
| myricetin | 1.00 | 1.00 | 0.99 | 1.04 | 1.01 | 0.99 | 0.99 | 0.98 | 0.79 | >1000 |
| alpha- | 1.03 | 1.05 | 1.06 | 1.04 | 1.08 | 1.09 | 1.08 | 1.07 | 1.02 | NA |
| tocopherol | | | | | | | | | | |
| luteolin | 0.96 | 0.98 | 1.00 | 1.00 | 1.01 | 0.84 | 0.37 | 0.13 | 0.10 | 74.20 |
| apigenin | 1.01 | 1.02 | 1.02 | 0.98 | 1.02 | 1.00 | 0.82 | 0.67 | 0.62 | NA |

TABLE 7.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.00 | 1.01 | 1.00 | 1.06 | 1.09 | 1.02 | 1.03 | 1.03 | 1.00 | NA |
| butyrate | 0.95 | 0.96 | 0.99 | 1.00 | 1.02 | 1.01 | 0.90 | 0.67 | 0.27 | 5066.53 |
| beta- | 1.00 | 0.99 | 1.01 | 1.02 | 1.05 | 1.01 | 1.01 | 1.01 | 0.98 | >10000 |
| hydroxybutyrate | | | | | | | | | | |
| butanediol | 0.95 | 0.97 | 1.03 | 1.01 | 1.00 | 1.02 | 1.01 | 0.97 | 0.98 | NA |
| propionate | 0.98 | 0.98 | 1.02 | 1.03 | 1.01 | 1.04 | 1.04 | 0.94 | 0.65 | NA |
| valerate | 0.98 | 1.00 | 1.01 | 0.97 | 1.04 | 1.02 | 0.98 | 0.87 | 0.62 | >10000 |

Assay 6.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in PC-3 prostate cancer cells. Cytotoxicity was assessed at nine concentration levels for each compound and the 50% inhibition concentration (IC50) determined for each compound. The data are shown in Tables 8.1 and 8.2.

TABLE 8.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 1.01 | 1.02 | 1.01 | 1.02 | 1.03 | 0.96 | 0.95 | 0.32 | 0.00 | 248.32 |
| EGCG | 1.01 | 0.99 | 1.01 | 0.97 | 0.99 | 0.82 | 0.60 | 0.51 | 0.10 | 244.78 |
| urolithin C | 1.03 | 1.00 | 1.04 | 1.01 | 1.07 | 0.93 | 0.08 | 0.04 | 0.03 | 52.65 |
| L-arabinose | 1.03 | 1.00 | 1.00 | 1.01 | 1.03 | 1.01 | 1.00 | 1.01 | 1.00 | NA |
| EGC | 1.03 | 0.99 | 1.00 | 1.01 | 1.02 | 0.98 | 0.96 | 0.96 | 0.36 | 899.32 |
| myricetin | 1.03 | 0.99 | 1.03 | 1.00 | 1.05 | 1.01 | 1.02 | 1.01 | 0.88 | NA |
| alpha- | 1.01 | 0.99 | 1.04 | 1.00 | 1.03 | 1.00 | 0.99 | 1.02 | 1.01 | NA |
| tocopherol | | | | | | | | | | |
| luteolin | 1.00 | 0.99 | 1.00 | 1.02 | 0.96 | 0.74 | 0.52 | 0.23 | 0.23 | 93.45 |
| apigenin | 1.00 | 1.00 | 0.98 | 1.02 | 1.07 | 0.98 | 0.82 | 0.72 | 0.70 | NA |

TABLE 8.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.05 | 1.00 | 1.02 | 1.03 | 1.07 | 1.05 | 1.01 | 1.00 | 0.88 | >10000 |
| butyrate | 1.00 | 0.99 | 1.00 | 1.00 | 0.98 | 0.93 | 0.82 | 0.66 | 0.40 | 6444.47 |
| beta- | 1.01 | 1.00 | 1.01 | 1.03 | 1.03 | 1.03 | 1.01 | 1.00 | 0.97 | NA |
| hydroxybutyrate | | | | | | | | | | |
| butanediol | 1.00 | 0.99 | 1.03 | 1.02 | 1.00 | 1.02 | 0.97 | 0.98 | 0.96 | NA |
| propionate | 1.00 | 0.98 | 0.98 | 1.01 | 1.04 | 0.98 | 0.94 | 0.84 | 0.66 | >10000 |
| valerate | 1.00 | 0.99 | 1.02 | 1.01 | 1.04 | 0.97 | 0.94 | 0.84 | 0.69 | >10000 |

Assay 7.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in A-431 skin cancer cells. Cytotoxicity was assessed at nine concentration levels for each compound and the 50% inhibition concentration (IC50) determined for each compound. The data are shown in Tables 9.1 and 9.2.

TABLE 9.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 1.01 | 1.01 | 1.02 | 1.02 | 0.98 | 0.87 | 0.65 | 0.13 | 0.00 | 132.33 |
| EGCG | 1.02 | 1.02 | 1.02 | 1.01 | 1.04 | 0.78 | 0.38 | 0.30 | 0.04 | 81.91 |
| urolithin C | 1.02 | 1.02 | 1.02 | 1.03 | 1.02 | 0.95 | 0.34 | 0.20 | 0.06 | 76.72 |
| L-arabinose | 1.03 | 1.02 | 1.02 | 1.03 | 1.03 | 1.05 | 1.05 | 1.05 | 1.02 | NA |
| EGC | 1.03 | 1.03 | 1.02 | 1.02 | 0.98 | 0.96 | 0.95 | 0.60 | 0.00 | 384.64 |
| myricetin | 1.02 | 1.00 | 1.02 | 1.02 | 1.00 | 0.96 | 0.87 | 0.87 | 0.78 | >1000 |
| alpha- | 1.00 | 0.99 | 1.01 | 1.01 | 1.03 | 1.01 | 1.02 | 1.01 | 1.04 | NA |
| tocopherol | | | | | | | | | | |
| luteolin | 1.01 | 0.99 | 1.02 | 1.01 | 0.99 | 0.61 | 0.39 | 0.20 | 0.04 | 71.83 |
| apigenin | 1.01 | 1.00 | 1.00 | 1.02 | 1.00 | 0.83 | 0.58 | 0.47 | 0.45 | 178.16 |

TABLE 9.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 1.02 | 1.03 | 1.06 | 1.03 | 1.05 | 1.05 | 1.05 | 1.02 | 0.99 | NA |
| butyrate | 1.03 | 1.02 | 1.02 | 1.03 | 0.99 | 0.96 | 0.85 | 0.67 | 0.37 | 6321.85 |
| beta- | 1.01 | 1.02 | 1.03 | 1.03 | 1.03 | 1.05 | 1.05 | 1.03 | 1.01 | NA |
| hydroxybutyrate | | | | | | | | | | |
| butanediol | 1.02 | 1.03 | 1.04 | 1.04 | 1.03 | 1.04 | 1.02 | 1.02 | 1.01 | NA |
| propionate | 0.99 | 1.01 | 1.03 | 1.02 | 1.04 | 0.99 | 0.98 | 0.89 | 0.70 | >10000 |
| valerate | 1.01 | 1.01 | 1.02 | 1.02 | 1.02 | 1.03 | 0.98 | 0.89 | 0.71 | NA |

Assay 8.

Resveratrol, epigallocatechin-gallate, urolithin C, acetate, butyrate, beta-hydroxybutyrate, butanediol, L-arabinose, epigallocatechin, myricetin, propionate, alpha-tocophenol, luteolin, valerate, and apigenin were assayed as described above in AGS stomach cancer cells. Cytotoxicity was assessed at nine concentration levels for each compound and the 50% inhibition concentration (IC50) determined for each compound. The data are shown in Tables 10.1 and 10.2.

TABLE 10.1

| | Cell survival at given concentration | | | | | | | | | |
|---|---|---|---|---|---|---|---|---|---|---|
| | 101 | 318 | 1 | 3 | 10 | 32 | 100 | 316 | 1000 | IC50 |
| | nM | nM | μM | μM | μM | μM | μM | μM | μM | (μM) |
| resveratrol | 1.00 | 0.95 | 1.01 | 0.98 | 1.00 | 1.01 | 0.94 | 0.33 | 0.00 | 251.56 |
| EGCG | 1.01 | 1.02 | 1.05 | 1.02 | 0.90 | 0.49 | 0.20 | 0.08 | 0.00 | 33.21 |
| urolithin C | 0.98 | 0.99 | 0.98 | 0.99 | 1.02 | 0.86 | 0.15 | 0.04 | 0.02 | 55.97 |
| L-arabinose | 1.00 | 1.02 | 1.01 | 1.02 | 1.02 | 1.04 | 1.05 | 1.02 | 1.01 | NA |
| EGC | 1.03 | 1.03 | 1.08 | 1.05 | 1.04 | 1.04 | 1.07 | 0.80 | 0.08 | 476.83 |
| myricetin | 1.07 | 1.03 | 1.10 | 1.03 | 1.09 | 1.11 | 1.08 | 1.07 | 0.81 | NA |
| alpha- | 1.03 | 1.03 | 1.07 | 1.04 | 1.07 | 1.06 | 1.09 | 1.06 | 1.06 | NA |
| tocopherol | | | | | | | | | | |
| luteolin | 0.99 | 1.00 | 1.03 | 1.06 | 1.06 | 0.77 | 0.29 | 0.08 | 0.03 | 68.15 |
| apigenin | 0.99 | 1.02 | 1.04 | 1.01 | 1.06 | 1.02 | 0.89 | 0.71 | 0.64 | NA |

TABLE 10.2

| | Cell survival at given concentration | | | | | | | | | |
|---|---|---|---|---|---|---|---|---|---|---|
| | 1 | 3 | 10 | 32 | 100 | 317 | 1 | 3 | 10 | IC50 |
| | μM | μM | μM | μM | μM | μM | mM | mM | mM | (μM) |
| acetate | 0.99 | 1.00 | 1.02 | 1.03 | 1.03 | 1.03 | 1.02 | 1.03 | 1.02 | NA |
| butyrate | 1.00 | 1.00 | 1.02 | 1.01 | 1.04 | 1.05 | 0.91 | 0.65 | 0.11 | 4459.34 |
| beta- | 1.01 | 1.03 | 1.01 | 1.02 | 1.04 | 1.03 | 1.04 | 1.03 | 1.00 | NA |
| hydroxybutyrate | | | | | | | | | | |
| butanediol | 1.01 | 1.02 | 1.03 | 1.05 | 1.04 | 1.02 | 1.06 | 1.02 | 0.98 | NA |
| propionate | 1.02 | 0.98 | 1.03 | 1.01 | 1.04 | 1.01 | 1.03 | 0.96 | 0.70 | NA |
| valerate | 1.03 | 1.03 | 1.04 | 1.03 | 1.05 | 1.06 | 1.06 | 0.97 | 0.63 | NA |

### Example 5. Small Molecules Inhibit Tumor Growth in Nude Mice

All in vivo studies are performed in accordance with institutional, state and federal guidelines. Female BALB-nu/nu mice (CAnN.Cg-Foxn1nu/CrlCrlj nu/nu) are obtained from Charles River Laboratories or another source and maintained under pathogen-free conditions. These mice are given access to standard mouse chow and water ad libitum. HCT116 human colon carcinoma cells (ATCC) are maintained under serum-free conditions using McCoy's 5A medium supplemented with 4 ug/mL of transferrin, 5 ug/mL of insulin, and 10 ng/mL of EGF. 5×10⁶ HCT116 tumor cells/mouse are injected subcutaneously into the right flank of the 7-9-week-old mice. When tumor volume reaches 200 mm³ (day 0), the mice are randomized, and treated with vehicle or compounds from Example 1 to be administered using the preferred route of administration or vehicle. Route of administration may include incorporation into chow, oral gavage, intraperitoneal, intravenous, intramuscular, rectal, or intravaginal administration.

Preferred vehicles for drug delivery are well known and specific to the route of administration; for example, 2-hydroxypropyl-β-cyclodextrin (HPCD) solution in distilled water for oral delivery. The compounds may be administered at doses ranging from 0.01 mg/kg to 1000 mg/kg administered and delivered once a day (qd), twice a day (bid), or three times a day (tid). The dose will not exceed the maximum tolerated dose (MTD). The MTD is defined as the highest dose that produced less than 10% weight loss and no mortality. Tumor size is measured 3 times/weeks using calipers and the volume calculated: V=(W(2)×L)/2 where Vis tumor volume, W is tumor width, Lis tumor length. Tumor growth inhibition (TGI) is calculated using the following formula: % TGI is defined as (1−(mean volume of treated tumors)/(mean volume of control tumors))×100%.

### Example 6. Small Molecules Inhibit Tumor Growth in an Orthotopic Model of Colon Cancer in Mice

Construction of GFP Expressing Human Colon Tumor Cells

HCT116 cells (ATCC) are maintained under serum-free conditions using McCoy's 5A medium supplemented with 4 ug/mL of transferrin, 5 ug/mL of insulin, and 10 ng/mL of EGF. HCT116 Green Fluorescence Protein (GFP) Transfection Packaging cells, 293 GP (Clontech, Mountain View, Calif.), are co-transfected with a plasmid encoding VSVG envelope protein and a retroviral vector encoding GFP and the G418 resistance gene using FuGene (Invitrogen, Carlsbad, Calif.). The viruses are collected 48 h later and used to infect HCT116 cells. After 48 h, the infected HCT 116 cells were selected by treatment with G418 for 5 d. This resulted in a stable transfection.

Orthotopic Implantation and Imaging

All in vivo studies are performed in accordance with institutional, state and federal guidelines. BALB-nu/nu mice are obtained from Charles River Laboratories or another source and maintained under pathogen-free conditions. These mice are given access to standard mouse chow and water ad libitum. Five×106 HCT116 GFP labeled cells are subcutaneously injected into BALB/c nude male mice. At 1 cm³, the xenograft is excised and minced for implantation into other 4 to 6-week-old male BALB/c nude mice. The recipient animals are anesthetized with isoflurane inhalation and a 1 cm laparotomy is performed. Two 1 mm³ pieces are subserosally implanted on to the ceca and ascending colons of 32 other BALB/c nude male mice. When tumor volume reached 200 mm³ (day 0), the mice are randomized, and treated with vehicle or test compounds from Example 1 using the preferred route of administration. Preferred route of administration included incorporation into chow, oral gavage (p.o.), intraperitoneal (i.p.), intravenous (i.v.), subcutaneous (s.c.) and intramuscular (i.m.). Preferred vehicles for compound delivery are well known and specific to the route of administration; for example, 2-hydroxypropyl-β-cyclodextrin (HPCD) solution in distilled water for oral delivery. Compounds may be administered at doses ranging from 0.01 mg/kg to 1000 mg/kg administered and delivered once a day (qd), twice a day (bid), or three times a day (tid). Subsequently, animals are anesthetized with a 1:1 mixture of ketamine (10 mg/mL) and xylazine (1 mg/mL) with intraperitoneal injection (0.01 mL/mg) and weekly GFP fluorescence imaging is performed for up to 8 week at which time all animals are to be euthanized and necropsied. Each week, after measuring body weight, the orthotopic tumor size in each individual animal is measured using an IFLUOR-100 small animal in vivo fluorescence imaging system, and tumor volume is calculated using Image-Pro software (Media Cybernetics, Silver Spring, Md., USA) based on the length (L) and width (W) of the tumor. In addition, a representative 1392×1040 resolution picture of each of the tumors is taken under the fluorescence imaging system at the same time. Real-time determination of tumor burden is assessed by estimating fluorescent surface area of tumor as a function of time and treatment. Excised tissues are fixed in 10% buffered formalin and embedded in paraffin. Slides are cut and stained with hematoxylin and eosin (H&E) to evaluate local invasion and distant colony formation. Random single sections through the liver and lung parenchyma are taken to evaluate for metastases.

### Example 7: Human Regulatory T Cell Differentiation Assay

Peripheral blood mononuclear cells (PBMCs) from whole blood donated by health volunteers were separated by Ficoll-Paque gradient centrifugation and naïve CD4⁺ T cells were subsequently isolated using magnet beads (EasySep™ Human Naïve CD4⁺ T Cell Isolation Kit, Cambridge, Mass.). For regulatory T cell (Treg) differentiation assay, naïve CD4⁺ T cells were cultured (1-10×10⁴ cells) in CTS OpTmizer medium for 6 days and stimulated with 5 ng/ml TGF-β, 100 U/ml IL-2, and ImmunoCult™ Human CC3/CD28/CD2 T Cell Activator; Stemcell #10990) with/without Compounds. Cell viability was determined using a viability dye (eBioscience Fixable Viability Dye eFluor 780: ThermoFisher 65-0865-14) at 1:500 dilution. The cells were gated for Treg, defined as Live, CD11c⁻, CD14⁻, CD19⁻, CD8⁻, CD4⁺, CD3⁺, CD25⁺, FOXP3⁺. Percent (%) Tregs were calculated as percentage of CD4⁺, CD25⁺, FOXP3⁺ cells over total CD4⁺ T cells. Statistical analysis was performed with GraphPad Prism Software Using One-Way ANOVA.

TABLE 11

| | Treg induction | Cell viability |
|---|---|---|
| Treatment | % DMSO | % DMSO |
| Acetic acid 1 mM | + | = |
| Acetic acid 3 mM | ++ | = |
| L-Arabinose 0.5 mM | = | = |
| L-Arabinose 1 mM | = | = |
| EGCG 100 nM | = | = |
| EGCG 1 uM | = | = |
| Quercetin 100 nM | = | = |
| Quercetin 1 uM | = | = |
| (R)-1,3-Butanediol 100 uM | = | = |
| (R)-1,3-Butanediol 0.5 mM | = | = |
| Sodium BHB 2 mM | + | = |
| Sodium BHB 20 mM | = | − |
| Butyric Acid 3 mM | − | − |
| Propionic acid 3 mM | ++ | = |
| Rosiglitazone 10 uM | = | = |
| Rosiglitazone 100 uM | = | − |
| Resveratrol 1 uM | + | − |
| Resveratrol 10 uM | + | − |
| Obeticholic acid 100 uM | + | = |
| DMSO | =(100.0) | =(100%) |

<90%: −
90% ≥ ≤110%: =
110% > ≤130%: +
130%>: ++

Table 11 shows compounds that increased the differentiation of naïve CD4⁺ T cells into Tregs (+, ++), or decreased the differentiation of naïve CD4⁺ T cells into Tregs (−). Tregs play an important role in keeping the balance of immune system and compounds that increase Tregs (+, ++) may be useful in the treatment of autoimmune and inflammatory diseases, whereas compounds that reduce Tregs (−) may enhance the efficacy of immunotherapy in cancer patients. Examples of cancers include non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, colorectal cancer, small intestine cancer, acute lymphoblastic leukemia, acute myeloid leukemia, adrenocortical carcinoma, Kaposi sarcoma, primary CNS lymphoma, anal cancer, astrocytoma, glioblastoma, bladder cancer, Ewing sarcoma, osteosarcoma, non-Hodgkin lymphoma, breast cancer, brain tumor, cervical cancer, bile duct cancer, chronic lymphocytic leukemia, chronic myelogenous leukemia, gallbladder cancer, gastrointestinal stromal tumor, ovarian cancer, testicular cancer, multiple myeloma, neuroblastoma, pancreatic cancer, parathyroid cancer, prostate cancer, rectal cancer, and Wilms tumor.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of modulating a cancer marker in a subject in need thereof, the method comprising administering to the subject an effective amount of an acylated active agent selected from the group consisting of an acylated catechin polyphenol, acylated stilbenoid, acylated ellagic acid, acylated ellagic acid analogue, and acylated ketone body or pre-ketone body.

2. The method of claim 1, wherein the cancer marker is for a cancer selected from the group consisting of stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, and colorectal cancer.

3. The method of claim 2, wherein the cancer marker is for a cancer selected from the group consisting of stomach cancer, skin cancer, prostate cancer, lung cancer, breast cancer, non-Hodgkin lymphoma, and bladder cancer.

4. A method of treating a cancer in a subject in need thereof, the method comprising administering to the subject an effective amount of an acylated active agent selected from the group consisting of an acylated catechin polyphenol, acylated stilbenoid, acylated ellagic acid, acylated ellagic acid analogue, and acylated ketone body or pre-ketone body.

5. The method of claim 4, wherein the cancer is stomach cancer, skin cancer, prostate cancer, lung cancer, breast cancer, non-Hodgkin lymphoma, bladder cancer, non-small cell lung cancer, squamous cell carcinoma of the head and neck, classical Hodgkin's lymphoma, urothelial carcinoma, melanoma, renal cell carcinoma, hepatocellular carcinoma, Merkel cell carcinoma, carcinomas with microsatellite instability, or colorectal cancer.

6. The method of claim 5, wherein the cancer is stomach cancer, skin cancer, prostate cancer, lung cancer, breast cancer, non-Hodgkin lymphoma, bladder cancer, or colorectal cancer.

7. The method of any one of claims 1 to 6, wherein a CD4⁺CD25⁺ Treg cell count, cytotoxic T cell count, interferon γ (IFNγ) level, interleukin-17 (IL17) level, or intercellular adhesion molecule (ICAM) level is increased following the administration of the acylated active agent.

8. The method of any one of claims 1 to 7, wherein an NFκB level, matrix metallopeptidase 9 (MMP9) level, 8-iso-prostaglandin F_{2α} (8-iso-PGF2α) level, or CXCL13 level is reduced following the administration of the acylated active agent.

9. The method of any one of claims 1 to 8, wherein a Tₕ1 cell count, IgA level, or inducible nitric oxide synthase (iNOS) level is modulated following the administration of the acylated active agent.

10. The method of any one of claims 1 to 9, wherein the method comprises administering the acylated active agent to the subject orally.

11. The method of claim 10, wherein, following oral administration to the subject, the acylated active agent is hydrolyzable in the gastrointestinal tract of the subject.

12. The method of any one of claims 1 to 11, wherein the acylated active agent is hydrolyzed to release at least one fatty acid acyl as a fatty acid.

13. The method of claim 12, wherein the fatty acid is a C₃₋₅ fatty acid.

14. The method of claim 13, wherein the fatty acid is propionic acid, butyric acid, or valeric acid.

15. The method of claim 14, wherein the fatty acid is butyric acid.

16. The method of any one of claims 1 to 15, wherein the acylated active agent is acylated catechin polyphenol.

17. The method of claim 16, wherein the acylated catechin polyphenol is a compound of formula (I): 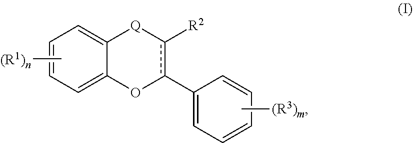
or a pharmaceutically acceptable salt thereof,
wherein [symbol]
is a single carbon-carbon bond or double carbon-carbon bond;
Q is —CH₂— or —C(O)—;
each R¹ and each R³ is independently H, halogen, —OR^{A}, phosphate, or sulfate;
R² is H or —OR^{A};
each R^{A} is independently H, optionally substituted alkyl, a monosaccharide, a sugar acid, a group containing a fatty acid, or benzoyl optionally substituted with 1, 2, 3, or 4 substituents independently selected from the group consisting of H, hydroxy, halogen, a group containing a fatty acid, an optionally substituted alkyl, an optionally substituted alkoxy, a monosaccharide, a sugar acid, phosphate, and sulfate;
each of n and m is independently 0, 1, 2, 3, or 4;
provided that the compound of formula (I) comprises at least one group containing a fatty acid.

18. The method of claim 17, wherein at least one R¹ is —OR^{A}, in which R^{A} is a group containing a fatty acid.

19. The method of claim 17 or 18, wherein the acylated catechin polyphenol is a compound of formula (I-a):

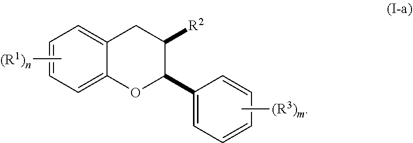

20. The method of claim 17 or 18, wherein the acylated catechin polyphenol is a compound of formula (I-b):

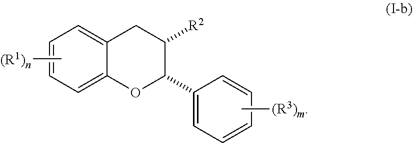

21. The method of claim 17 or 18, wherein the acylated catechin polyphenol is a compound of formula (I-c):

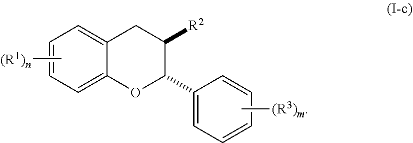

22. The method of claim 17 or 18, wherein the acylated catechin polyphenol is a compound of formula (I-d):

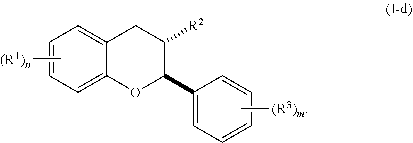

23. The method of claim 17 or 18, wherein the acylated catechin polyphenol is a compound of formula (I-f):

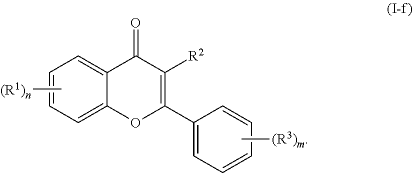

24. The method of any one of claims 17 to 23, wherein n is 2.

25. The method of any one of claims 17 to 24, wherein m is 1.

26. The method of any one of claims 17 to 24, wherein m is 2.

27. The method of any one of claims 17 to 24, wherein m is 3.

28. The method of any one of claims 17 to 27, wherein each R¹ is independently —OR^{A}.

29. The method of any one of claims 17 to 28, wherein each R³ is independently —OR^{A}.

30. The method of any one of claims 17 to 29, wherein R² is H or —OR^{A}.

31. The method of any one of claims 17 to 30, wherein each R^{A} is independently H, optionally substituted alkyl, or a group containing a fatty acid.

32. The method of claim 17, wherein the acylated catechin polyphenol is a compound is of formula (I-e): 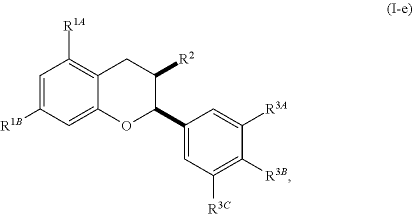
or a pharmaceutically acceptable salt thereof,
wherein each of R^{1A} and R^{1B} is independently as defined for R¹; and each of R^{3A}, R^{3B}, and R^{3C} is independently as defined for R³.

33. The method of claim 32, wherein each of R^{1A} and R^{1B} is independently —OR^{A}.

34. The method of claim 32 or 33, wherein each of R^{3A}, R^{3B}, and R^{3C} is independently H, halogen, or —OR^{A}.

35. The method of any one of claims 17 to 34, wherein R² is a group of formula: 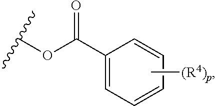
wherein p is 1, 2, 3, or 4, and each R⁴ is independently selected from the group consisting of H, hydroxy, halogen, a group containing a fatty acid, an optionally substituted alkyl, an optionally substituted alkoxy, a monosaccharide, a sugar acid, phosphate, and sulfate.

36. The method of claim 35, wherein p is 3.

37. The method of claim 35 or 36, wherein each R⁴ is independently H, hydroxy, halogen, a group containing a fatty acid, or an optionally substituted alkoxy.

38. The method of any one of claims 35 to 37, wherein R² is a group of formula: 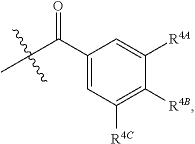
and
each of R^{4A}, R^{4B}, and R^{4C} is as defined for R⁴.

39. The method of claim 38, wherein each of R^{4A}, R^{4B}, and R^{4C} is independently H, hydroxy, halogen, a group containing a fatty acid, or an optionally substituted alkoxy.

40. The method of any one of claims 17 to 39, wherein each R^{A} is independently H, optionally substituted alkyl, fatty acid acyl, or optionally acylated monosaccharide.

41. The method of any one of claims 1 to 15, wherein the acylated active agent is an acylated stilbenoid.

42. The method of claim 41, wherein the acylated stilbenoid is resveratrol having at least one hydroxyl substituted with a group containing a fatty acid.

43. The method of claim 41, wherein the acylated stilbenoid is a compound of the following structure: 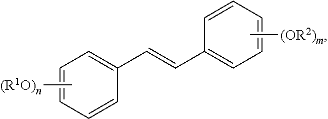
wherein
n is 1, 2, 3, or 4;
m is 1, 2, 3, or 4;
each R¹ is independently H, alkyl, acyl, or a group containing a fatty acid; and
each R² is independently H, alkyl, acyl, or a group containing a fatty acid;
wherein at least one group containing a fatty acid, when present, is independently a monosaccharide having one, two, three, or four hydroxyls substituted with fatty acid acyls;
provided that at least one R¹ or at least one R² is a group containing a fatty acid, or a group containing a ketone body or pre-ketone body.

44. The method of claim 43, wherein n is 1.

45. The method of claim 43 or 44, wherein m is 2.

46. The method of claim 43, wherein the acylated stilbenoid is a compound of the following structure:

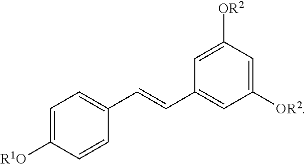

47. The method of any one of claims 43 to 46, wherein at least one R¹ is a group containing a fatty acid.

48. The method of any one of claims 43 to 47, wherein at least one R² is a group containing a fatty acid.

49. The method of any one of claims 1 to 15, wherein the acylated active agent is an acylated ellagic acid.

50. The method of any one of claims 1 to 15, wherein the acylated active agent is an acylated ellagic acid analogue.

51. The method of claim 50, wherein the acylated active agent analogue is urolithin C having at least one hydroxyl substituted with a group containing a fatty acid or a group containing a ketone body or pre-ketone body.

52. The method of any one of claims 1 to 15, wherein the acylated active agent is an acylated ketone body or pre-ketone body.

53. The method of claim 52, wherein the acylated active agent has a ketone body core.

54. The method of claim 52, wherein the acylated active agent has a pre-ketone body core.

55. The method of any one of claims 52 to 54, wherein the acylated ketone body or pre-ketone body comprises at least one group containing a fatty acid.

56. The method of any one of claims 1 to 55, wherein the acylated active agent comprises at least one fatty acid acyl.

57. The method of claim 56, wherein the fatty acid acyl is a short chain fatty acid acyl.

58. The method of claim 57, wherein the short chain fatty acid acyl is acetyl, propionyl, or butyryl.

59. The method of claim 58, wherein the short chain fatty acid acyl is acetyl.

60. The method of any one of claims 1 to 59, wherein the subject is human.

61. A pharmaceutical composition comprising an acylated catechin polyphenol, acylated stilbenoid, acylated ellagic acid, acylated ellagic acid analogue, or acylated ketone body or pre-ketone body,
provided that
the acylated catechin polyphenol is not a fatty acid peracylated epigallocatechin gallate, fatty acid peracylated gallocatechin gallate, fatty acid peracylated epicatechin gallate, or fatty acid peracylated catechin gallate; and
when the core of the acylated catechin polyphenol is selected from the group consisting of epigallocatechin, epigallocatechin gallate, gallocatechin, gallocatechin gallate, catechin, and catechin gallate, at least one hydroxyl attached to the chromane core is substituted.

62. The pharmaceutical composition of claim 61, wherein the pharmaceutical composition comprises an acylated catechin polyphenol.

63. The pharmaceutical composition of claim 61 or 62, wherein acylated catechin polyphenol comprises myricetin or quercetin core.

64. The pharmaceutical composition of claim 61, wherein the pharmaceutical composition comprises an acylated stilbenoid.

65. The pharmaceutical composition of claim 61 or 64, wherein the acylated stilbenoid comprises a resveratrol or piceattanol core.

66. The pharmaceutical composition of claim 61, wherein the pharmaceutical composition comprises an acylated ellagic acid.

66. The pharmaceutical composition of claim 61, wherein the pharmaceutical composition comprises an acylated ellagic acid analogue.

67. The pharmaceutical composition of claim 66, wherein the pharmaceutical composition comprises an acylated ketone body or pre-ketone body.
